(19) United States
(12) Patent Application Publication
(10) Pub. No.: US 2024/0042053 A1
(43) Pub. Date: Feb. 8, 2024

(54) GLYCOCONJUGATES

(71) Applicant: UNIVERSITY OF GEORGIA RESEARCH FOUNDATION, INC., Athens, GA (US)

(72) Inventors: Gerardus Josephus BOONS, Athens, GA (US); Xiuru LI, Athens, GA (US); Patricius Hendrikus Cornelis VAN BERKEL, Epalinges (CH)

(21) Appl. No.: 18/248,331

(22) PCT Filed: Oct. 14, 2021
(86) PCT No.: PCT/US2021/055064

§ 371 Date: Apr. 7, 2023

Related U.S. Application Data
(60) Provisional application No. 63/092,640, filed on Oct. 16, 2020.

(51) Int. Cl. A61K 47/68

(52) U.S. Cl. CPC A61K 47/6889; A61K 47/6805; A61K 47/68033

(57) ABSTRACT

Disclosed herein are glycoconjugates having a cell-binding agent, such as an antibody, conjugating to a payload, such as a drug. The drug is conjugated to the cell-binding agent through an oligosaccharide linker. The glycoconjugates conjugated through the oligosaccharide linkers disclosed herein exhibit improved properties over prior glycoconjugates.

### EARLIER APPLICATION

This application claims priority from United States provisional application number U.S. 63/092,640, filed 16 Oct. 2020. The priority application is hereby incorporated by reference in its entirety and for any and all purposes as if fully set forth herein.

### FIELD OF THE INVENTION

The invention is directed to glycoconjugates having a cell-binding agent, such as an antibody, conjugating to a payload, such as a drug. The drug is conjugated to the cell-binding agent through an oligosaccharide linker.

### BACKGROUND

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

The field has been advanced by the emergence of new classes of potent toxins, such as taxanes, calicheamycins, maytansins, duocarmycins, and auristatins. The low nanomolar to picomolar toxicity of these substances has provided significant advantages over earlier generations of toxins. Another technological advance involves the use of optimized linkers that are hydrolysable in the cytoplasm, resistant or susceptible to proteases, or resistant to multi-drug resistance efflux pumps that are associated with highly cytotoxic drugs.

A common mode for preparing ADCs is the conjugation of the payload (eg. a drug-linker molecule) to the side chain of antibody amino acid lysine or cysteine. The kinetics of lysine addition means conjugation at this residue takes place preferentially at lysine side chains with high steric accessibility and low pKa, making the site-specificity of the reaction difficult to control. More site specificity is offered by conjugating to cysteines, since there are typically no free cysteine sulfhydryl groups present in a native-type antibody under normal conditions. This allows for methods where free sulfhydryl groups can be introduced into the antibody molecule by, for example, selective reduction of existing cysteine or the introduction of additional cysteines through protein engineering. In both case, payloads can be effectively conjugated to the free sulfhydryl groups using, for example, electrophilic alkylation based on maleimide addition. This method allows for efficient and site-selective generation of conjugates. However, given the benefits of high product homogeneity and conjugates with high resistance to off-target release of payload, research has continued to identify conjugation methods offering further improvements over sulfhydryl alkylation.

One alternative conjugation technology makes use of azide chemistry (N3 groups, also referred to as azido groups). In particular, azide groups are able to undergo selective cycloaddition with terminal alkynes (copper-catalyzed) or with cyclic alkynes (copper free, with the reaction promoted by ring strain). The triazoles resulting from reaction with alkynes are particularly resistant to hydrolysis and other degradation pathways. This reaction has been shown to have utility in the production of ADCs (see, for example, WO2014/065661, and Li et al., Angew. Chem. Int. Ed. Engl., 2014, Jul. 7; 53(28):7179-82). Potential use in ADC production has also been discussed for ketones plus either hydroxylamines or hydrazines (see WO2014/065661).

A number of strategies exist for introducing the above functional groups into conjugate precursors have been discussed. One strategy that has been demonstrated to yield safe and effective ADCs involves conjugation of the payload to the glycan moiety of a glycosylated cell-binding agent, such as an antibody (see, for example, WO/2018/146189).

Conjugation via glycans is a potentially versatile strategy for ADC generation, as—for example—all IgG antibodies expressed in mammalian or yeast cell cultures bear a N-linked glycan moiety on the Fc portion of each heavy chain. However, this methodology presents a number of challenges. For example, glycans are typically present as a complex mixture of isoforms, which may contain different levels of galactosylation (G0, G1, G2) and fucosylation (G0F, G1F, G2F) which may in turn lead to undesirable heterogeneity in conjugation stoichiometry. Accordingly, existing methods often employ one or more ‘glycan remodelling’ steps in which enzymes are used to trim and/or add carbohydrate moieties in order to homogenise the glycan structures as much as possible prior to conjugation with the payload (see WO 2007/133855, WO2014/065661, and Li et al., Angew Chem Int Ed Engl., 2014, Jul. 7; 53(28):7179-82). However, the huge variety of possible sugar moieties, linkages, branching, chain length, and modifying enzymes available mean there is a vast genus of possible final structures of the glycan moiety. The final size and structure of the glycan influences many key properties of the final glycoconjugate (e.g. drug-antibody-ratio, conjugate hydrophilicity, conjugate hydrodynamic etc.) many of which cannot be reliably predicted a priori. Accordingly, research into advantageous glycan configurations is ongoing.

Once the glycoprotein has been remodeled there are several possible strategies for conjugation to a payload. For example, numerous methods have been described involving the condensation of the homogenized glycoprotein with singly or multiply azide or alkyne-functionalized saccharides to yield activated glycoprotein intermediates which are then conjugated to payloads using the above-described chemistry (for additional detail see discussion in, for example, WO2014/06566, Li et al., Angew Chem Int Ed Engl., 2014, Jul. 7; 53(28):7179-82, and the references cited therein).

The above methods have been demonstrated to yield glycoconjugates having in vivo anti-cancer efficacy (see, for example, WO/2018/146189). Nonetheless, research to further improve the properties of such glycoconjugates across a range of cell-binding agents and payloads is ongoing.

### SUMMARY

The present inventors have investigated the properties of a range of oligosaccharide structures were investigated with the aim of identifying oligosaccharide structures that both (1) allowed for advantageous glycoconjugate properties, and (2) were readily amenable to commercial-scale manufacture.

During their investigation, the inventors discovered that glycoconjugates having a relatively short trisaccharide moiety of -GlcNAc-Gal-Sia- between the cell binding agent and payload had a range of advantageous properties. For example, compared to otherwise similar glycoconjugates having larger oligosaccharide linkers this class of glycoconjugates was unexpectedly found to: have higher hydrophilicity and solubility; significantly faster kinetics of conjugation; significantly more efficacious in vivo (despite similar in vitro activity); better control/consistency of achieving drug-antibody ratio (“DAR”)=2; and significantly improved tolerability of treatment by subjects. Without wishing to be bound by theory, the present inventors believe that these properties arise in part due to the presence and location of the negatively charged sialic residue. For some payloads this was found to be associated with improved glycoconjugate efficacy as compared to uncharged sugar moieties in the same position.

The present inventors further determined that the advantageous -GlcNAc-Gal-Sia-glycoconjugates could be manufactured using readily-available enzyme catalysts. In particular, it was unexpectedly found that certain galactosyl transferases were able to efficiently transfer galactose onto a GlcNAc residue, preferably α-linked to an Asn residue in the peptide backbone, (optionally bearing α1-6 fucose), despite that reaction not occurring in the natural system in which this enzyme is found. The galactosylated oligosaccharide resulting from that reaction was also readily susceptible to the addition of a modified sialic acid by ST6Gal1 sialyltransferase. The modified sialic acid residue can then be efficiently coupled to a wide range of payloads, for instance cytotoxic drugs and other therapeutic agents.

The details of one or more embodiments are set forth in the descriptions below. Other features, objects, and advantages will be apparent from the description and from the claims.

### BRIEF DESCRIPTION OF THE FIGURES

FIG. 1 depicts a glycosylation remodeling and conjugation according to Approach 1. GlcNAc=A-acetyl-glucosamine, Man=mannose, Gal=galactose, Fuc=fucose, Sia=sialic acid, WH=warhead molecule. Reaction conditions: (i) UDP-Galactose, galactosyltransferase, MOPS buffer (50 mM, 20 mM MnCl2, pH 7.2), 24 h; (ii) CMP-Neu5N3, ST6Gal1 sialyltransferase, 1% BSA, alkaline phosphatase, cacodylate buffer (50 mM, pH 7.6), 24 h; (iii) WH-DIBO.

FIG. 2 depicts a glycosylation remodeling and conjugation according to Approach 2. GlcNAc=A-acetyl-glucosamine, Man=mannose, Gal=galactose, Fuc=fucose, Sia=sialic acid, WH=warhead molecule. Reaction conditions: (i-1) EndoS; (i-2) EndoS/BtFucH; (ii) UDP-Galactose, β4GalT1, MOPS buffer (50 mM, 20 mM MnCl2, pH 7.2), 1% BSA, 1.3% alkaline phosphatase; (iii) CMP-Neu5N3, cacodylate buffer (50 mM, pH 7.6), 1% BSA, 1.3% alkaline phosphatase; (iv) WH-DIBO, CuSO4, Na ascorbate.

FIG. 3 depicts an HIC profile of Her-WH-App1 and Her-WH-App2.

FIG. 4 depicts the in vivo efficacy of Her-WH-App1 and Her-WH-App2 versus the benchmark Her2xADC.

FIG. 5 depicts the pharmacokinetics (PK) of Her-WH-App1 and Her-WH-App2 in rats.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

As used herein, a “clickable group” refers to a functional group that can undergo a cycloaddition reaction with another clickable group under mild conditions that do not denature proteins or other biomacromolecules.

As used herein, a “drug-antibody ratio” or “DAR” refers to the number of drugs, or more generally payloads, conjugated to an individual antibody, or more generally cell-binding agent. A DAR of 1 indicates there is one drug conjugated to an antibody, a DAR of 2 indicates that there are two drugs conjugated to an antibody, etc. The skilled person understands that certain bioconjugation techniques do not install a uniform number of drugs on each antibody in a given sample. Such cases can be defined by a non-integer DAR, e.g., 1.5, indicating that for a given sample, on average there are 1.5 drugs conjugated to each antibody.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cirmolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

The term “nucleotide” refers to a molecule that is composed of a nucleobase, a five-carbon sugar (either ribose or 2-deoxyribose), and one, two or three phosphate groups.

Without the phosphate group, the nucleobase and sugar compose a nucleoside. A nucleotide can thus also be called a nucleoside monophosphate, a nucleoside diphosphate or a nucleoside triphosphate. The nucleobase may be adenine, guanine, cytosine, uracil or thymine. Examples of a nucleotide include uridine diphosphate (UDP), guanosine diphosphate (GDP), thymidine diphosphate (TDP), cytidine diphosphate (CDP) and cytidine monophosphate (CMP).

As used herein, two atoms connected via the symbol [symbol] may be connected via a single or double bond.

As used herein, the term C₍ₓ₎alkylene, wherein x is a number, refers to an unsubstituted carbon chain spacer having the —(CH₂)ₓ—; the term arylene refers to an aromatic ring spacer and heterocyclene refers to a heterocyclic spacer. The substitution pattern may be further specified, e.g., phenylene, naphthalene, imidazoylene, etc. The regiochemistry of the spacer may further be specified, e.g., ortho-phenylene, o-phenyl, and 1,2-phenylene all describe a phenyl ring spacer in which the other groups are bonded to adjacent carbons. Bonding arrangements of heterocyclene spacers may be designated using IUPAC rules for ring numbering. By way of example, X-[1,4-phenylene]-Y refers to a compound having the formula:

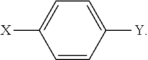

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. In a specific example, groups that are said to be substituted are substituted with a protic group, which is a group that can be protonated or deprotonated, depending on the pH.

It will be appreciated that certain compounds according to the invention may contain one or more centers of asymmetry and may therefore be prepared and isolated as a mixture of isomers such as a racemic or diastereomeric mixture, or in an enantiomerically or diastereomerically pure form. In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined. However, the depiction of a compound without specifying the absolute configuration of an asymmetric center should not be taken as requiring all possible isomers are necessarily present in every embodiment.

Certain compounds of the invention will include ionizable functional groups, including carboxylic acids, sulfonic acids, phosphonic acids, amines, and the like. The skilled person will understand that such groups will contain, or will not contain, an ionizable hydrogen atom depending on pH. Depiction of a particular compound in one state of ionization (e.g., protonated) does not exclude other states (e.g., deprotonated) that would exist at different pH.

Unless specified otherwise, the term “patient” refers to any mammalian animal, including but not limited to, humans.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Disclosed herein are glycoconjugates having at least one payload conjugated to a cell-binding agent (“CBA”). The average number of payloads per CBA in preparations from conjugation reactions may be characterized by conventional means such as UV, reverse phase HPLC, HIC, mass spectroscopy, ELISA assay, and electrophoresis. The quantitative distribution of CBA in terms of p may also be determined. By ELISA, the averaged value of p in a particular preparation of CBA may be determined (Hamblett et al (2004) Clin. Cancer Res. 10:7063-7070; Sanderson et al (2005) Clin. Cancer Res. 11:843-852.

The glycoconjugate compositions described herein can be homogenous, i.e., meaning that each cell binding agent is conjugated to the same number of payloads. In other embodiments, the glycoconjugate compositions can include a distribution of conjugates, i.e., some cell binding agents conjugated to a single payload, some cell binding agents conjugated to two payloads, some cell binding agents conjugated to three payloads, etc. Unless specified to the contrary, the depiction of a cell binding agent conjugated to a certain number of payloads does not exclude the possibility that other conjugates are also present.

In some embodiments, the glycoconjugates disclosed herein have the structure:

[[Payload]ₓ-sialoside-Gal-GlcNAc]_{y}-CBA,

wherein
sialoside refers to a modified sialic acid residue
Gal refers to a galactosyl residue;
GlcNAc refers to a N-acetyl glucosamine residue, optionally glycosylated with fucose at C-6;
Y is an integer selected from 1, 2, 3, 4, 5, 6, 7, or 8;
payload represents one or more therapeutic agents covalently linked to a modified sialic acid residue;
x represent an integer selected from 1-100, 50-100, 25-50, 1-50, 1-25, 1-16, 1-12, 1-8, or 1-4
and CBA refers to a cell binding agent.

In certain embodiments, x can be 1, 2, 3, 4, 5, 6, 7, 8, 9, or 10.

Modified sialic acid residues (or sialosides) have the general formula:

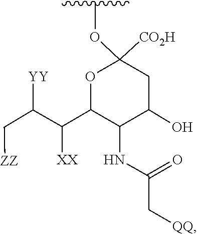

wherein QQ is hydrogen or a conjugated payload;
ZZ is hydroxyl or a conjugated payload;
YY is hydroxyl or a conjugated payload;
XX is hydroxyl or a conjugated payload; and
wherein at least one of QQ, ZZ, YY, and XX is a conjugated payload.

In some embodiments, QQ is a conjugated payload, and each of XX, YY, and ZZ are hydroxyl. In other embodiments ZZ is a conjugated payload, QQ is hydrogen, and XX and YY are each hydroxyl. In yet further embodiments, ZZ and QQ are each conjugated payloads, and XX and YY are each hydroxyl. In such cases ZZ and QQ may be the same or different.

In one embodiment, the average number of payloads per CBA is in the range 1 to 4. In some embodiments the range is selected from 1 to 2, 1 to 3, 2 to 4, 3-6 or 4-8.

In certain aspects of the invention, the glycoconjugates can have the formula:

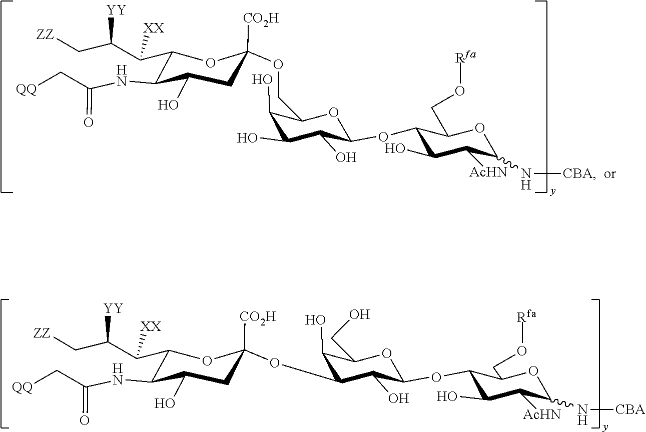

wherein:
CBA is a cell-binding agent, e.g., a peptide such as an antibody, preferably a monoclonal antibody;
y, which is a measure of the DAR, can be from 0.5-6; preferably 0.8-4, more preferably 0.8-2.2, and even more preferably 0.9-2.1. In some embodiments, y is from 0.8-1.2, 0.9-1.1, 1.8-2.2, or 1.9-2.1.

R^{fa} is a hydrogen or fucose moiety;
QQ is hydrogen, or at least one conjugated payload;
ZZ is hydroxyl, or at least one conjugated payload;
YY is hydroxyl, or at least one conjugated payload;
XX is hydroxyl, or at least one conjugated payload; and
wherein at least one of QQ, ZZ, YY, and XX is or at least one conjugated payload.

In certain cases, the glycoconjugates can include mixtures of the 2,6 and 2,3 linked oligosaccharides depicted above. In other embodiments, the glycoconjugates can be substantially only the 2,6 linked oligosaccharide, or substantially on the 2,3 linked oligosaccharide. In some embodiments, the glycoconjugate can be at least 90%, at least 95%, at least 98%, or at least 99% of the 2,6 linked oligosaccharide, while in other embodiments, the glycoconjugate can be at least 90%, at least 95%, at least 98%, or at least 99% of the 2,3 linked oligosaccharide.

In preferred embodiments, the GlcNAc residue can be bound to the CBA with a β-N-glycosidic linkage:

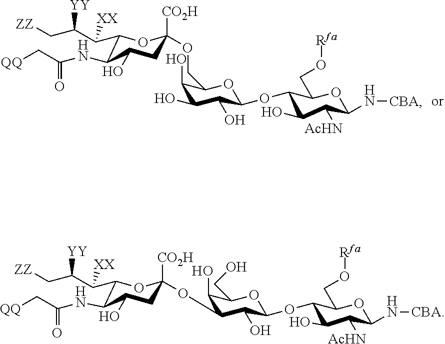

In other embodiments, the GlcNAc residue can be bound to the CBA with an α-N-glycosidic linkage.

When R^{fa} is a fucose moiety, it can be a fucose residue having the formula:

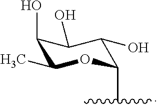

In certain embodiments, R^{fa} is hydrogen, while in other embodiments R^{fa} is fucose. In some embodiments, the compositions can have glycoconjugates in which at least 90%, at least 95%, at least 98%, or at least 99% of the glycoconjugates have a hydrogen atom for R^{fa} while in other embodiments, the compositions can have glycoconjugates can be at least 90%, at least 95%, at least 98%, or at least 99% of the glycoconjugates have a fucose residue for R^{fa}.

In certain embodiments, the cell-binding agent is an antibody, and the oligosaccharide is conjugated to the antibody through an asparagine side chain via a β-N-glycosidic bond:

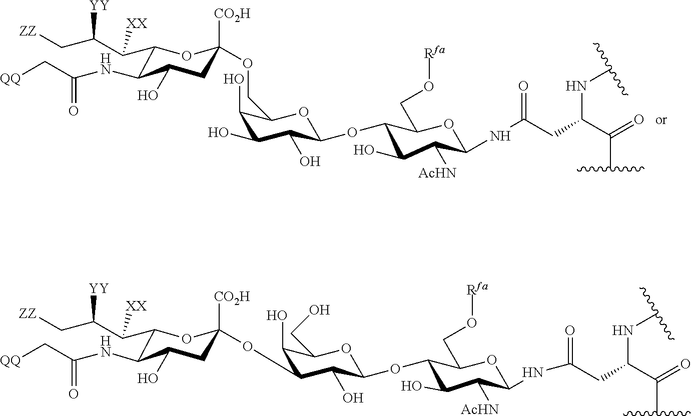

In some cases the GlcNAc moiety is conjugated to the antibody at the asparagine 297 (Asn297) residue according to the EU index as set forth in Kabat. In certain embodiments wherein y is 2, the GlcNAc moiety can be conjugated to both Asn297 residues in the Fc domain. In embodiments wherein y is 1, the GlcNAc moiety can be conjugated to one of the Asn297 residues in the Fc domain. When the antibody has been modified, either by chain elongation or truncation, the oligosaccharide can be conjugated to the asparagine residue corresponding to Asn297 in the unmodified antibody.

Provided herein are highly homogenous glycoconjugates, meaning that each individual CBA has the same glycan structures glycosylated to the CBA. For instance, in the case of antibodies, at least 75%, at least 80%, at least 85%, at least 90%, at least 95%, at least 97.5%, or at least 99% of the individual antibody molecules in the composition can have an identical glycan structure. For embodiments in which the oligosaccharide is glycosylated to Asn297, at least 75%, at least 80%, at least 85%, at least 90%, at least 95%, at least 97.5%, or at least 99% of the antibodies can be characterized by having the same glycan at Asn297.

The term “antibody” herein is used in the broadest sense and specifically covers monoclonal antibodies, polyclonal antibodies, dimers, multimers, multispecific antibodies {e.g., bispecific antibodies), and antibody fragments, so long as they exhibit the desired biological activity (Miller et al (2003) Jour, of Immunology 170:4854-4861). Antibodies may be murine, human, humanized, chimeric, or derived from other species. An antibody is a protein generated by the immune system that is capable of recognizing and binding to a specific antigen. (Janeway, C, Travers, P., Walport, M., Shlomchik (2001) ImmunoBiology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a fu II-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type/class (e.g. IgG, IgE, IgM, IgD, and IgA) or subtype/subclass (e.g. IgG1, lgG2, lgG3, lgG4, lgA1 and lgA2) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)2, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1 q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses”, e.g., lgG1, lgG2, lgG3, lgG4, IgA, and lgA2. The IgG isotype is preferred, in particular the IgG1 sub-type. The heavy-chain constant domains that correspond to the different classes of antibodies are called α, δ, ε, γ, and μ, respectively. The subunit structures and three-dimensional configurations of different classes of immunoglobulins are well known.

Techniques to reduce the in vivo immunogenicity of a non-human antibody or antibody fragment include those termed “humanization.”

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies. The expression “humanized antibody” also includes an immunoglobulin amino acid sequence variant or fragment thereof that comprises an FR having substantially the amino acid sequence of a human immunoglobulin and a CDR having substantially the amino acid sequence of a non-human immunoglobulin.

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g. murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins.

There are a range of humanization techniques, including ‘CDR grafting’, ‘guided selection’, ‘deimmunization’, ‘resurfacing’ (also known as ‘veneering’), ‘composite antibodies’, ‘Human String Content Optimization’ and framework shuffling.

The antibody may be an intact antibody. The antibody may be humanized, deimmunized or resurfaced. The antibody may be a fully human monoclonal IgG1 antibody, preferably IgG1,κ.

The numbering of the amino acids used herein is according to the numbering system of the EU index as set forth in Kabat et al. (1991, NIH Publication 91-3242, National Technical Information Service, Springfield, VA, hereinafter “Kabat”). The “EU index as set forth in Kabat” refers to the residue numbering of the human IgG 1 EU antibody as described in Kabat et al. supra.

In the case of substitutions in, for example, IgG2, IgG3, and IgG4 (or of igA1, lgA2, IgD, IgE, IgM etc.) the skilled person can readily use sequence alignment programs such as NCBI BLAST® (http://blast.ncbi.nlm.nih.gov/Blast.cgi) to align the sequences with IgG1 to determine which residues of the desired isoform correspond to the Kabat positions described herein.

In some embodiments the payload is conjugated to the N-linked glycan attached to an asparagine residue located at the position corresponding to 297 of IgG1 according to the EU index as set forth in Kabat.

In some embodiments the antibody is an intact antibody having 2 N-linked glycans bearing Sd(A)ₓ moieties (ie. y=2). In some embodiments the antibody has exactly 2 N-linked glycans bearing Sd(A)ₓ moieties.

In certain aspects, monoclonal antibodies that may be used in the conjugates and methods disclosed herein. Suitable monoclonal antibodies include abagovomab, abciximab, abituzumab, abrezekimab, abrilumab, actoxumab, adalimumab, adecatumumab, aducanumab, afasevikumab, afelimomab, alacizumab, alemtuzumab, alirocumab, altumomab, amatuximab, anatumomab mafenatox, andecaliximab, anetumab, anifrolumab, anrukinzumab, apolizumab, aprutumab, arcitumomab, ascrinvacumab, aselizumab, atezolizumab, atidortoxumab, atinumab, atorolimumab, avelumab, azintuxizumab, bapineuzumab, basiliximab, bavituximab, BCD-100, bectumomab, begelomab, belantamab, belimumab, bemarituzumab, benralizuma, fasenramab, berlimatoxumab, bermekimab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab, briakinumab, brodalumab, brolucizumab, brontictuzumab, burosumab, crysvitamab, cabiralizumab, camidanlumab, camrelizumab, canakinumab, cantuzumab mertansine, cantuzumab, caplacizumab, capromab, carlumab, carotuximab, catumaxomab, CBR96, cedelizumab, cemiplimab, cergutuzumab, certolizumab, cetrelimab, cetuximab, cibisatamab, cirmtuzumab, citatuzumab bogatox, cixutumumab, clazakizumab, clenoliximab, clivatuzumab, codrituzumab, cofetuzumab, coltuximab, conatumumab, concizumab, cosfroviximab, CR6261, crenezumab, crizanlizumab, crotedumab, cusatuzumab, dacetuzumab, daclizumab, dalotuzumab, dapirolizumab, daratumumab, dectrekumab, demcizumab, denintuzumab mafodotin, denosumab, depatuxizumab, derlotuximab, detumomab, dezamizumab, dinutuximab, diridavumab, domagrozumab, dorlimomab, dostarlimab, drozitumab, ds-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, hemlibra, enapotamab, enavatuzumab, enfortumab, enlimomab, enoblituzumab, enokizumab, enoticumab, ensituximab, epitumomab, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, fbta05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab, gevokizumab, gilvetmab, gimsilumab, girentuximab, glembatumumab, golimumab, gomiliximab, gosuranemab, guselkumab, ianalumab, ibalizumab, IBI308, ibritumomab, icrucumab, idarucizumab, ifabotuzumab, igovomab, iladatuzumab, IMAB362, imalumab, imaprelimab, imeiromab, imgatuzumab, inclacumab, indatuximab ravtansine, indusatumab vedotin, inebilizumab, infliximab, inolimomab, inotuzumab, intetumumab, IOMAB-B, ipilimumab, iratumumab, isatuximab, iscalimab, istiratumab, itolizumab, ixekizumab, keliximab, labetuzumab, lacnotuzumab, ladiratuzumab, lampalizumab, lanadelumab, landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab satetraxetan, lintuzumab, lirilumab, lodelcizumab, lokivetmab, loncastuximab, lorvotuzumab, losatuxizumab, lucatumumab, lulizumab, lumiliximab, lumretuzumab, lupartumab amadotin, lutikizumab, mapatumumab, margetuximab, marstacimab, maslimomab, matuzumab, mavrilimumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab, muromonab-CD3, nacolomab tafenatox, namilumab, naptumomab, naratuximab, namatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab, obiltoxaximab, obinutuzumab, ocaratuzumab, ocrelizumab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab, pintumomab, placulumab, plozalizumab, pogalizumab, polatuzumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab, ravagalimab, ravulizumab, raxibacumab, refanezumab, regavirumab, relatlimab, remtolumab, reslizumab, rilotumumab, rinucumab, risankizumab, rituximab, rivabazumab, robatumumab, roledumab, romilkimab, romosozumab, rontalizumab, rosmantuzumab, rovalpituzumab tesirine, rovelizumab, rozanolixizumab, ruplizumab, SA237, sacituzumab, samalizumab, samrotamab, sarilumab, satralizumab, satumomab, secukinumab, selicrelumab, seribantumab, setoxaximab, setrusumab, sevirumab, SGN-CD19a, SHP647, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, sulesomab, suptavumab, sutimlimab, suvizumab, suvratoxumab, tabalumab, tacatuzumab, tadocizumab, talacotuzumab, talizumab, tamtuvetmab, tanezumab, taplitumomab, tarextumab, tavolimab, tefibazumab, telimomab, telisotuzumab, tenatumomab, teneliximab, teplizumab, tepoditamab, teprotumumab, tesidolumab, tetulomab, tezepelumab, TGN1412, tibulizumab, tigatuzumab, tildrakizumab, timigutuzumab, timolumab, tiragotumab, tislelizumab, tisotumab, TNX-650, tocilizumab, tomuzotuximab, toralizumab, tosatoxumab, tositumomab, tovetumab, tralokinumab, trastuzumab, TRBS07, tregalizumab, tremelimumab, trevogrumab, tucotuzumab, tuvirumab, ublituximab, ulocuplumab, urelumab, urtoxazumab, ustekinumab, utomilumab, vadastuximab, vanalimabmab, vandortuzumab, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, and zolimomab.

The conjugate chemistry described herein above allows the glycosylated cell-binding agents described herein to be conjugated to a wide range of payloads. For example, in some embodiments the payload is, or comprises, a therapeutic agent (such as a therapeutic protein, lipid, or nucleic acid), a marker or imaging agent (such as radionuclide, fluorophore, or dye), a drug, an antibiotic, a vaccine, an immunosuppressant, an adjuvant, or a protective agent.

A preferred class of payload comprise a drug (also termed herein as a ‘drug moiety’), with the conjugation of the drug to the cell-binding agent allowing the drug to be delivered to the target cell with a high degree of precision.

The drug molecule may be a drug or a prodrug. In some embodiments, the drug is selected from the group consisting of pharmaceutically active compounds, in particular low to medium molecular weight compounds (e.g. about 200 to about 2500 Da, preferably about 300 to about 1750 Da). In some embodiments, the drug can be one or more of cytotoxins, immunomodulators, antiviral agents, antibacterial agents, peptides and oligonucleotides. Exemplary drugs include colchicine, vinca alkaloids, anthracyclines, camptothecins, doxorubicin, daunorubicin, taxanes, pyridinobenzodiazepines (PDDs), calicheamycins and other ene-diyne compounds, tubulysins, exatecans, irinotecans, inhibitory peptides, amanitin, deBouganin, duocarmycins, maytansines or auristatins, vinca alkaloids, anthracyclines, taxanes, amanitin, in particular vinca alkaloids, anthracyclines, camptothecins, taxanes, tubulysins, amanitin, maytansines and auristatins.

For the avoidance of doubt, the drug may not be a pyrrolobenzodiazepine (PBD), i.e., a compound including the following substructure:

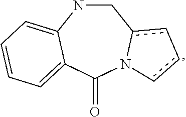

where any atom may be further substituted with any functional group.

In preferred embodiments, the drug moiety is conjugated to the glycosylated cell-binding agents described herein via a linker moiety (a so-called ‘drug-linker’ payload) to yield a conjugate in which one or more drugs are linked to the same sialoside, having the formula:

[[Drug-linker]_{z}-sialoside-Gal-GlcNAc]_{z}-CBA,

wherein the one or more of positions QQ, XX, YY, and ZZ on the sialoside as defined above are linker-payloads, and z is in each case independently selected from 1, 2, 3, 4, 5, 6, 7, or 8.

In some embodiments, multiple drugs can be conjugated to the same linker that is conjugated to the sialoside, having the formula:

[[Drug]_{z}-linker-sialoside-Gal-GlcNAc]_{z}-CBA.

In other embodiments, multiple drugs can be conjugated to the same linker, and multiple linkers can be conjugated to the sialoside, having the formula:

[[[Drug]_{z}-linker]_{z}-sialoside-Gal-GlcNAc]_{z}-CBA.

In certain embodiments, the linker can include a ring system obtained from a cycloaddition reaction between a 1,3 dipole and a strained cycloalkyne or strained trans cycloalkene.

Accordingly, in some exemplary embodiments the payload is linked to the sialoside at position QQ, thus:

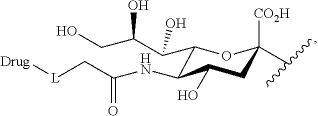

wherein L is a linker. In some exemplary embodiments the payload is linked to the sialoside at position ZZ, thus:

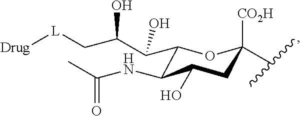

In yet further embodiments, the payload (e.g., drug) is linked to the sialoside at position QQ and position ZZ. The payload and linkers may be the same or different.

A variety of cytotoxic compounds may serve as the payload. In some embodiments, the payload includes a DNA damaging agent, an inhibitor of tubulin polymerization (which may also be called microtubule inhibitor or microtubule destabilizers, a topoisomerase inhibitor, a RNA splicing inhibitor, or a RNA polymerase inhibitor. As used herein, DNA damaging agent does not include a pyrrolobenzodiazepine compound, which is defined above. In some embodiments, the conjugates can include at least two different agents drawn from the above classes. For instance, the conjugates can include multiple DNA damaging agents or multiple microtubule inhibitors. In some cases the conjugates can include at least one DNA damaging agent and at least one microtubule inhibitor. In other embodiments, the conjugates can include at least one DNA damaging agent and at least one topoisomerase inhibitor. In further embodiments, the conjugates can include at least one microtubule inhibitor and at least one topoisomerase inhibitor. In yet other embodiments, the conjugates can include at least one microtubule inhibitor, at least one DNA damaging agent, and at least one topoisomerase inhibitor.

Suitable DNA damaging agents include enediyne compounds, doxorubicin compounds, duocarmycin compounds, Suitable microtubule inhibitors include dolastatin compounds, taxanes, vinca alkaloids, maytansine compounds, tubulysin compounds, eribulin compounds, and crytophycin compounds. Suitable topoisomerase inhibitors include camptothecin compounds and lamellarin compounds.

Other types of payloads may also be conjugated, including immune stimulating agents.

In some embodiments the payload can include an enediyne antitumor antibiotics, of which exemplary members include the calicheamicins, shishijimicins, uncialamycin, neocarzinostatins, esperamicins, dynemicins, and golfomycins.

In some instances the payload can include a calicheamicin compound having the formula:

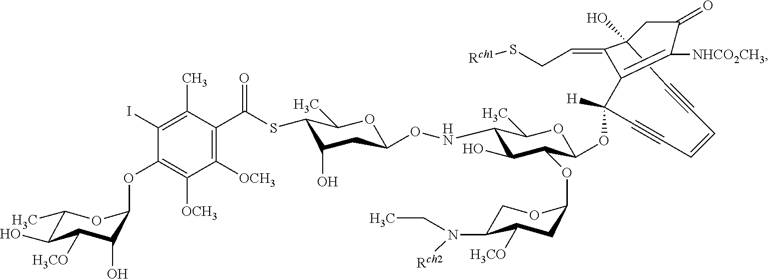

wherein one of R^{ch1} or R^{ch2} is a linker conjugated to the terminal sialoside. When not a linker, R^{ch1} can be H, —S—S—CH₃, C(O)CH₃, or any sufficiently labile group that can be cleaved to give the free thiol. When not a linker, R^{ch2} can be H, C₁₋₄alkyl, or C(O)C₁₋₄alkyl.

In some instances, the payload can include a shishijimicins compound having the formula:

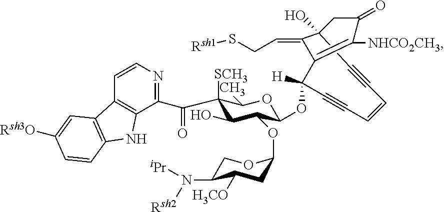

wherein one of R^{sh1}, R^{sh2}, or R^{sh3} is a linker conjugated to the terminal sialoside. When not a linker, R^{sh1} can be H, —S—S—CH₃, C(O)CH₃, or any sufficiently labile group that can be cleaved to give the free thiol. When not a linker, R^{sh2} can be H, C₁₋₄alkyl, or C(O) C₁₋₄alkyl. When not a linker, R^{sh3} can be H.

In some instances, the payload can include a uncialamycin compound having the formula:

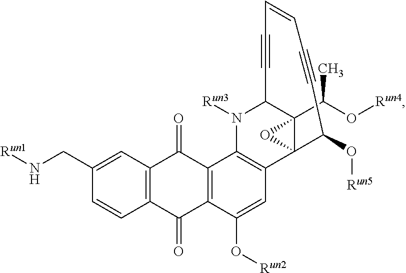

wherein one of R^{un1}, R^{un2}, R^{un3}, R^{un4}, or R^{un5} is a linker conjugated to the terminal sialoside, and the remainder are H.

In some instance, the payload can include a neocarzinostatin compound having the formula:

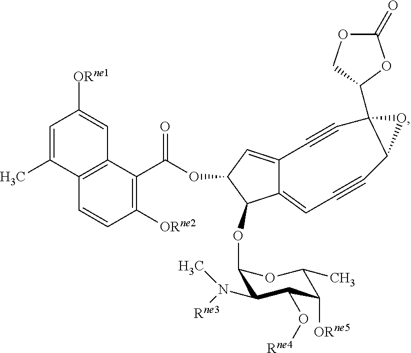

wherein one of R^{ne1}, R^{ne2}, R^{ne3}, R^{ne4}, or R^{ne5} is a linker conjugated to the terminal sialoside and the remainder are independently H or C₁₋₄alkyl.

In some instances the payload can include a esparamicin compound having the formula:

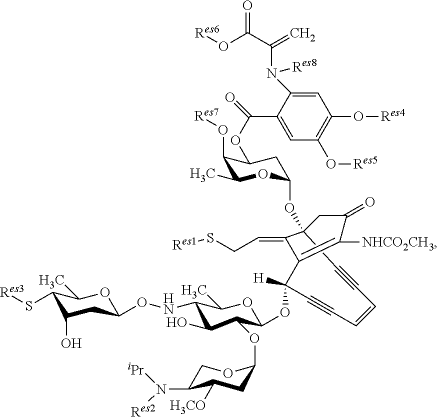

wherein one of R^{es1}, R^{es2}, R^{es3}, R^{es4}, R^{es4}, R^{e5}, R^{es6}, R^{es7}, or R^{es8} is a linker conjugated to the terminal sialoside. When not a linker, R^{es1} can be H, —S—S—CH₃, C₁₋₄alkyl, C(O)CH₃, or any sufficiently labile group that can be cleaved to give the free thiol. When not a linker, R^{es2}, R^{es3}, R^{es4}, R^{es4}, R^{es5}, R^{es6}, R^{es7} and R^{es8} are independently H, C₁₋₄alkyl, or C(O)C₁₋₄alkyl.

In some instances the payload can be a dynemycin compound having the formula:

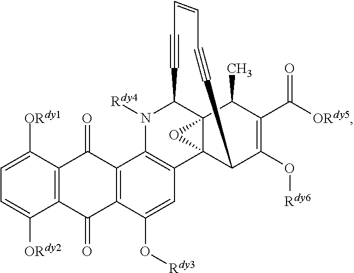

wherein one of R^{dy1}, R^{dy2}, R^{dy3}, R^{dy4}, R^{dy4}, R^{dy5}, or R^{dy6} is a linker conjugated to the terminal sialoside and the remainder are independently H or C₁₋₄alkyl.

In other instances the payload can include a golfomycin compound having the formula:

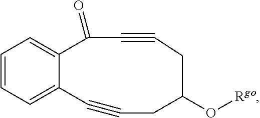

wherein R^{go} is a linker conjugated to the terminal sialoside.

In some embodiments, the payload can be a dolastatin compound, for instance, a dolastatin 10 or dolastatin 15 analog. In certain embodiments, the dolastatin compound can be an auristatin, for instance having the formula:

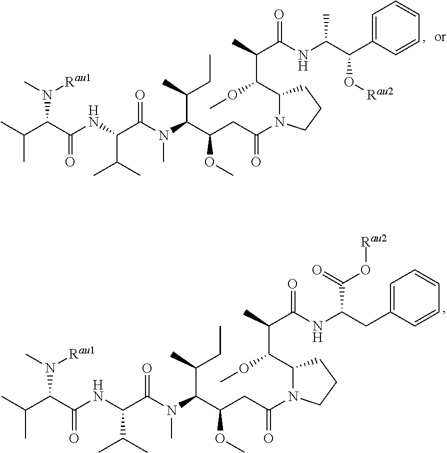

wherein one of R^{au1} and R^{au2} is a linker conjugated to a terminal sialoside, and the other is selected from H, and C₁₋₄alkyl.

In other embodiments, the payload can include a daunorubicin or doxorubicin compound having the formula:

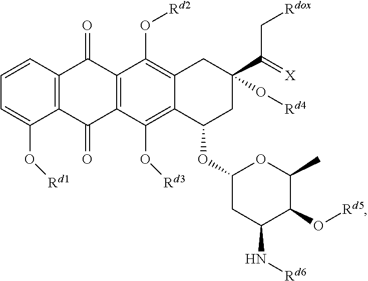

wherein R^{dx} is either H or OR^{d7}, X is either O or NR^{d8} and wherein one of R^{d1}, R^{d2}, R^{d3}, R^{d4}, R^{d4}, R^{d5}, R^{d6}, R^{d7}, or R^{d8} is a linker conjugated to the terminal sialoside, and the remainder are independently selected from H and C₁₋₄alkyl.

In other cases the payload can include an ansamitocin derivative, for instance a mertansine compound having the formula:

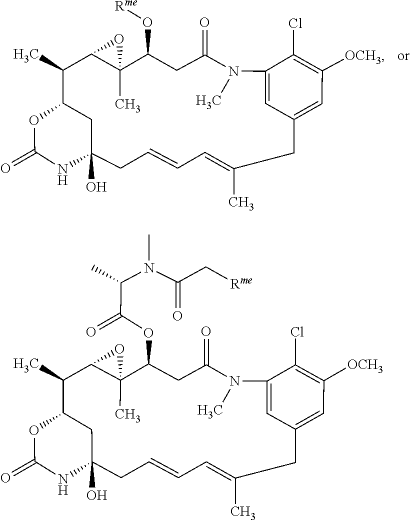

wherein R^{me} is a linker conjugated to a terminal sialoside.

In further embodiments, the payload can include a vinca alkaloid, for instance a vincristine/vinblastine compound having the formula:

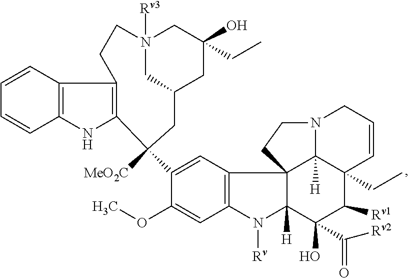

wherein R^{v} is CH₃ or C(O)H;
R^{v1} is OH, OAc, or a linker conjugated to the terminal sialoside;
R^{v2} is OH, OCH₃, or a linker conjugated to the terminal sialoside; and
R^{v3} is absent or a linker conjugated to the terminal sialoside.

In other embodiments, the payload can include an eribulin compound having the formula:

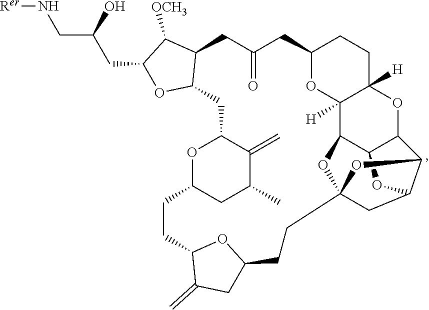

wherein R^{er} is a linker conjugated to the terminal sialoside.

In other embodiments, the payload can include a camptothecin compound having the formula:

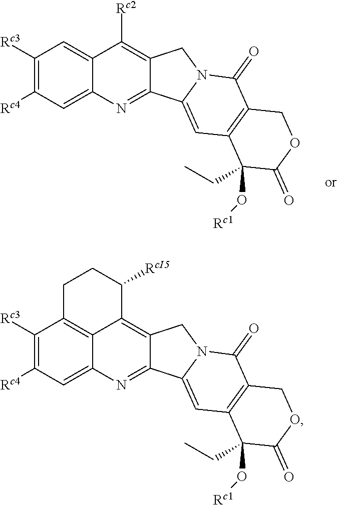

wherein R^{c1} is H or a linker to the terminal sialoside;
R^{c2} is H, CH₂CH₃, or a linker to the terminal sialoside;
R^{c3} is H, OH, CH₃; or a linker to the terminal sialoside;
R^{c4} is H or F; and
R^{c5} is NH₂ or a linker to the terminal sialoside.

Other compounds that can be included in the payload are duocarmycin compounds having the formula:

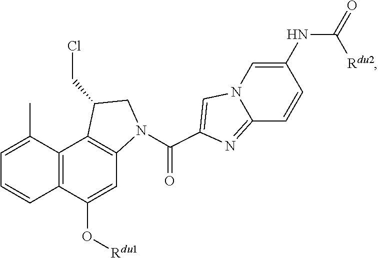

wherein R^{du1} is H or a linker to the terminal sialoside; and
R^{du2} is C₁₋₈alkyl, OC₁₋₈alkyl phenyl, 4-hydroxyphenyl, or a linker to a terminal sialoside.

In some embodiments, the payload includes a taxanes compound having the formula:

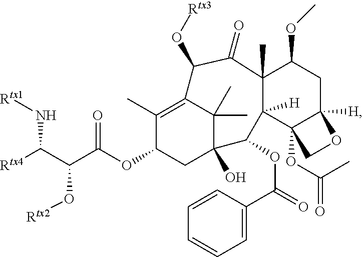

wherein R^{tx1} is —C(O)Ph, —C(O)OtBu, or a linker to the terminal sialoside;
R^{tx2} is H or a linker to the terminal sialoside;
R^{tx3} is —C(O)CH₃, OMe, or a linker to the terminal sialoside;
R^{t4} is phenyl or —CH═C(CH₃)₂.

In some embodiments, the payload includes a cryptophycin compound having the formula

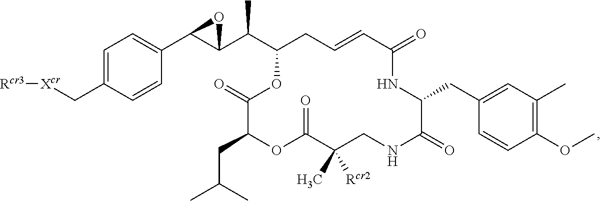

### Wherein X^{cr} is O or NH;

R^{CR2} is H or CH₃; and
R^{cr3} is a linker to the terminal sialoside.

In some instances the payload can include other tubulin inhibitors such as hemiasterlin, HTI-286), colchicine, discodermolide, taccalonolide A, taccalonolide B, taccalonolide AF, taccalonolide AJ, taccalonolide AI-epoxide, laulimalide, epothilone A or epothilone B.

Linkers bind the payload with the terminal sialoside, which in some embodiments may be depicted as follows:

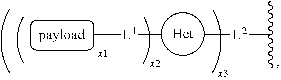

wherein payload is as defined above, Het represents a heterocyclic system, L¹ is selected from null or sublinker from Het to payload, L² is selected from null or sublinker from Het to sialoside, x1 is an integer selected from 1, 2, 3, 4, 5, 6, 7, or 8; x2 is an integer selected from 1, 2, 3, 4, 5, 6, 7, or 8; and x3 is an integer selected from 1, 2, 3, 4, 5, 6, 7, or 8. For embodiments in which one linker includes multiple payloads (i.e., at least one of x1, x2, or x3>1), the payloads can be the same or different.

For embodiments featuring multiple linker-payload moieties attached to the same sialoside (i.e., at least two of QQ, XX, YY, and ZZ are conjugated payloads), the identities of the payloads, Het, L¹, L², x1, x2, and x3 can be the same or different.

Exemplary heterocycle systems include fused polycyclic heterocycle systems.

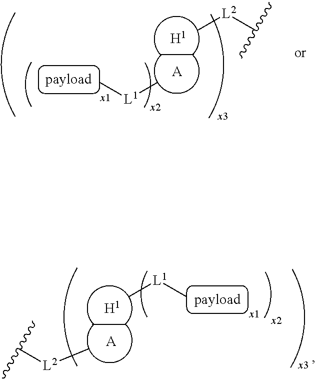

wherein H¹ represents a heterocyclic ring, and A represents a carbocyclic or heterocyclic ring, preferably a ring having 8 atoms in the ring skeleton. Exemplary 8-atoms rings include cyclooctane, cyclooctene, aza-cyclooctane, aza-cyclooctene, 2-azacyclooctanone and unsaturated derivatives thereof. In some embodiments the 8-atom ring can be fused to one or more aromatic rings.

The heterocyclic ring represented by H¹ may be formed from cycloaddition reaction between (a) either a 1,3 dipole or 1,2,4,5 tetrazine and (b) either a strained alkyne or strained alkene. Preferred strained alkynes include cyclooctyne and preferred strained alkenes include trans-cyclooctene. Heterocyclic rings include, but are not limited to, triazoles, 1,2 pyridazines, oxazoles, isooxazoles, oxadiazoles, and saturated and partially unsaturated analogs of such rings.

In some embodiments, e.g., (x2 and x3=1), the heterocycle system can have the formula:

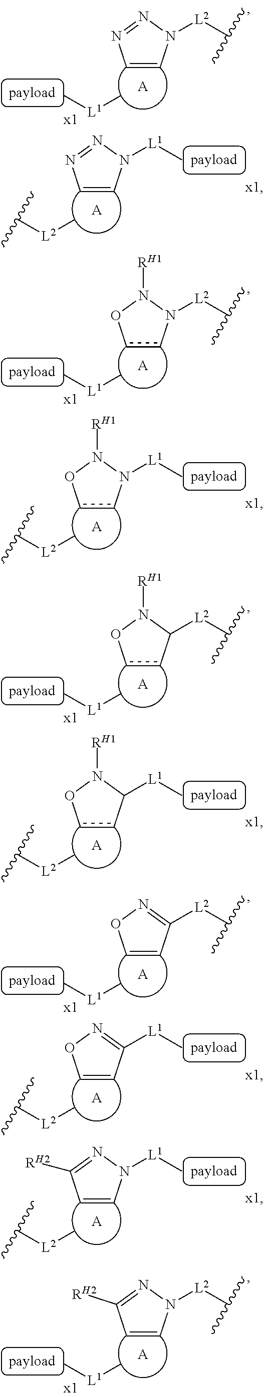

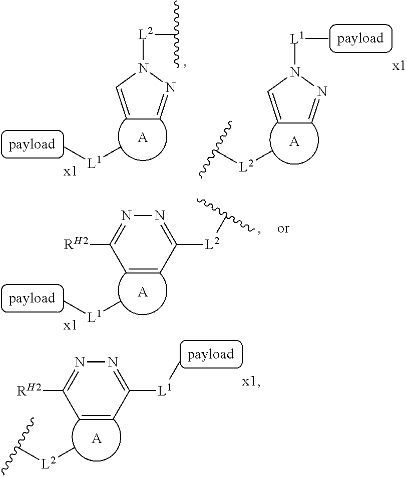

wherein x is as defined above, R^{H1} is selected from H, C₁₋₄alkyl, aryl, C₁₋₄alkaryl, and may together with L¹ or L² form a ring;
R^{H2} is selected from H, C₁₋₄alkyl, aryl, C₁₋₄alkaryl, and [symbol] represents a single or double bond.

In certain embodiments, the A ring can have the formula:

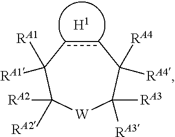

wherein R^{A1}, R^{A1′}, R^{A2}, R^{A2′}, R^{A3}, R^{A3′}, R^{A4}, and R^{A4′} are independently selected from null, H, F, Cl, Br, I, C₁₋₄alkyl, C₁₋₄alkoxy, aryl; and wherein any one of R^{A1}, R^{A1′}, R^{A2}, R^{A2′}, R^{A3}, R^{A3′}, R^{A4}, and R^{A4′} can be L¹ or L²; wherein any two or more of R^{A1}, R^{A1′}, R^{A2}, R^{A2′}, R^{A3}, R^{A3′}, R^{A4}, and R^{A4′} can together form a ring; for instance R^{A1} and R^{A2}, as well as R^{A3} and R^{A4} and can each together form an aromatic ring, while R^{A1′}, R^{A2′}, R^{A3′}, and R^{A4′} are each null;

When one of R^{A1}, R^{A1′}, R^{A2}, R^{A2′}, R^{A3}, R^{A3′}, R^{A4}, and R^{A4′} is L¹ or L², W can be CH₂CH₂. When none of R^{A1}, R^{A1′}, R^{A2}, R^{A2′}, R^{A3}, R^{A3′}, R^{A4}, and R^{A4′} are L¹ or L², W can be a group having the formula:

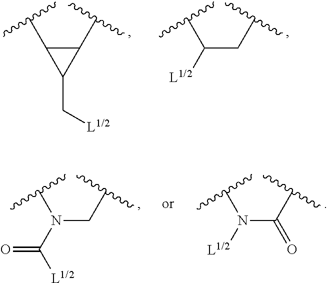

wherein L^{1/2} represents either L¹ or L².
with the proviso that when one of W, R^{A1}, R^{A1′}, R^{A2}, R^{A2′}, R^{A3}, R^{A3′}, R^{A4}, and R^{A4′} includes L¹, none of W, R^{A1}, R^{A1′}, R^{A2}, R^{A2′}, R^{A3}, R^{A3′}, R^{A4}, and R^{A4′} includes L²; and when one of W, R^{A1}, R^{A1′}, R^{A2}, R^{A2′}, R^{A3}, R^{A3′}, R^{A4}, and R^{A4′} includes L², none of W, R^{A1}, R^{A1′}, R^{A2}, R^{A2′}, R^{A3}, R^{A3′}, R^{A4}, and R^{A4′} includes L¹.

In some embodiments, the A ring can have the formula:

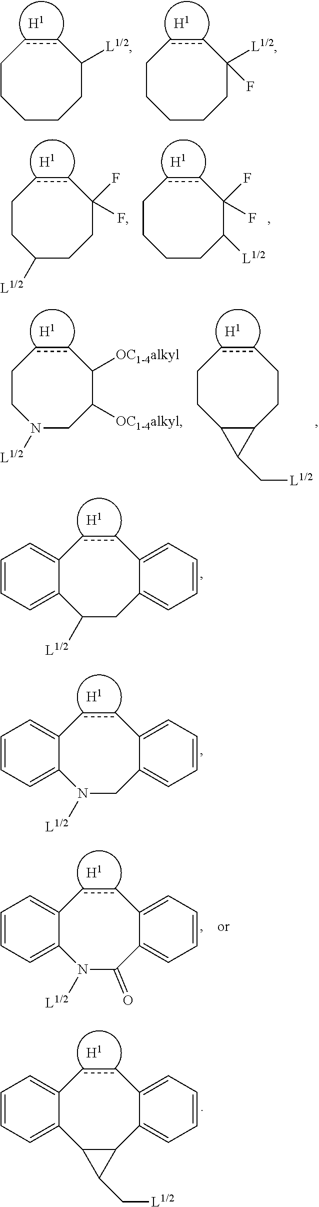

Although not depicted above, it is understood that if L¹ is connected to 8-atom ring, L² will be connected to the Heterocycle, and vice versa.

In some embodiments, L² can be null, or a group having the formula:

-L2¹-L2²-L2³-L2⁴-L2⁵-L2⁶-,

wherein:
L2¹ is bonded to the heterocyclic system and is selected from null, C₁₋₈alkylene, arylene, heteroaryl, heterocyclyl, poly(ethylene), poly(acetal); poly(glycerol), S, O, NR²¹, OC(═O), OC(═O)NR²¹, NR²¹C(═O), NR²¹C(═O)O, NR²¹C(═O), NR²¹C(═O)NR²¹, OC(═O)O, wherein R²¹ is in each case selected from H and C₁₋₄alkyl;
L2² is selected from null, C₁₋₈alkylene, arylene, heteroaryl, heterocyclyl, poly(ethylene), poly(acetal); poly(glycerol), S, O, NR²², OC(═O), OC(═O)NR²², NR²²C(═O), NR²²C(═O), NR²²C(═O)O, NR²²C(═O)NR²², OC(═O)O, wherein R²² is in each case selected from H and C₁₋₄alkyl;
L2³ is selected from null, C₁₋₈alkylene, arylene, heteroaryl, heterocyclyl, S, poly(ethylene), poly(acetal); poly(glycerol), O, NR²³, OC(═O), OC(═O)NR²³, NR²³C(═O), NR²³C(═O), NR²³C(═O)O, NR²³C(═O)NR²³, OC(═O)O, wherein R²³ is in each case selected from H and C₁₋₄alkyl;
L2⁴ is selected from null, C₁₋₈alkylene, arylene, heteroaryl, heterocyclyl, poly(ethylene), poly(acetal); poly(glycerol), S, O, NR²⁴, OC(═O), OC(═O)NR²⁴, NR²⁴C(═O), NR²⁴C(═O), NR²⁴C(═O)O, NR²⁴C(═O)NR²⁴, OC(═O)O, wherein R²⁴ is in each case selected from H and C₁₋₄alkyl;
L2⁵ is selected from null, C₁₋₈alkylene, arylene, heteroaryl, heterocyclyl, poly(ethylene), poly(acetal); poly(glycerol), S, O, NR²⁵, OC(═O), OC(═O)NR²⁵, NR²⁵C(═O), NR²⁵C(═O), NR²⁵C(═O)O, NR²⁵C(═O)NR²⁵, OC(═O)O, wherein R²⁵ is in each case selected from H and C₁₋₄alkyl;
L2⁶ is bonded to the sialoside and is selected from null, C₁₋₈alkylene, arylene, heteroaryl, heterocyclyl, poly(ethylene), poly(acetal); poly(glycerol), S, O, NR²⁶, OC(═O), OC(═O)NR²⁶, NR²⁶C(═O), NR²⁶C(═O), NR²⁶C(═O)O, NR²⁶C(═O)NR²⁶, OC(═O)O, wherein R²⁶ is in each case selected from H and C₁₋₄alkyl.

In certain embodiments, any two or more of L2¹, L2², L2³, L2⁴, L2⁵, and L2⁶ can together form a ring.

The skilled person understands that selection of null for each of L2¹, L2², L2³, L2⁴, L2⁵, and L2⁶ produces an embodiment in which L² is null.

In certain embodiments, L2⁶ is NHC(O)NH, while in other embodiments, L2⁶ is heterocyclyl or heteroaryl, for instance a triazole, a 1,2 pyridazine, an oxazole, an isooxazole, an oxadiazole, and saturated and partially unsaturated analogs thereof.

In certain embodiments, L2¹ is arylene, for instance 1,4-phenylene.

In some embodiments, L2¹ is null, OC(O)NH, C₁₋₈alkylene, preferably C₁₋₃alkylene or arylene, for instance 1,4-phenylene, L2² is null or C₁₋₈alkylene, preferably C₁₋₃alkylene, L2³ is null, C(═O)NH, NHC(═O), NHC(═O)O, or OC(═O)NH; L2⁴ is null or poly(ethylene), L2⁵ is null or C₁₋₈alkylene, preferably C₁₋₃alkylene, and L2⁶ is null or heterocyclyl or heteroaryl, for instance a triazole, a 1,2 pyridazine, an oxazole, an isooxazole, an oxadiazole, and saturated and partially unsaturated analogs thereof.

In certain embodiments, L2¹ is OC(O)NH, and each of L2², L2³, L2⁴, L2⁵, and L2⁶ is null.

By way of example, certain selections for x, L2¹, L2², L2³, L2⁴, L2⁵, and L2⁶ will produce embodiments having the following partial structures:

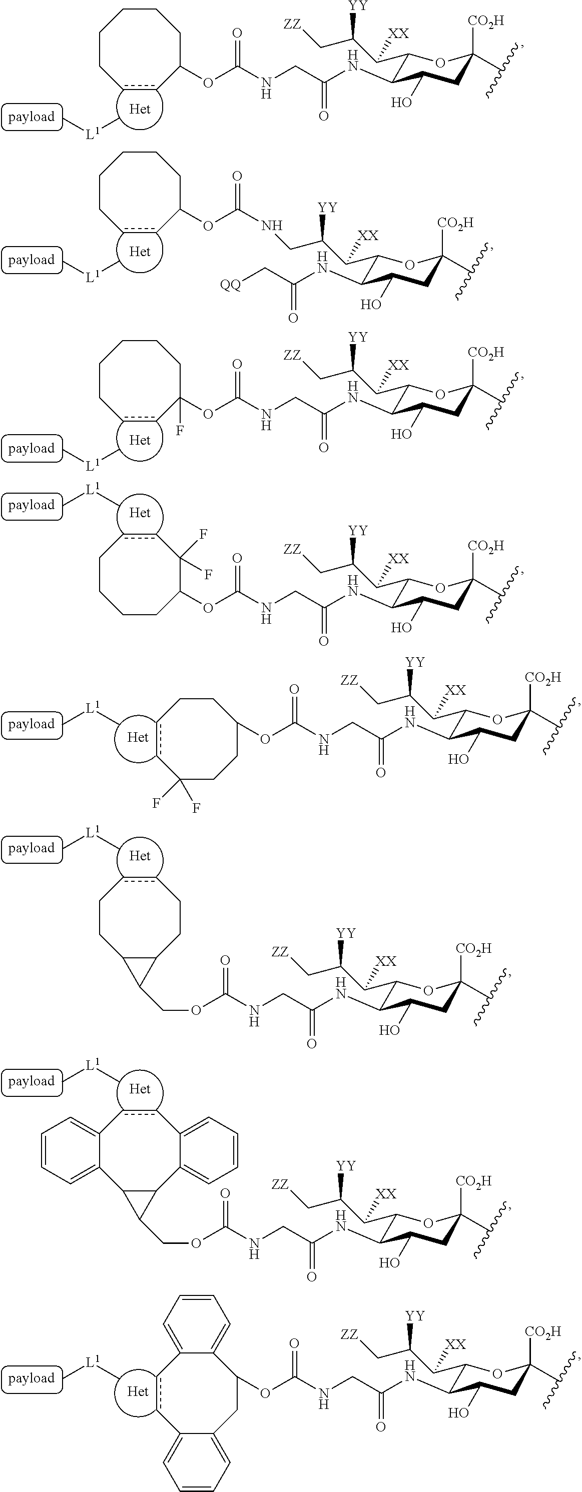

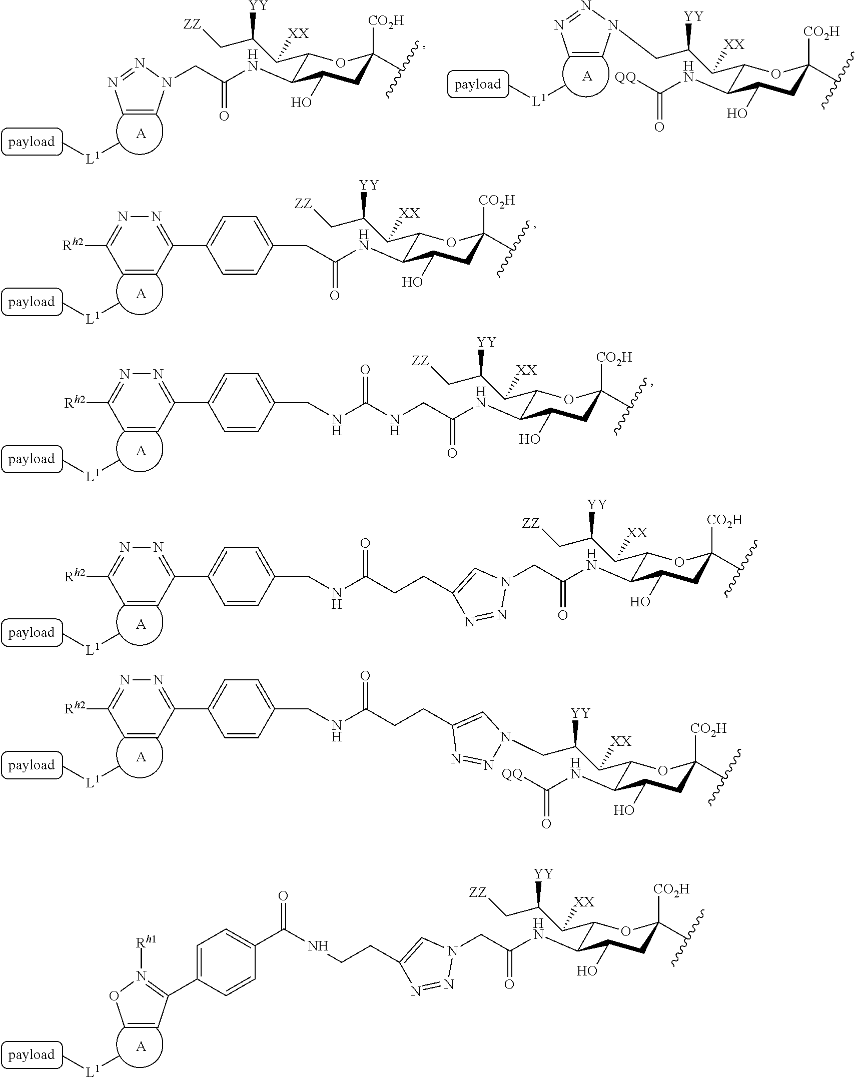

wherein L¹, payload, R^{h2}, A, QQ, ZZ, YY, and XX are as defined above. As aforementioned, more than one of QQ, ZZ, YY, and XX can be a conjugated payload, having the same of different payload, and the same or different linker.

In some embodiments, L¹ can be null, or a group having the formula:

-L1¹-L1²-L1³-L1⁴-L1⁵-L⁶-,

wherein:
L1¹ is bonded to the heterocyclic system and is selected from null, C₁₋₈alkylene, arylene, heteroaryl, heterocyclyl, poly(ethylene), poly(acetal); poly(glycerol), S, O, NR¹¹, OC(═O), OC(═O)NR¹¹, NR¹¹C(═O), NR¹¹C(═O), NR¹¹C(═O)NR¹¹, OC(═O)O, wherein R¹¹ is in each case selected from H and C₁₋₄alkyl;
L1² is selected from null, C₁₋₈alkylene, arylene, heteroaryl, heterocyclyl, poly(ethylene), poly(acetal); poly(glycerol), S, O, NR¹², OC(═O), OC(═O)NR¹², NR¹²C(═O), NR¹²C(═O), NR¹²C(═O)NR¹², OC(═O)O, wherein R¹² is in each case selected from H and C₁₋₄alkyl;
L1³ is selected from null, C₁₋₈alkylene, arylene, heteroaryl, heterocyclyl, poly(ethylene), poly(acetal); poly(glycerol), S, O, NR¹³, OC(═O), OC(═O)NR¹³, NR¹³C(═O), NR¹³C(═O), NR¹³C(═O)NR¹³, OC(═O)O, wherein R¹³ is in each case selected from H and C₁₋₄alkyl;
L1⁴ is selected from null, C₁₋₈alkylene, arylene, heteroaryl, heterocyclyl, poly(ethylene), poly(acetal); poly(glycerol), S, O, NR¹⁴, OC(═O), OC(═O)NR¹⁴, NR¹⁴C(═O), NR¹⁴C(═O), NR¹⁴C(═O)NR¹⁴, OC(═O)O, wherein R¹⁴ is in each case selected from H and C₁₋₄alkyl;
L1⁵ is selected from null, C₁₋₈alkylene, arylene, heteroaryl, heterocyclyl, poly(ethylene), poly(acetal); poly(glycerol), S, O, NR¹⁵, OC(═O), OC(═O)NR¹⁵, NR¹⁵C(═O), NR¹⁵C(═O), NR¹⁵C(═O)NR¹⁵, OC(═O)O, wherein R¹⁵ is in each case selected from H and C₁₋₄alkyl;
L1⁶ is bonded to the payload and is selected from null, C₁₋₈alkylene, arylene, heteroaryl, heterocyclyl, poly(ethylene), poly(acetal); poly(glycerol), S, O, NR¹⁶, OC(═O), OC(═O)NR¹⁶, NR¹⁶C(═O), NR¹⁶C(═O), NR¹⁶C(═O)NR¹⁶, OC(═O)O, wherein R¹⁶ is in each case selected from H and C₁₋₄alkyl.

In certain embodiments, any two or more of L1¹, L1², L1³, L1⁴, L1⁵, and L1⁶ can together form a ring.

L1³ can be a branched C₁₋₈alkylene, arylene, heteroaryl, or heterocyclyl group. By way of example, L1³ can be a phenyl group having the formula:

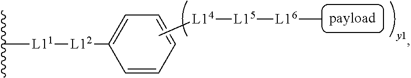

wherein y1 is any substitution number permitted by valence. In the exemplary formula above, x1 can be 1, 2, 3, 4, or 5. The skilled person recognizes that other possible L1³ groups will give rise to different x1 possibilities. In other instances, L1³ can be a branched alkylene, e.g., a methylene having the formula:

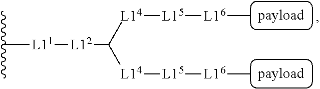

or a methine having the formula:

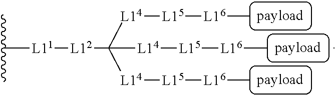

In some embodiments, L1³ can include a polymeric group, for instance a poly(glycerol) having the formula:

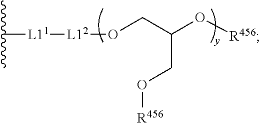

or
a polyacetal having the formula:

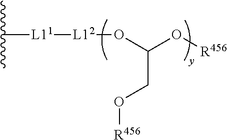

wherein y is from 1-1,000; and
R⁴⁵⁶ is selected from hydrogen or a moiety of Formula (456):

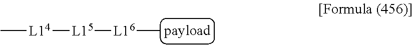

and the number of times that R⁴⁵⁶ is the moiety of Formula 456 is less than 30.

A cleavable L¹ group will include at least one functional group that undergoes bond-breaking under environmental conditions. Cleavable groups include acid-sensitive groups, redox sensitive groups, and enzyme-cleavable groups, for instance, protease cleavable groups. Exemplary acid-sensitive groups include Schiff bases/imines, hydrazones, boronic esters, and acetals. Exemplary redox-sensitive groups include thioacetals, oxalate esters, disulfides, peptides, and diselenide groups. Exemplary enzyme cleavable groups include peptide fragments Val-Lys, Val-Ala, Val-Arg, Phe-Lys, and Val-Cit.

In some instances, L¹ can include a self-immolating spacer. A self-immolative spacer refers to a chemical moiety bonded to a selectively cleavable group, wherein activation of the cleavable group results in a cascade of reactions that ultimately liberates the payload from the spacer. Exemplary self-immolative spacers include p-aminobenzyl alcohols, p-hydroxybenzyl alcohols, 2-aminoimidazol-5-methanol moieties, ortho- or para-aminobenzylacetals, aminobutyric acid amides, 1,2 diamino ethylene, 1,3 diaminopropylene

In some embodiments, L¹ can include a self-immolative spacer, cleavable group, and optional additional linker, e.g., a conjugate having the formula:

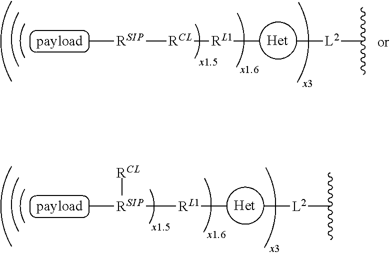

wherein R^{SIP} is one or more self-immolative spacers, R^{CL} is a cleavable group, and R^{L1}, when present, is an additional linker, x1.5 is an integer selected from 1, 2, 3, 4, 5, 6, 7, and 8; and x1.6 is an integer selected from 1, 2, 3, 4, 5, 6, 7, and 8.

When the payload includes a functionalizable amine or alcohol group, the payload can be bonded to L¹ or R^{SIP} via a carbamate, carbonate, phosphonate or sulfonate group, e.g.,

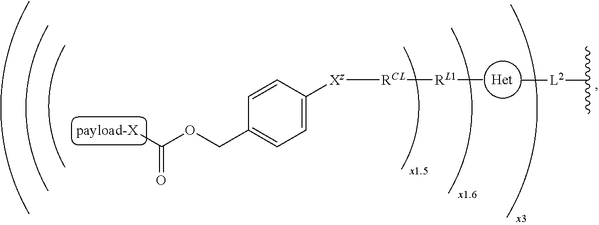

In the embodiments depicted below, x1.5, x1.6, and x3 are all selected to be 1. Also contemplated within the scope of the invention are embodiments in which those variables are greater than 1.

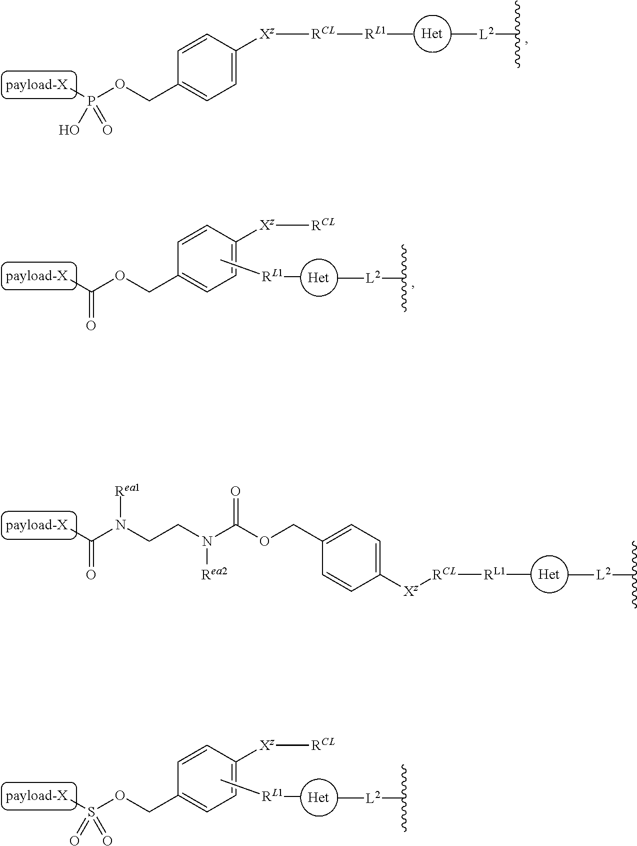

X is an oxygen or nitrogen atom in the payload, and X^{z} is O, NH, or NC₁₋₄alkyl. Benzyl self-immolating spacers depicted above may be further substituted one or more times by electron withdrawing groups like nitro, fluoro, trifluoromethyl, and the like. R^{ea1} and R^{ea2} can be independently selected from H, C₁₋₄alkyl, or (CH₂CH₂O)ₙCH₂CH₂OH, wherein n is from 0, 1, 2, or 3.

By way of example, the auristatin family (structure partially depicted) of payloads can be conjugated through the secondary amine functional group (that is, R^{au1} forms the linker and R^{au2} is hydrogen):

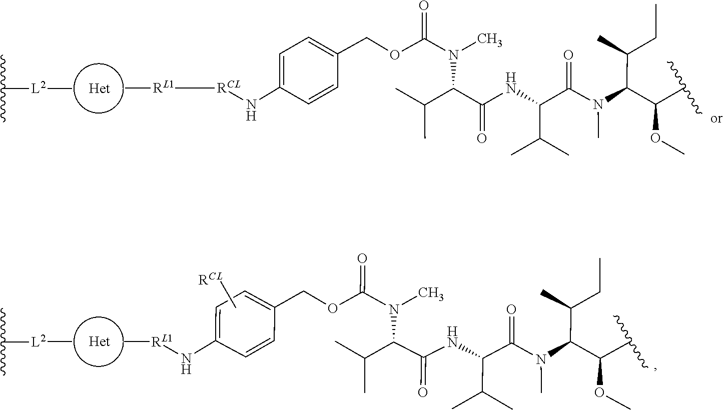

In some cases R^{CL} is a peptidyl residue, e.g.,

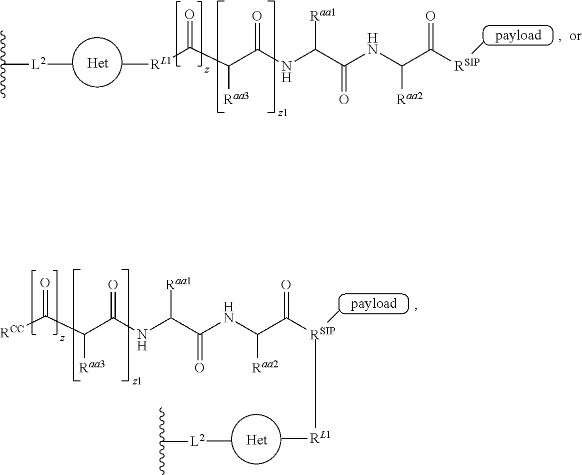

wherein z is 1 or 0, z1 is 1 or 0, R^{CC} is H, peptidyl, C₁₋₆alkyl, cycloalkyl, aryl, heteroaryl, or heterocyclyl, R^{aa1}, R^{aa2}, and R^{aa3} are independently selected from H, C₁₋₆alkyl optionally substituted with phenyl, COOH, NH₂, COHNH₂, NHC(O)NH₂. In certain embodiments z1 is 0 and R^{aa1} is isopropyl and R^{aa2} is (—CH₂)₄NH₂, (—CH₂)₃NHC(O)NH₂, (—CH₂)₃NHC(NH)NH₂, or CH₃. In other cases z1 is 0 and R^{aa1} is benzyl and R^{aa2} is (—CH₂)₄NH₂. In yet further embodiments, z1 is 1, R^{aa1} is isopropyl, R^{aa2} is (—CH₂)₃NHC(O)NH₂, and R^{aa3} is (—CH₂)COOH.

The peptidyl residues may have the following formula:

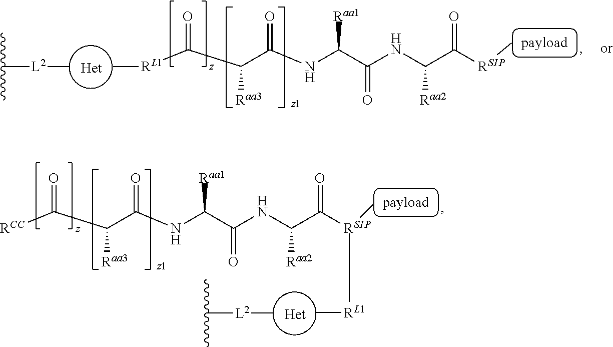

wherein R^{cc}, z, z1, R^{aa1}, R^{aa2}, R^{aa3}, R^{SIP} are as defined above.

In some instances, R^{SIP} can be a 4-aminobenzyl alcohol having the formula, exemplified below when z1 is 0:

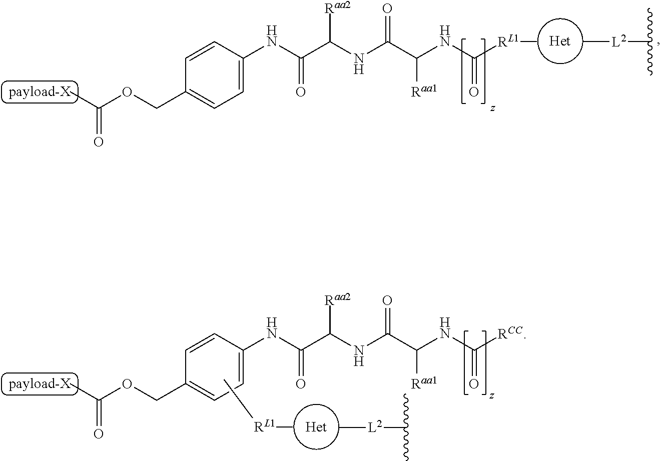

R^{SIP} can be a 4-aminobenzyl alcohol when z1 is 1.

In some instances, R^{CL} is a peptide group having the formula:

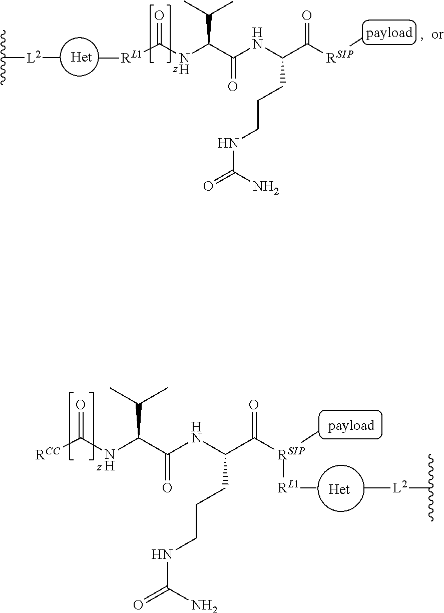

In other embodiments R^{CL} can be a gluconic acid residue, for instance:

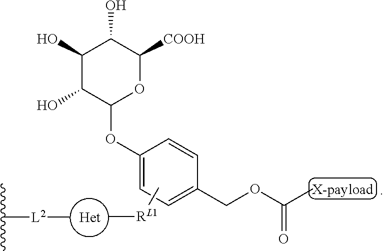

In other embodiments, the cleavable group can be a disulfide:

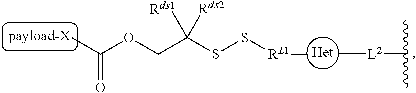

wherein R^{ds1} and R^{ds2} are independently selected from H and C₁₋₄alkyl. In some instances, R^{ds1} and R^{ds2} are both hydrogen, or R^{ds1} and R^{ds2} are both methyl. In other instances R^{ds1} is hydrogen and R^{ds2} is C₁₋₄alkyl.

When the payload includes a ketone or aldehyde, the cleavable group can include a hydrazone:

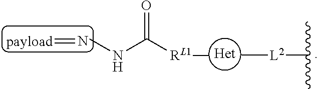

Hydrolysis of the hydrazone is believed to liberate the payload with its original carbonyl. By way of example, a hydrazone-linked doxorubicin payload can be employed:

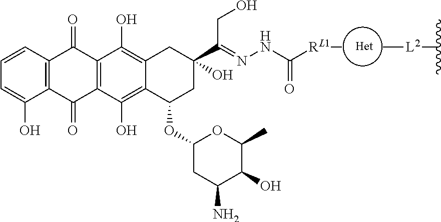

Payloads that include a carboxylic acid functional group can also be linked by a hydrazone:

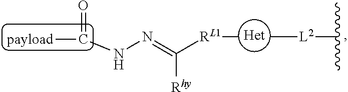

wherein R^{by} represents H or C₁₋₄alkyl, preferably methyl.

By way of example, vincristine type payloads can be conjugated with a hydrazone:

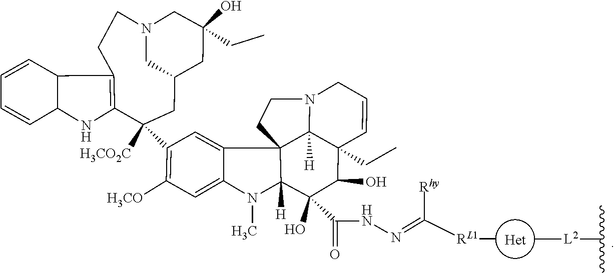

R^{L1} may be represented by the formula:

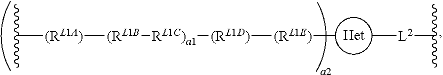

wherein
R^{L1A} is selected from null, C₁₋₁₀alkylene, aryl, —(CH₂CH₂O)ₐ—;
R^{L1B} is selected from null, NHC(O)O, NHC(O)NH, OC(O)NH, O, NR^{zz}, wherein R^{zz} is H or C₁₋₄alkyl, NHC(O), C(O)NH, C₁₋₁₀alkylene, aryl, —(CH₂CH₂O)ₐ—;
R^{L1C} is selected from null, C₁₋₁₀alkylene, aryl, —(CH₂CH₂O)ₐ—;
R^{L1D} is selected from null, NHC(O)O, NHC(O)NH, OC(O)NH, O, NR^{z}, wherein R^{zz} is H or C₁₋₄alkyl, NHC(O), C(O)NH, C₁₋₁₀alkylene, aryl, —(CH₂CH₂O)ₐ—;
R^{L1E} is selected from null NHC(O)O, NHC(O)NH, OC(O)NH, O, NR^{zz}, wherein R^{zz} is H or C₁₋₄alkyl, NHC(O), C(O)NH, C₁₋₁₀alkylene; aryl, —(CH₂CH₂O)ₐ—;
wherein in each case a is independently selected from 1, 2, 3, 4, 5, 6, 7, 8, 9, or 10. In other embodiments, a can be from 1-50, 10-100, 5-10, 5-25, 25-50, or 50-100;
a1 is selected from 1, 2, 3, or 4; and
a2 is selected from 1, 2, 3, 4, 5, 6, 7, or 8.

In some instances, R^{L}1 can have the formula:

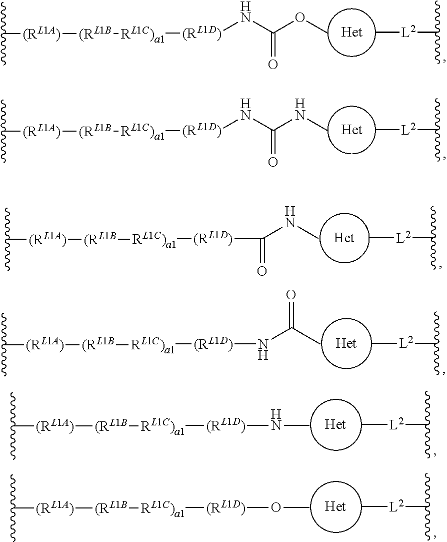

wherein R^{L1A}, R^{L1B}, R^{L1C}, R^{L1D} and a1 are as defined above.

In some instances, R^{L1} can have the formula:

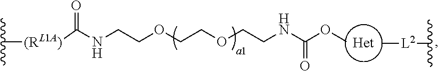

wherein R^{L1A} and a1 are as defined above.

### Methods of Making

The glycoconjugates disclosed herein may be prepared by performing a cycloaddition reaction between a glycoconjugate precursor having the formula:

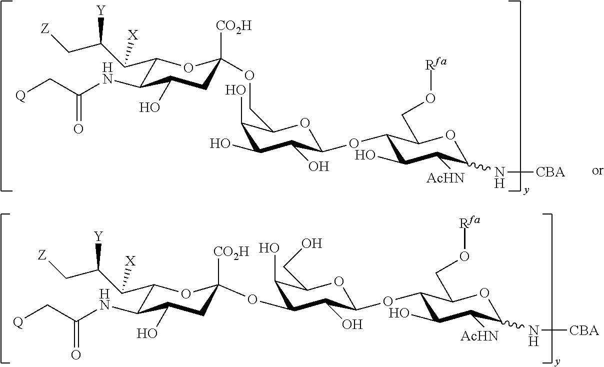

wherein R^{fa}, y, and CBA are as defined above;
Q is H or L2-A^{q};
Z is OH or L²-A^{Z};
Y is OH or L²-A^{y}; and
X is OH or L²-A^{x}; wherein
   L² is as defined above, and A^{q}, A^{x}, A^{Y}, and A^{Z} are each clickable group, with the proviso that at least one of A^{q}, A^{x}, A^{Y}, is A^{Z} present,
   with a payload precursor having the formula:

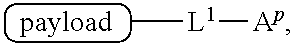

wherein payload and L¹ are as defined above, and A^{p} is complementary clickable group to one of A^{q}, A^{x}, A^{y}, and A^{z} in the glycoconjugate precursor.

“Clickable groups,” as used herein, refer to functional groups that will undergo a cycloaddition reaction under conditions compatible with the cell binding agent, i.e., will not denature or otherwise break apart the polymer chain. Complementary refers to the relationship between the clickable groups in the glycoconjugate precursor and payload precursor. A pair of complementary clickable groups will include a strained cyclic system and a 1,3 dipole or tetrazine. The strained cyclic system includes cyclooctynes and trans-cyclooctenes having the formula:

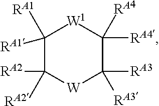

wherein W¹ is either an alkyne or a trans-double bond, e.g.,

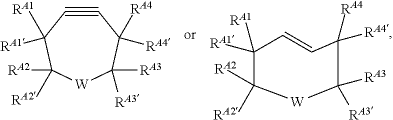

wherein R^{A1}, R^{A1′}, R^{A2}, R^{A2′}, R^{A3}, R^{A3′}, R^{A4}, and R^{A4′} are independently selected from null, H, F, Cl, Br, I, C₁₋₄alkyl, C₁₋₄alkoxy, aryl; and wherein any one of R^{A1}, R^{A1′}, R^{A2}, R^{A2′}, R^{A3}, R^{A3′}, R^{A4}, and R^{A4′} can be L¹ or L², with the proviso that when one of R^{A1}, R^{A1′}, R^{A2}, R^{A2′}, R^{A3}, R^{A3′}, R^{A4}, and R^{A4′} is L¹ or L², the remaining R^{A1}, R^{A1′}, R^{A2}, R^{A2′}, R^{A3}, R^{A3′}, R^{A4}, and R^{A4′} are not L¹ or L²; wherein any two or more of R^{A1}, R^{A1′}, R^{A2}, R^{A2′}, R^{A3}, R^{A3′}, R^{A4}, and R^{A4′} can together form a ring; for instance R^{A1} and R^{A2}, as well as R^{A3} and R^{A4} and can each together form an aromatic ring, while R^{A1′}, R^{A2′}, R^{A3′}, and R^{A4′} are each null;

When one of R^{A1}, R^{A1′}, R^{A2}, R^{A2′}, R^{A3}, R^{A3′}, R^{A4} and R^{A4′} is L¹ or L², W can be CH₂CH₂. When none of R^{A1}, R^{A1′}, R^{A2}, R^{A2′}, R^{A3}, R^{A3′}, R^{A4}, and R^{A4′} are L¹ or L², W can be a group having the formula:

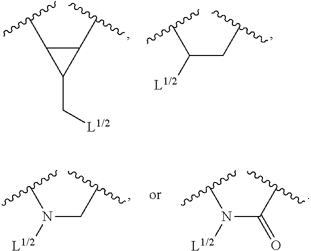

wherein L^{1/2} represents either L¹ or L².

In some cases the strained cyclic system has the formula:

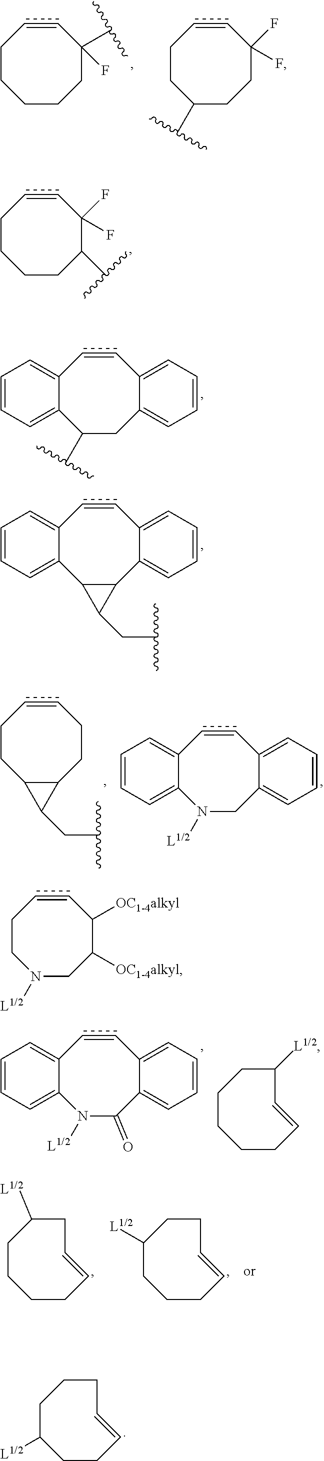

In some cases the strained cyclic system is a trans-cyclooctene having the formula:

Suitable 1,3 dipoles and tetrazines include:

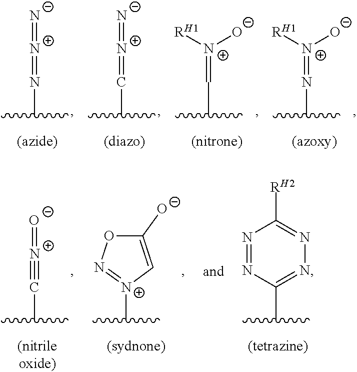

wherein R^{H1} and R^{H2} are as defined above.

In some embodiments, when the glycoconjugate precursor includes a tetrazine, the payload precursor includes a trans-cyclooctene, and vice versa, i.e., the glycoconjugate precursor includes a trans-cyclooctene, the payload precursor includes a tetrazine. When the glycoconjugate precursor includes an azide, diazo, nitrone, azoxy, nitrile oxide, or sydnone, the payload precursor can include cyclooctyne, and vice versa. By taking advantage of the differing reactivities between different pairs of A^{q}, A^{x}, A^{Y}, A^{z}, and AP groups, a single sialoside reside can be conjugated to multiple payloads with a high degree of control.

The reaction between the glycoconjugate precursor and payload precursor is preferably performed in an aqueous buffer solution, such as for example phosphate, buffered saline (e.g. phosphate-buffered saline, tris-buffered saline), citrate, HEPES, tris and glycine. Preferably, the buffer solution is phosphate-buffered saline (PBS) or tris buffer.

The reaction may be carried out at a temperature between about 4 to about 50° C., more preferably between about 10 to about 45° C., even more preferably between about 20 to about 40° C., and most preferably in the range of about 30 to about 37° C. The reaction may be carried out at a pH in the range of about 5 to about 9, preferably in the range of about 5.5 to about 8.5, more preferably in the range of about 6 to about 8. Most preferably, the reaction is carried out at a pH in the range of about 7 to about 8.

In certain cases, the first compound can include mixtures of the 2,6 and 2,3 linked glycoconjugate precursors depicted above. In other embodiments, the glycoconjugate precursor can be substantially only the 2,6 linked oligosaccharide, or substantially on the 2,3 linked oligosaccharide. In some embodiments, the glycoconjugate precursor can be at least 90%, at least 95%, at least 98%, or at least 99% of the 2,6 linked oligosaccharide, while in other embodiments, the glycoconjugate precursor can be at least 90%, at least 95%, at least 98%, or at least 99% of the 2,3 linked oligosaccharide.

In preferred embodiments, the oligosaccharide in the glycoconjugate precursor can be bound to the CBA with an β-N-glycosidic linkage:

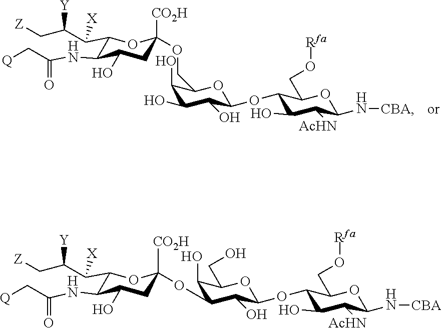

In certain embodiments, the cell-binding agent is an antibody, and the oligosaccharide is conjugated to the antibody through an asparagine side chain via an β-N-glycosidic bond:

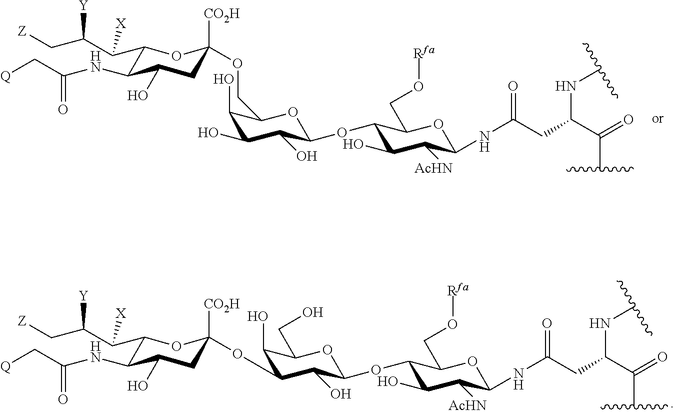

In some cases the GlcNAc moiety is conjugated to the antibody at the asparagine 297 (Asn297) residue according to the EU index as set forth in Kabat. In certain embodiments wherein y is 2, the GlcNAc moiety can be conjugated to both Asn297 residues in the Fc domain. In embodiments wherein y is 1, the GlcNAc moiety can be conjugated to one of the Asn297 residues in the Fc domain. When the antibody has been modified, either by chain elongation or truncation, the oligosaccharide can be conjugated to the asparagine residue corresponding to Asn297 in the unmodified antibody.

In certain embodiments, the glycoconjugate precursor is characterized by Q=L2-A^{q}, and each of X, Y, Z are OH. In certain embodiments, the glycoconjugate precursor is characterized by Q=H, Z=L²-A^{z}, and X and Y are both OH. In yet further embodiments, the glycoconjugate precursor is characterized by Q=L²-A^{q}, Z=L²-A^{z}, and X and Y are both OH. In such cases, A^{q} and A^{Z} may be the same or different. For instance, A^{q} can include a tetrazine, while A^{Z} can include a 1,3 dipole, for instance an azide, and vice versa. In other instances, A^{q} can include a trans-cyclooctene, while A^{Z} can include a cyclooctyne.

The glycoconjugate precursors may be obtained by glycosylating a disaccharide acceptor having the formula:

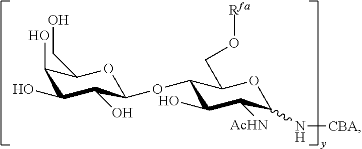

wherein R^{fa}, y, and CBA are as defined above, with a sialoside donor having the formula:

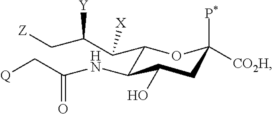

wherein Q, Z, Y, and X are as defined above, and P* is a nucleotide, for instance uridine phosphate, guanosine phosphate, or and cytidine phosphate. Unless specified to the contrary, the term “phosphate” includes any number of sequential phospho-ester bonds, e.g., monophosphate, diphosphate, triphosphate, etc. In preferred embodiments, the sialoside donor has the formula:

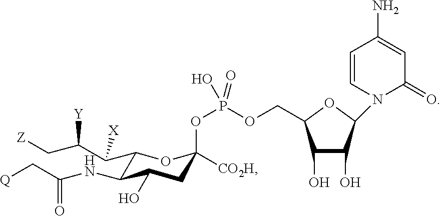

The disaccharide acceptor and sialoside donor can be contacted by at least one sialyltransferase under conditions and for a time sufficient to form the glycoconjugate precursor. The sialyltransferase may be derived from mammals, fishes, amphibians, birds, invertebrates, or bacteria. In one embodiment, the sialyltransferase is an α-(2,3)-sialyltransferase. In another embodiment, the sialyltransferase is an α-(2,6)-sialyltransferase. In yet another embodiment, the sialyltransferase is an α-(2,8)-sialyltransferase. In a preferred embodiment, the sialyltransferase is an α-(2,6)-sialyltransferase, preferably a β-galactoside α-(2,6)-sialyltransferase 1 (ST6Gal 1). In a preferred embodiment, the sialyltransferase is a mammalian sialyltransferase. In other embodiments, the sialyltransferase rat β-galactoside α-2,6-sialyltransferase 1 (ST6Gal 1); Pasteurella multocida α-(2,3)-sialyltransferase; or CMP-N-acetylneuraminate-β-galactosamide-α-2,3-sialyltransferase (ST3Gal IV).

The glycosylation with the sialoside donor may be carried out in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer. The glycosylation is preferably performed at a temperature in the range of about 4 to about 50° C., more preferably in the range of about 10 to about 45° C., even more preferably in the range of about 20 to about 40° C., and most preferably in the range of about 30 to about 37° C. The glycosylation can be carried out at a pH in the range of about 5 to about 9, preferably in the range of about 5.5 to about 8.5, more preferably in the range of about 6 to about 8. Most preferably, the glycosylation is performed at a pH in the range of about 7 to about 8.

In some embodiments, the disaccharide acceptor can be connected to the CBA through a β-N-glycosidic bond:

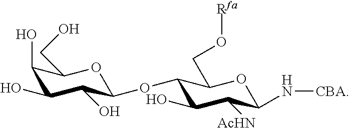

In certain embodiments, the cell-binding agent is an antibody, and the disaccharide acceptor is conjugated to the antibody through an asparagine side chain via an β-N-glycosidic bond:

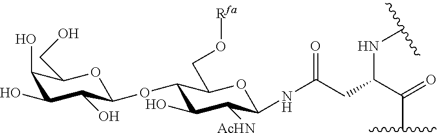

In some cases the GlcNAc moiety is conjugated to the antibody at the asparagine 297 (Asn297) residue according to the EU index as set forth in Kabat. In certain embodiments wherein y is 2, the GlcNAc moiety can be conjugated to both Asn297 residues in the Fc domain. In embodiments wherein y is 1, the GlcNAc moiety can be conjugated to one of the Asn297 residues in the Fc domain. When the antibody has been modified, either by chain elongation or truncation, the oligosaccharide can be conjugated to the asparagine residue corresponding to Asn297 in the unmodified antibody.

The disaccharide acceptor may be prepared by a process including the step of remodeling of an antibody to give a truncated N-glycan acceptor having the formula:

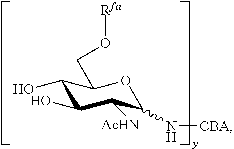

wherein R^{fa}, y, and CBA are as defined above, and a step of glycosylating the truncated N-glycan acceptor with a galactose donor.

In some embodiments, the truncated N-glycan acceptor can be connected to the CBA through an β-N-glycosidic bond:

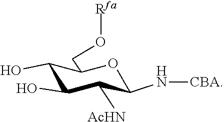

In certain embodiments, the cell-binding agent is an antibody, and the GlcNAc residue (with or without C-6 fucose) is conjugated to the antibody through an asparagine side chain via an β-N-glycosidic bond:

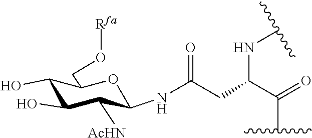

In some preferred cases, the GlcNAc acceptor is characterized when R^{fa} is fucose, preferably L-fucose, and even more preferably α-L-fucose.

In some cases the GlcNAc residue is conjugated to the antibody at the asparagine 297 (Asn297) residue according to the EU index as set forth in Kabat. In certain embodiments wherein y is 2, the GlcNAc residue can be conjugated to both Asn297 residues in the Fc domain. In embodiments wherein y is 1, the GlcNAc residue can be conjugated to one of the Asn297 residues in the Fc domain. When the antibody has been modified, either by chain elongation or truncation, the oligosaccharide can be conjugated to the asparagine residue corresponding to Asn297 in the unmodified antibody.

The truncated N-glycan acceptor can be obtained by remodeling any suitable antibody as disclosed herein. In some embodiments, the remodeling is performed using an endoglycosidase, for instance endoglycosidases classified into EC3.2.1.96. In some embodiments, the endoglycosidase includes endo-β-N-acetylglucosaminidase D (endoglycosidase D, Endo-D, or endo-D), endo-β-N-acetylglucosaminidase H (endoglycosidase H, Endo-H, or endo-H), endoglycosidase S (EndoS, Endo-S, or endo-S), endo-β-N-acetylglucosaminidase M (endoglycosidase M, Endo-M, or endo-M), endo-β-N-acetylglucosaminidase LL (endoglycosidase LL, EndoLL, Endo-LL, or endo-LL), endo-β-N-acetylglucosaminidase F1 (endoglycosidase F1, Endo-F1, or endo-F1), endo-β-N-acetylglucosaminidase F2 (endoglycosidase F2, Endo-F2, or endo-F2), and endo-β-N-acetylglucosaminidase F3 (endoglycosidase F3, Endo-F3, or endo-F3).

In some embodiments a combination of two or more types of endoglycosidases can be used in the remodeling step. For example, several endoglycosidases can be a combination of endoglycosidases having different substrate specificity that are classified into EC3.2.1.96. Exemplary combinations include endoglycosidase D and endoglycosidase S; endoglycosidase S and endoglycosidase LL; endoglycosidase D and endoglycosidase LL; endoglycosidase D and endoglycosidase H; endoglycosidase S and endoglycosidase H; endoglycosidase F1 and endoglycosidase F2; endoglycosidase F1 and endoglycosidase F3; endoglycosidase F2 and endoglycosidase F3; endoglycosidase D, endoglycosidase S and endoglycosidase LL; endoglycosidase D, endoglycosidase S and endoglycosidase H, and endoglycosidase D, endoglycosidase S and endoglycosidase F1.

The remodeling may be carried out in a suitable buffer solution, such as for example phosphate, buffered saline (e.g. phosphate-buffered saline, tris-buffered saline), citrate, HEPES, tris and glycine. Suitable buffers are known in the art. Preferably, the buffer solution is phosphate-buffered saline (PBS) or tris buffer. The remodeling is preferably performed at a temperature in the range of about 4 to about 50° C., more preferably in the range of about 10 to about 45° C., even more preferably in the range of about 20 to about 40° C., and most preferably in the range of about 30 to about 37° C. The remodeling can be carried out at a pH in the range of about 5 to about 9, preferably in the range of about 5.5 to about 8.5, more preferably in the range of about 6 to about 8. Most preferably, the process is performed at a pH in the range of about 7 to about 8.

In some embodiments, the antibody can be remodeled such that a GlcNAc residue (with or without C-6 fucose) is linked to Asn297 (either on one or both heavy chains), and no other glycan structures are present on the antibody.

The truncated N-glycan acceptor may be purified according to conventional techniques, or may be used directly in the galactosylation. The disaccharide acceptor as described above may be prepared by combining the truncated N-glycan acceptor, a galactosyl donor, and a galactosyl transferase. Suitable galactosyl donors include galactosyl nucleotides, including UDP-galactose.

The glycoconjugates disclosed herein can be provided to patients in need thereof in pharmaceutical compositions that contain the glycoconjugate and a pharmaceutically acceptable carrier. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier).

The compositions can additional pharmaceutically acceptable excipients. For instance, buffering agents, including, for example, acetic acid in a salt, citric acid in a salt, boric acid in a salt, and phosphoric acid in a salt, preservatives, such as benzalkonium chloride, chlorobutanol, parabens, and thimerosal.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, Pa.

Preparation of pharmaceutical compositions of the invention and their various routes of administration can be carried out in accordance with methods well known in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Generally the glycoconjugates disclosed herein are provided in a suitable package, e.g., in a vial, pouch, ampoule, and/or any container appropriate for a therapeutic or detection method. Kit components can be provided as concentrates (including lyophilized compositions), which may be further diluted prior to use, or they can be provided at the concentration of use.

### EXAMPLES

The following examples are for the purpose of illustration of the invention only and are not intended to limit the scope of the present invention in any manner whatsoever.

### Reagents and Materials

All chemicals were obtained from Sigma, unless otherwise noted. Cytidine-5′-(5-acetamido-9-azido-3,5,9-tri-deoxy-β-D-glycero-D-galacto-2-nonulopyranosylonic acid monophosphate) (CMP-Neu5Ac9N3) and recombinant rat α-(2,6)-sialyltransferase (GFP-ST6Gal I) were prepared according to the report previously (http://dx.doi.org/10.1002/ante.201307095).

Endo-BCN-PEG4-acid can be prepared according to the procedure described in WO 2016/053107 at page 142, and is also available from a number of commercial suppliers.

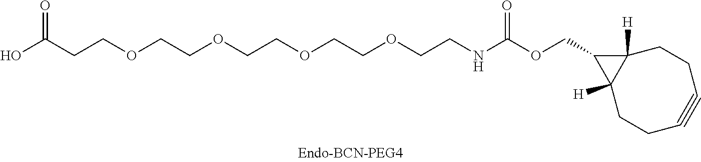 [SMILES: O=C(O)CCOCCOCCOCCOCCNC(=O)OC[C@@H]1[C@@H]2CCC#CCC[C@@H]21]

### Example 1: Synthesis of Clickable Payload

A cytotoxic warhead containing a single primary amine group (designated herein WH—NH₂) was conjugated to Endo-BCN-PEG4 as follows.

WH—NH₂ (600 mg, 1.0 eq), Endo-BCN-PGE4-acid (1.2 eq) and EDCl-HCl (1.2 eq) were taken up in DCM (15 vol, 2% MeOH) and stirred at 0-5° C. Upon completion of reaction, the reaction was quenched with purified water (10 vol). The aqueous layer was separated, and the organic layer was washed with brine, dried over sodium sulphate and concentrated under reduced pressure to give crude WH—NH-Endo-BCN-PEG4 (650 mg, 93.2%, 73.57% HPLC purity).

Crude WH—NH-Endo-BCN-PEG4 (400 mg) was purified by RP-HPLC (C18, MeCN:H₂O) and product containing fractions were combined and lyophilized to give WH—NH-Endo-BCN-PEG4 as a white solid (130 mg, 33%, 94.61% HPLC purity).

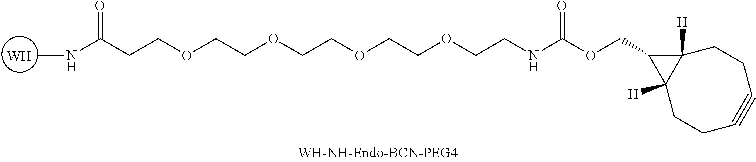

### Example 2: Synthesis of CMP-Neu5N₃ and CMP-Neu9N₃

Sialic acid aldolase (0.2 U/μL, 5 μL), and CMP-sialic acid synthetase (0.2 U/μL, 5 μL) were added to a mixture of N-azidoacetyl-D-mannosamine (5 mg, 0.019 mmol) in tris-HCl buffer (100 mM, pH 8.9, 20 mM MgCl2, 1.9 mL), containing sodium pyruvate (10.5 mg, 0.095 mmol) and CTP (10 mg, 0.019 mmol). The tube was incubated at 37° C., and progress of the reaction was monitored by TLC (EtOH:aq. NH₄HCO₃ (1 M) 7:3, v:v), which after 5 hour indicated completion of the reaction. EtOH (3 mL) was added, and the precipitate was removed by centrifugation and the supernatant was concentrated under reduced pressure. The residue was redissolved in distilled water (500 μL) followed by lyophilization to provide a crude material that was applied to a Biogel fine P-2 column (50*1 cm, eluted with 0.1 M NH₄HCO₃ at 4° C. in dark.). The product was detected by TLC, and appropriate fractions were combined and lyophilized to provide CMP-Neu5N3 as an amorphous white solid (10.1 mg, 81%).

¹H NMR (300 MHz, d₂_{^{O}}) δ 7.82 (d, J=7.5 Hz, 1H, H-6, cyt), 5.97 (d, J=7.6 Hz, 1H, H-5, cyt), 5.84 (d, J=4.2 Hz, 1H, H-1, rib), 4.18 (dd, J=7.4, 4.4 Hz, 2H, H-2+H-3, rib), 4.14-4.04 (m, 4H, H-4+H-5 rib, H-6 Neu), 4.04-3.97 (m, 1H, H-4), 3.95 (s, 2H, N₃CH₂CO), 3.87 (t, J=10.2 Hz, 1H, H-5), 3.82-3.74 (m, 1H, H-8), 3.72 (m, 1H, H-9a), 3.49 (d, J=11.8 Hz, 1H, H-9b), 3.30 (d, J=9.5 Hz, 1H, H-7), 2.36 (dd, J=13.3, 4.6 Hz, 1H, H-3 eq), 1.51 (td, J=12.0, 5.6 Hz, 1H, H-3ax). HRMS (ESI): m/z calcd for C₂₀H₃₀N₇O₁₆P [M−H]⁻: 654.1414; found: 653.9477.

CMP-Neu9N3 was prepared following the reported procedure. CTP (126 mg, 0.24 mmol) was added to a solution of 5-Acetamido-9-azido-3,5,9-tri-deoxy-D-glycero-D-galacto-2-nonulosonic acid (50 mg, 0.15 mmol) in a Tris-HCl buffer (0.1 M, 9 mL, pH 8.9) containing MgCl2 (20 mM). The recombinant CMP-sialic acid synthetase from N. meningitis (4.0 U) and the inorganic pyrophosphatase from S. cerevisiae (2.0 U) were added and the reaction mixture was incubated at 37° C. with shaking. The progress of the reaction was monitored by TLC (isopropanol: 20 mM NH₄OH, 4:1, v:v), which after 3 h indicated completion of the reaction. Ethanol (80 mL) was added and the mixture was kept on ice for 2 h prior to centrifugation. The supernatant was decanted and the pellet (mostly inorganic salts) was re-suspended in EtOH (30 mL), cooled on ice for 1 h and centrifuged. The combined ethanol extracts were concentrated in vacuo providing crude material (168 mg). Ethanol (1.8 mL) was slowly added to the material dissolved in H₂O (0.2 mL) and precipitation occurred immediately. The mixture was kept on ice for 2 h. Next, the supernatant was removed after centrifugation and the white pellet was dried and purified on a column of extra-fine Biogel P-2 eluted with 0.1 M NH₄HCO₃ at 4° C. The appropriate fractions were detected by UV and TLC (as above), collected, concentrated in vacuo (bath temperature<25° C.) and lyophilized to afford CMP-Neu9N3 (60 mg, 62%). ¹H NMR (D₂O containing 0.1 M NH₄HCO₃, 600 MHz): δ 7.82 (d, 1H, J_{5,6}=7.8 Hz, H-6, cyt), 5.97 (d, 1H, J_{5,6}=7.8 Hz, H-5, cyt), 5.82 (d, 1H, J_{1,2}=4.8 Hz, H-1 rib), 4.17 (t, 1H, J=4.8), 4.13 (t, 1H, J=4.8 Hz), 4.08 (m, 3H,), 3.99 (d, 1H, J=12.0 Hz), 3.90 (m, 2H), 3.78 (t, 1H), 3.49 (dd, 1H, J=2.4, 13.2 Hz, H-9a), 3.35 (dd, 1H, J=6.0, 13.2 Hz, H-9b), 3.31 (dd, 1H, J=9.6 Hz), 2.33 (dd, 1H, J_{3eq,4}=4.8 Hz, J_{3eq,3ax}=13.2 Hz, H-3eq), 1.90 (s, 3H, Me), 1.55 (ddd, 1H, J_{3ax,P}=6.0 Hz, J_{3ax,3eq}=13.2 Hz, J_{3ax,4}=12.0 Hz, H-3ax); ¹³C NMR (D₂O, containing 0.1 M NH₄HCO₃, 600 MHz): δ 174.2, 170.4, 166.0, 160.7, 141.4, 96.5, 88.8, 82.9, 74.2, 71.5, 69.3, 69.1, 67.4, 64.9, 53.0, 51.7, 41.0, 22.0; ESI-MS: calcd for C₂₀H₂₈N₇O₁₅P²⁻ [M+H]⁻: m/z: 638.1470; found 638.1421.

### Example 3: Glycan Remodeling

Analysis of Glycopeptides from Tryptic Digestion of Antibodies

An aliquot of an IgG antibody was dried by Speed Vac (Savant SC 110) and re-dissolved in an ammonium bicarbonate buffer (50 mM, pH 8.4) and heated at 100° C. for 5 min to denature the glycoprotein. After cooling the mixture to RT, trypsin (trypsin/IgG=1/30, w/w) was added and the solution was incubated at 37° C. for 22 h, after which it was heated to 100° C. for 5 min to deactivate trypsin. The solution was passed through a C18 reversed phase cartridge, washed with 5% aqueous acetic acid and eluted with a gradient of 2-propanol/5% acetic acid (20-100%) to give glycopeptides which were subjected to LCMS-IT-TOF Mass Spectrometer (Shimadzu) equipped with a XBridge-BEH amide-HILIC columns (Waters, Milford, MA, USA; 2.1×150 mm, 3.5 μm particle size). These separations were carried out at a flow rate of 0.16 ml/min at 20° C., with a mobile phase A consisting of 100 mM ammonium formate in water (adjusted to pH 3.4-3.6 with formic acid) and mobile phase B as pure ACN.

### Native Intact IgG Analysis

Prior analysis, samples were desalted using ZEBA spin columns, diluted to 2 mg/ml, injection 0.5 uL (=1 ug). Samples were run/injected with an Agilent 1290 Infinity LC equipped with on an Acquity 300 Å C18 2.1×50 mm over a gradient of 20-40% B (=70% IPA/20% ACN/10% H₂O/0.1% FA) vs A (0.1% FA in H₂O) at 70° C. in 10 minutes. The detector was an Agilent 6560 Ion Mobility Q-TOF LC/MS, where for this purpose only the Q-TOF functionality was used. Post run analysis and deconvolution was performed using Agilent's Bioconfirm software.

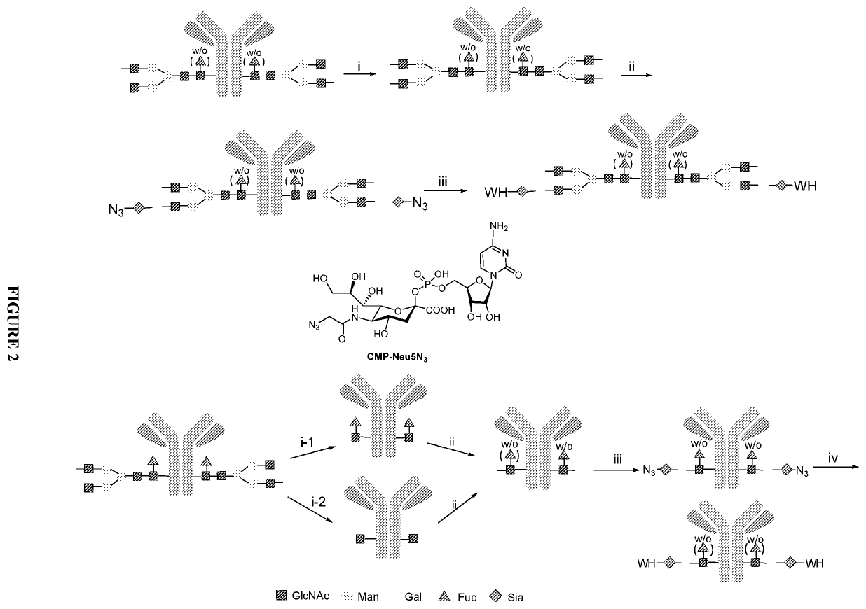

### General Procedure for Modification of IgGs Using App2 (FIG. 2)

### Endo S. Treatment.

Trimming of IgG glycan was undergone using Endo S cloned from Streptococcus pyogenes and overexpressed as a fusion to the chitin binding domain in E. coli. (New England BioLabs). To the IgG antibody (10 mg/mL) in 30 mM histidine, 200 mM sorbitol and 0.02% tween-20, Endo S (0.13 mL, 100 kU/mL) in 10 mM Tris, 25 mM NaCl, 2.5 mM EDTA, 2.5 mM CaCl2), 25 mM sodium acetate was added. The resulting solution was incubated for approximately 48 hours at 37° C. followed by Protein A Sepharose Column (GE Healthcare) purification, buffer exchanging and concentrated into 1.2 mL of 50 mM MOPS containing 20 mM MnCl₂.

### Galactosylation of the IgG

Galactosylation of IgG bearing truncated N-glycan was achieved by addition of β-1,4-galactosyl transferase (200 μg/mL) to the Endo S treatment resulting material in 50 mM MOPS, 20 mM MnCl2, 10 mM UDP-galactose, pH 7.2, 80 μg/mL BSA, 85 U/mL calf intestine alkaline phosphatase and incubation at 37° C. for 70 h. To ensure complete galactosylation, an additional aliquot of UDP-galactose and galactosyl transferase were added to the reaction and incubated at 37° C. for an additional 24 h. The galactosylated IgG was purified using a Protein A Sepharose Column and the solution was exchanged in 50 mM cacodylate, pH 7.6 using an Amicon 10 kDa cutoff spin concentrator (Millipore).

### Sialylation of the IgG

The sialylation of galactosylated IgG was performed in 50 mM cacodylate, 14 mg/mL of IgG, 8 mM CMP-Neu5N3, 90 μg/ml BSA, 90 U/mL calf intestine alkaline phosphatase and 0.4 mg/mL GFP-ST6Gal I at pH 7.6 and incubated at 37° C. for 4 days followed by Protein A Sepharose Column purification and buffer exchanging to 50 mM cacodylate. The extent of sialylation was monitored by LC-MS as described previously using a Shimadzu LCMS-IT-TOF Mass Spectrometer. Following every 48 hours incubation, the sample was buffer exchanged with 50 mM cacodylate, pH7.6 using an Amicon 10 kDa cutoff spin concentrator to remove CMP, an inhibitor of ST6Gal I and an additional aliquot of CMP-Neu5N3 and α2-6 sialyltransferase were added back to this washed preparation.

A key discovery was the unexpected ability of wild-type human β4GalT1 galactosyl-transferase to transfer a galactose residue onto a α1-6 fucosylated GlcNAc residue. This reaction does not occur in nature. Moreover, it was found that the resulting disaccharide could be further modified with the addition of an azido-modified sialic acid by ST6Gal1 sialyltransferase.

### Example 4: Conjugation with a Clickable Payload

10 mg/ml of Her2 from Example 3 in 50 mM Cacodylate buffer pH 7.6 was conjugated by the addition of 7.5 molar equivalents of WH—NH-Endo-BCN-PEG4 (Example 1) (10 mM stock in DMA) and DMA to a final cosolvent level of 20% v/v. The conjugation reaction was incubated in a 20° C. water bath overnight, and then purified by PLRP. A DAR by PLRP of 1.8 was achieved.

The resulting glycoconjugate is herein termed ‘Her-App2’.

### Example 5: Preparation of a Biantennary Antibody Drug Conjugate

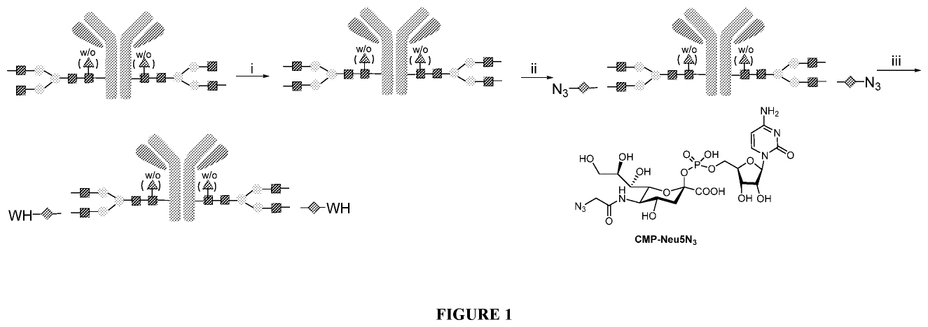

The N-linked oligosaccharides on the Herceptin antibody were remodeled according to the methods described in Li et al. 2014 (Angew. Chem. Int. Ed. Engl., 2014, Jul. 7; 53(28):7179-82). See FIG. 1. 9.2 mg/ml of the remodeled Her2 (Example 3) in 50 mM Cacodylate buffer pH 7.6 was conjugated by the addition of 20 molar equivalents of WH—NH-Endo-BCN-PEG4 (Example 1) (10 mM stock in DMA, structure provided in FIG. 1) and DMA to a final Co solvent level of 20% v/v. The conjugation reaction was incubated in a 20° C. water bath for 66 hours. A DAR by PLRP of 1.4 was achieved.

The resulting glycoconjugate is herein termed ‘Her-App1’.

The activity of the recombinant sialyltransferase ST6Gal1 to the α(1,3)- and α(1,6)-arm of the biantennary N-glycan of the Fc region of antibodies can be differential by controlling the ratio of CMP-sialic acid and antibody. This can result in ADCs having DAR2 or DAR4. However, the careful controlling of the reaction stoichiometry that is required impacts on product reproducibility between batches.

### Example 6: Glycoconjugate Properties

### Physical Properties

Her-App1 and Her-App2 were analyzed by hydrophobic Interaction Chromatography (HIC). This was carried out using column a MabPac HIC-Butyl, 5 μm, 4.6×100 mm column (Thermo, #882558, lot 01425138, serial no. 001303) with a MabPac HIC-Butyl, 5 μm, 4.6×10 mm Guard cartridge: (Thermo, #882559, lot 1425011). With a Mobile Phase A of 1.5 M (NH₄)₂SO₄, 25 mM NaPO₄ (pH 7.4) and a Mobile Phase B of 80% 25 mM NaPO₄ (pH 7.4), 20% CH₃CN. The assay was run at 0.8 ml per minute and a column temperature of 25° C. A 10 μl sample load at 1 mg/ml was used for the analysis.

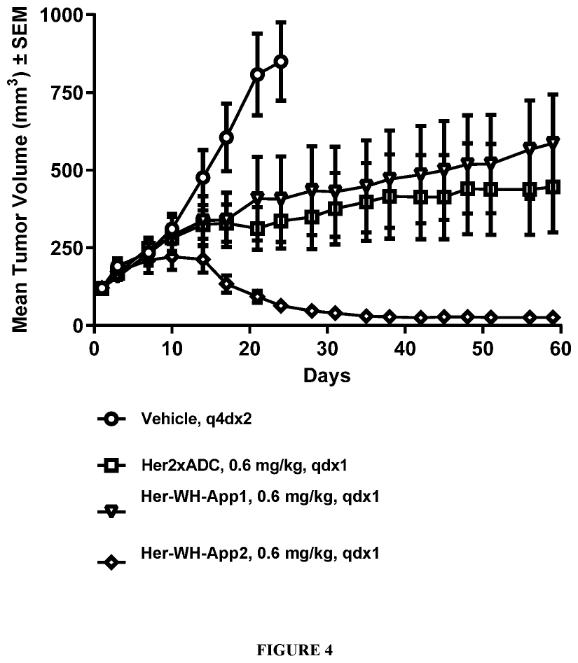

The HIC showed a distinct difference between the two ADCs, with Her-App1 separating into multiple hydrophobic species, whilst Her-App2 eluted as one more hydrophilic peak (see FIG. 4).

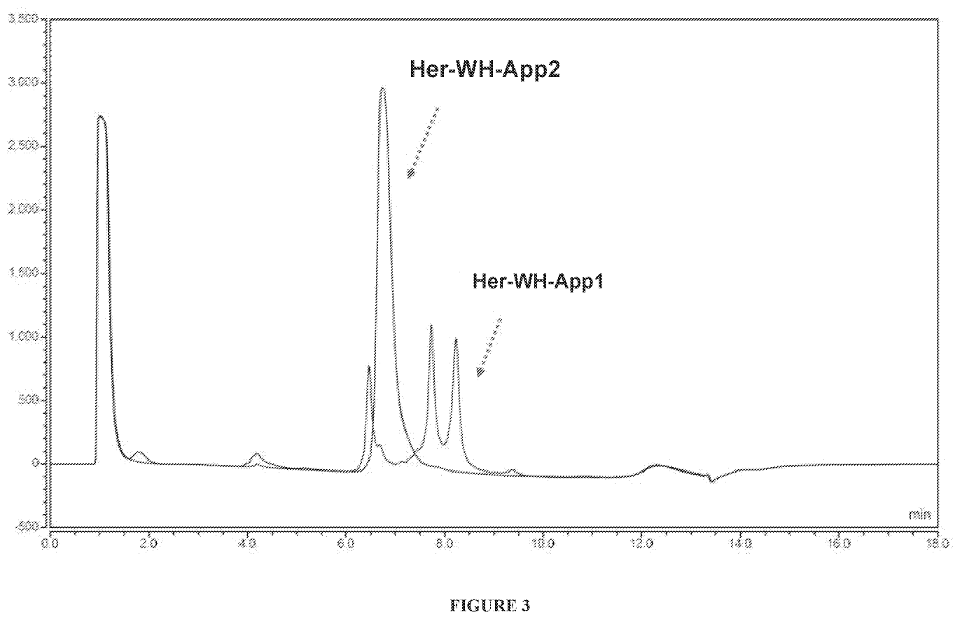

The increased hydrophilicity of Her-App2 relative to Her-App1 is prima facie surprising in view of the fact that Her-App2 has considerably fewer sugar residues than Her-App1 (compare FIGS. 2 and 3). Sugar residues are very hydrophilic moieties, suggesting that the conjugate with a greater number of sugar residues would have higher hydrophilicity. The fact that this is not the case here suggests a more complex interaction is occurring between the antibody, oligosaccharide, and drug-linker elements of the ADC.

### In-Vitro Binding to Her2

Her2 is the cognate antigen of the Herceptin antibody. Binding of Her-App1 and Her-App2 was determined by ELISA. Maxisorp ELISA plates were coated with 0.5 μg/mL recombinant human Her2 at room temperature, before blocking with 3% BSA. Sample titrations were prepared in assay buffer (0.1% BSA/0.05% tween) between 66.6 and 0.016 nM in quartering dilutions. Samples were then incubated on the antigen coated plate for 1 hour. A mouse anti-human antibody conjugated to HRP was used fo detection (Sanquin M1328) and incubated for 1 hour before washing and adding the detection agent, TMB for 10 minutes before stopping the reaction with HCl. Binding absorbance data was acquired on the Spectramax plate reader at 450 nm.

For comparison, Her2 binding was also assessed for ‘Her-C220’ [an unconjugated version of Herceptin in which 3 of the 4 interchain cysteines have been substituted for either V (in the heavy chain) or S (in the light chain)] and B12 [an unconjugated monoclonal antibody against the HIV-1 protein; used here as a control].

| ADC/antibody | IC₅₀ (nM) |
|---|---|
| Her-App1 | 0.23 |
| Her-App2 | 0.38 |
| Her2-C220 | 0.17 |
| B12 | No Binding |

The two ADCs bound to Her2 with similar affinity.

### In-Vitro Cytotoxicity

The in vitro cytotoxicity of Her-App1 and Her-App2 against Her2+ve N87 cells was determined. A “thaw and go” cytotox assay was used to determine the cytoxicity, N87 cells were taken from cryogenic storage and seeded to 5×10⁴ cells/mL (5×10³ cells/well) on an EDGE plate then incubated for a minimum of 2 hours at 37° C./5% CO₂/absolute humidity. An 11 point, 1 in 4 serial titrations of the test and control samples was prepared in duplicate from 500 nM to 0.4768 pM with a final negative control. The titrated samples were added to the EDGE plate containing cells and incubated for 5 days at 37° C./5% CO₂/absolute humidity. Celltiter Aqueous One solution was added and the plate was incubated at 37° C./5% CO₂/absolute humidity for a final time, before measuring absorbance at 490 nm using the SpectraMax plate reader.

For comparison, cytotoxicity was also assessed for ‘Her2xADC’ [Her2-C220 conjugated to tesirine at the C220 residue] and B12-C220-SG3249 [the B12 antibody conjugated to tesirine at the C220 residue].

| ADC | IC₅₀ (pM) |
|---|---|
| Her-WH-App1 | 24.7 |
| Her-WH-App2 | 21.7 |
| Her2xADC | 17.9 |
| B12-C220-SG3249 | 2994 |

Her-App1 and Her-App2 were found to have similar cytotoxicity to each other and also to the benchmark Her2xADC. Significantly less cell kill was observed with the non-Her2-binding B12 control ADC.

### In-Vivo Efficacy

The in vivo efficacy of the Her-App1 and Her-App2 conjugates was measured in the breast cancer Her2+ve BT474 xenograft model. For comparison, in vivo efficacy was also assessed for ‘Her2xADC’.

Female severe combined immunodeficient mice (Fox Chase SCID®, CB17/Icr-Prkdcscid/IcrIcoCrl, Charles River) were ten weeks old with a body weight (BW) range of 16.1 to 21.8 g on Day 1 of the study. On the day of tumor implant, each test mouse received a 1 mm³ BT474 fragment implanted subcutaneously in the right flank, and tumor growth was monitored as the average size approached the target range of 100 to 150 mm³. Tumors were measured in two dimensions using calipers, and volume was calculated using the formula:

Tumor Volume (mm³)=w²×l/2

where w=width and l=length, in mm, of the tumor. Tumor weight may be estimated with the assumption that 1 mg is equivalent to 1 mm³ of tumor volume.

Thirty-six days after tumor implantation, designated as Day 1 of the study, the animals were sorted into groups each consisting of ten mice with individual tumor volumes of 75 to 172 mm³ and group mean tumor volumes of 119-121 mm³. On Day 1 of the study, drugs were administered intravenously (i.v.) in a single injection (qd×1) via tail vein injection. The dosing volume was 0.2 mL per 20 grams of body weight (10 mL/kg), and was scaled to the body weight of each individual animal. Tumors were measured using calipers twice per week, and each animal was euthanized when its tumor reached the endpoint volume of 1000 mm³ or at the end of the study (Day 59), whichever came first. Results are shown in FIG. 4.

The minimal efficacious dose (MED) of Her2xADC and Her-App1 was >0.6 mg/kg, while the MED for Her-App2 was determined to be 0.3 mg/kg.

### Tolerability: Rat Toxicology Study

Her2xADC, Her-App1 and Her-App2 were evaluated in a single intravenous dose rat tolerability study.

Male Sprague Dawley rats (n=3/group) were dosed at 4 mg/kg with Her-App1, 2 mg/kg with Her-App2, or 4 mg/kg with Her-App1 on day 1, with necropsy on day 21 following dosing. Bodyweights and food consumption were monitored frequently with in-life sampling for clinical pathology (blood on days 8 and 21) and repeated sampling for pharmacokinetics. At necropsy, macroscopic observations were taken with selected organs weighed and retained for possible histopathology.

Her2xADC was evaluated at 1.5 mg/kg, single intravenous injection to male Sprague Dawley rats was associated with reduced overall body weight gain (overall bodyweight gain was 39% lower), associated with reduced food consumption. White blood cell numbers were reduced on day 8 (−61%), with evidence of recovery by day 21. At necropsy, reduced thymus, spleen, testes and prostate/seminal vesicle weights and increased adrenal gland weight were observed.

Her-App1 was poorly tolerated at 4 mg/kg, resulting in early euthanasia 11 days after dosing in 2 out of 3 animals. Bodyweight gain was markedly reduced in these animals, with none of the expected weight gain after dosing. Several haematology parameters were markedly reduced on day 8 (reticulocytes (−93%), white blood cells (−86%) and platelets (−66%)), with no evidence of recovery.

Her-App2 was clinically well tolerated at 2 & 4 mg/kg. Bodyweight gain was dose-dependently reduced (overall bodyweight gain was 55% lower at 4 mg/kg), consistent with reduced food consumption. Several haematology parameters were reduced on day 8 (reticulocytes (−52%), white blood cells (−68%) and platelets (−22%)), with evidence of recovery by day 21. At necropsy, dose-dependent reductions in thymus, liver and spleen weights and increased lung weights were noted, with two animals presenting with pale kidneys at 4 mg/kg.

The maximum tolerated dose (MTD) for Her2xADC was 1.5 mg/kg (the highest dose tested).

The maximum tolerated dose (MTD) for Her-App1 was lower than 4 mg/kg.

The maximum tolerated dose (MTD) for Her-App2 was 4 mg/kg.

### Therapeutic Index

The Therapeutic Index (TI) of the ADCs may be calculated by first determining the Human equivalent dose of the MED and MTD and then dividing the HED of the MTD by the HED of the MED, as shown below:

| | mg/kg | | | | |
|---|---|---|---|---|---|
| | MED | MTD Rat | HED of MED | HED of MTD rat | TI |
| Her2xADC | >0.6 | 1.5 | >0.050 | 0.22 | <4 |
| Her-App1 | >0.6 | <4.0 | >0.050 | <0.60 | <<12 |
| Her-App2 | 0.3 | 4.0 | 0.25 | 0.60 | 24 |

MED: Minimal effective dose in mice MTD: Maximal Tolerable dose
HED: Human equivalent dose TI: Therapeutic Index
Her-App2 exhibits a Therapeutic Index of at least twice that of Her-PL1603-App1.
Her-App2 exhibits a Therapeutic Index of about 6 times that of Her2xADC.

### Pharmacokinetics (PK) of Her-App1 and Her-App2 in Rats

Plasma samples of rats dosed with a single dose of 2 and or 4 mg/kg of Her-App1 and Her-App2 and samples were taken 1, 3, 6, 48, 72, 168, 336 and 480 h after dosing. The samples were analysed for total human IgG and WH-conjugated IgG as described in Zammarchi Blood vol 131 (10), 1094-1105 2018.

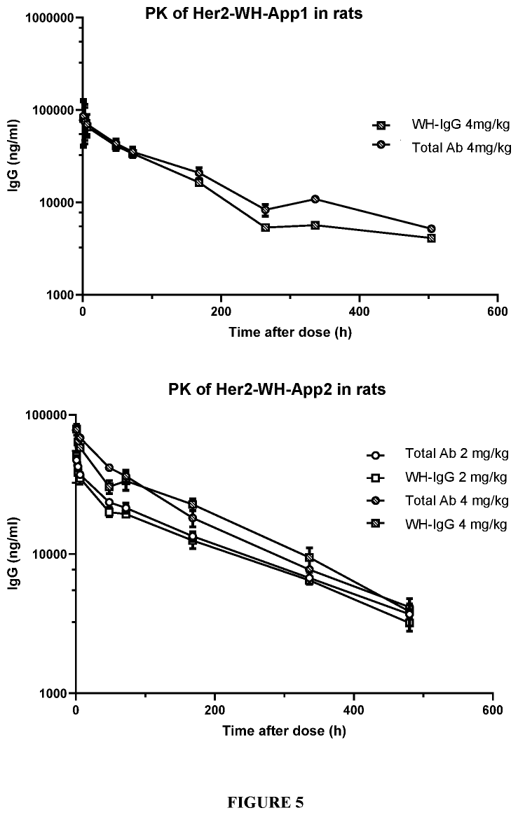

FIG. 5A shows comparable PK profiles of Her2-App1 for total IgG and WH-IgG (i.e., warhead conjugated IgG) at 4 mg, which shows the conjugate is highly stable.

FIG. 5B shows comparable PK profiles of Her2-App2 for total IgG and WH-IgG both at 2 and 4 mg, which shows the conjugate is highly stable.

### Further Comments on Properties

A further advantage of ‘Approach 2’ as described above in Examples 1-4 is that it is easier to control the DAR at 2. In earlier approaches employing an intact glycan, it was more difficult to control the DAR at 2, necessitating careful control of reaction conditions.

In addition, Approach 2 abolishes Fc(gamma) receptor activity which is an advantage for a number of ADC applications.

### Example 7—Additional Conjugations

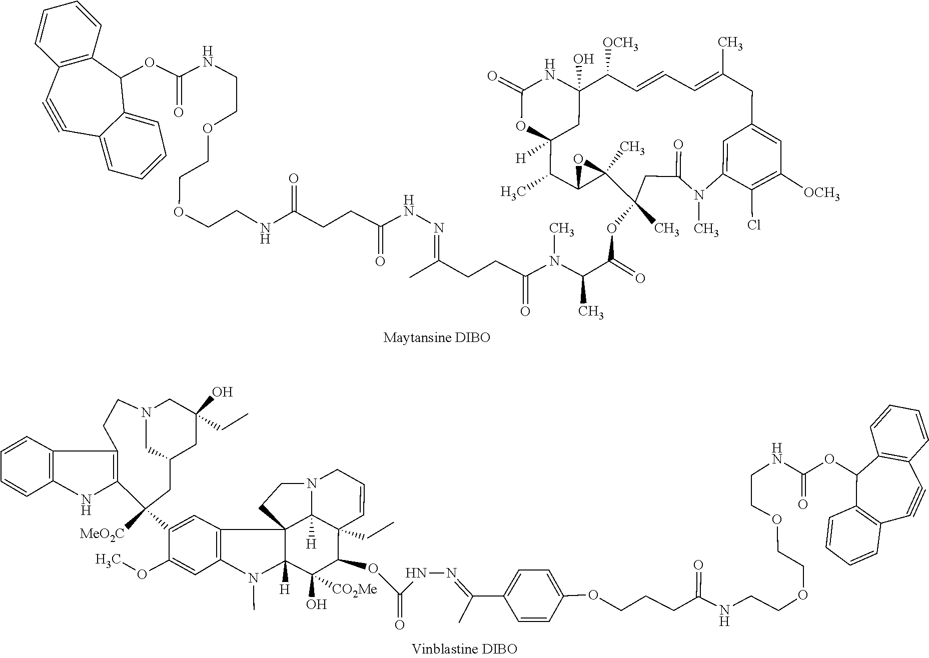

Maytansine DIBO or vinblastine-DIBO in DMF are added to the remodeled antibody of Example 3 in cacodylate buffer, pH 7.6. The mixture is placed in a shaker for 2 h at room temperature and the excess DIBO reagent is removed by washing with cacodylate buffer or PBS buffer in a 7 KDa cutoff Zeba spin columns (Thermo Scientific).

Conjugation

Each of the above Maytansine and vinblastine drug-linkers were conjugated to a Her2 antibody according the general procedure described above in Example 4. In each case, full conversion of the modification was observed by native intact IgG analysis (ie. DAR=2).

### Example 8

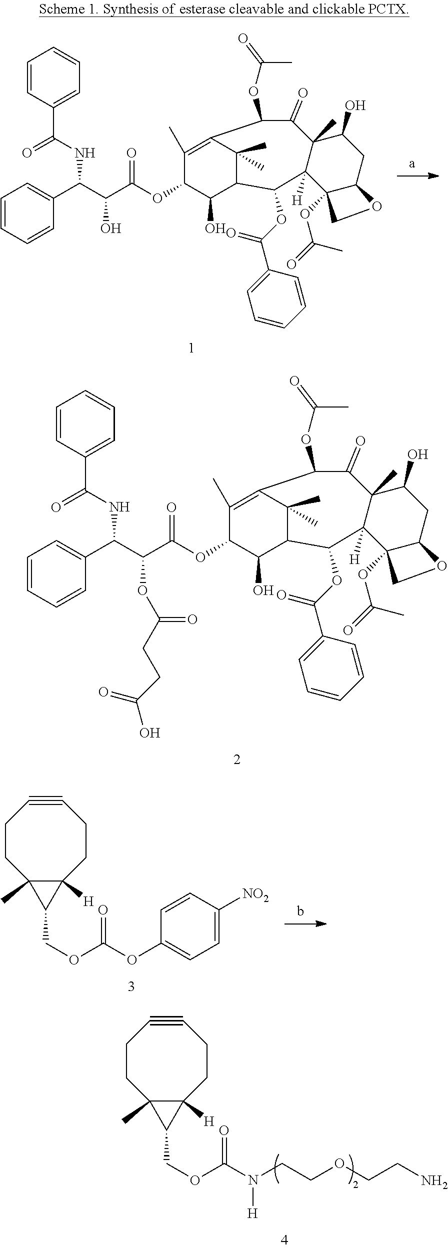

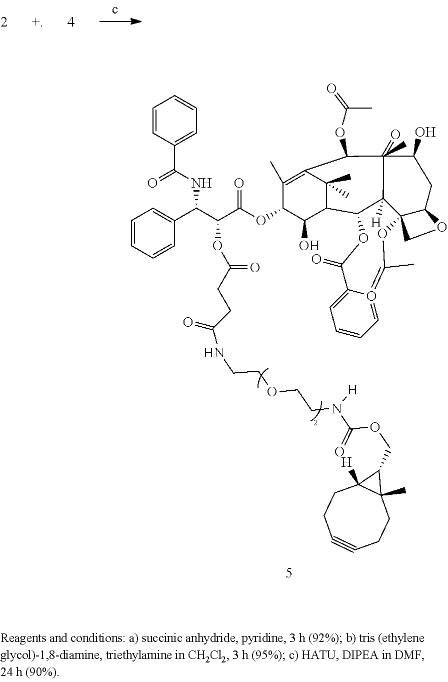

### 4-(((1S,2R)-1-benzamido-3-(((4S,4aS,6R,9S,11S,12S,12aR,12bS)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl)oxy)-4-oxobutanoic acid (8-2)

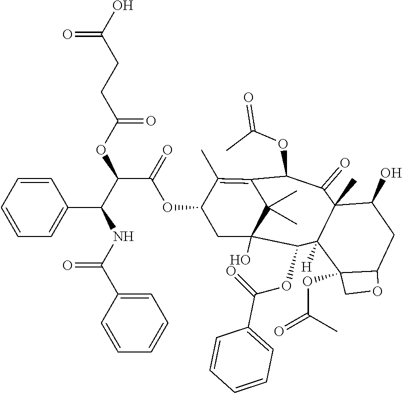

A reaction mixture of 8-1 (0.05 g, 0.06 mmol) and succinic anhydride (0.076 g, 0.76 mmol) in 1.2 mL pyridine was stirred at RT for 3 h. After 3 h, pyridine was evaporated to dryness in vacuo. The residue was then treated with 2 ml of water, stirred for 20 min, and filtered. The obtained precipitate was then dissolved in acetone and water was added slowly, and the fine crystals of product were collected. This yielded 0.048 g (86%) of 8-2.

¹H NMR (DMSO-d6, 500 MHz): δ 12.25 (br s, 1H), 9.19 (d, 1H), 7.94-8.00 (d, 2H), 7.81-7.85 (d, 2H), 7.70-7.73 (m, 1H), 7.63-7.66 (m, 2H), 7.49-7.56 (m, 1H), 7.45-7.50 (m, 2H), 7.40-7.44 (m, 4H), 7.11-7.21 (m, 1H), 6.27 (s, 1H), 5.76-5.83 (t, 1H), 5.73 (s, 1H), 5.50-5.54 (t, 1H), 5.40 (d, 1H), 5.34 (d, 1H), 4.88-4.90 (d, 2H), 4.61 (s, 1H), 4.08-4.11 (m, 1H), 3.97-4.02 (m, 2H), 3.56 (d, 1H), 2.57-2.63 (t, 2H), 2.27-2.37 (m, 1H), 2.22 (s, 3H), 2.09 (s, 3H), 1.76-1.83 (m, 1H), 1.74 (s, 3H), 1.58-1.65 (t, 1H), 1.48 (s, 3H), 1.21 (s, 1H), 0.95-1.00 (d, 6H).

¹³C NMR 134, 131.9, 130, 129.2, 129.20, 129.09, 128.85, 128.09, 127.93, 84.15, 75.76, 75.11, 75, 75.14, 71.41, 71, 55.34, 54.43, 46.51, 40.28, 37.11, 37, 34.86, 34.86, 29.28, 29, 26.74, 23.11, 22.05, 21.16, 14.39, 10.63, 10.33. MALDI HRMS for C₅₁H₅₅NO₁₇ m/z [M+Na⁺] 976.35; found 976.346.

### Bicyclo[6.1.0]non-4-yn-9-ylmethyl (4-nitrophenyl) carbonate (8-3)

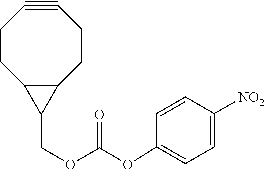 [SMILES: O=C(OCC1C2CCC#CCCC21)Oc1ccc([N+](=O)[O-])cc1]

To a solution of ((1R, 8S, 9r)-Bicyclo [6.1.0] non-4-yn-9-ylmethanol (100 mg, 0.66 mmol) in CH₂Cl₂ (10 mL) was added pyridine (134.70 μL, 1.66 mmol) and 4-nitrophenyl chloroformate (200 mg, 1 mmol). After stirring for 3 h at RT the reaction mixture was quenched with saturated ammonium chloride solution (10 mL) and extracted with CH₂Cl₂ (3×10 mL). The organic layer was dried using MgSO₄ and concentrated in vacuo. The residue was further purified by column chromatography on silica gel (EtOAc:Hexane, 1:5) to afford desired product 8-3 (162 mg, 77%) as a white solid.

¹H NMR (CDCl₃, 500 MHz): δ 8.28 (d, 2H), 7.40 (d, 2H), 4.31 (d, 2H), 2.15-2.5 (m, 6H), 1.35-1.45 (m, 2H), 0.64-0.75 (m, 3H). ¹³C NMR (150 MHz, CDCl3): δ 155.6, 152.5, 145.3, 125.3, 121.7, 98.7, 68.0, 29.0, 21.3, 20.5, 17.2.

### ((1R, 8S, 9r)-Bicyclo [6.1.0] non-4-yn-9-ylmethyl (2-(2-(2-aminoethoxy) ethoxy) ethyl) carbamate (8-4)

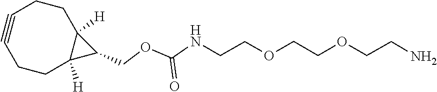 [SMILES: NCCOCCOCCNC(=O)OC[C@H]1[C@@H]2CCC#CCC[C@@H]21]

Et₃N (339 μL, 1.945 mmol) was added to stirred solution of 8-3 (150 mg, 0.389 mmol) and tris (ethylene glycol)-1,8-diamine (569 μL, 3.89 mmol) in CH₂Cl₂ (10 mL). The reaction mixture was stirred for 3 h, after which the solvent was removed under reduced pressure. The residue was purified by flash chromatography over latrobeads (MeOH/CH₂Cl₂, 5 to 25%, v/v) to give compound 8-4 as a light-yellow liquid (116 mg, 92%).

¹H NMR (CDCl₃, 500 MHz): δ 5.48 (br s, NH), 4.15 (d, 2H), 3.5-3.75 (m, 8H), 3.4 (br s, 2H), 2.9 (br s, 2H), 2.5 (br s, 2NH₂), 2.16-2.36 (m, 6H), 1.5-1.65 (m, 2H), 1.2-1.44 (m and s, 3H), 0.79-1.00 (m, 2H)¹³C NMR (150 MHz, CDCl3): δ 98.8, 73.4, 70.3, 70.2, 70.1, 62.7, 41.7, 40.8, 29.1, 21.4, 20.1, 17.8. MALDI HRMS for C₁₇H₂₈N₂O₄ m/z calcd (M+H)⁺ 325.2124, found: 325.2122.

### (4S,4aS,6R,9S,11S,12S,12aR,12bS)-9-(((19R)-19-((S)-benzamido(phenyl)methyl)-1-(bicyclo[6.1.0]non-4-yn-9-yl)-3,14,17-trioxo-2,7,10,18-tetraoxa-4,13-diazaicosan-20-oyl)oxy)-12-(benzoyloxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (8-5)

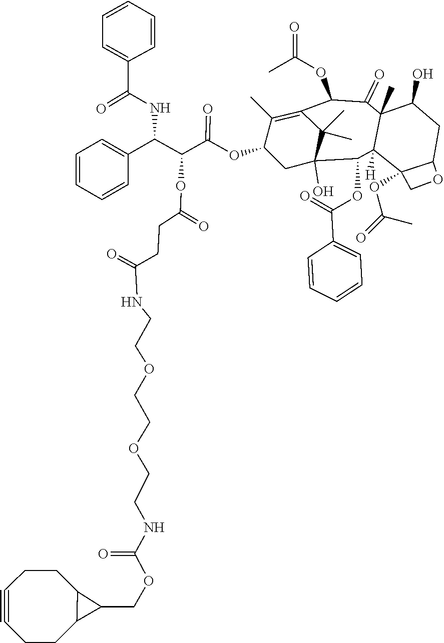

A mixture of 8-2 (5 mg, 0.0052 mmol) and 8-4 (2.1 mg, 0.0062 mmol) was dissolved in anhydrous DMF (1 mL). N, N-diisopropylethylamine (2.73 μL, 0.0157 mmol) and 1-[Bis(dimethylamino) Methylene]-1H-1, 2,3-triazolo [4,5-b] pyridinium 3-oxid hexafluorophosphate (HATU, 3 mg, and 0.00786 mmol) was added sequentially and reaction mixture was stirred for 2 h at RT. TLC showed complete reaction after stirring reaction for 2 h at RT. Solvents were evaporated under reduced pressure, and the crude product was purified by silica gel chromatography using EtOAc:Hexane (5 to 15%, v/v) as a mobile phase giving pure 8-5 as a white solid (6.5 mg, 98%).

¹H NMR (CDCl₃, 500 MHz): δ 8.15 (d, 1H), 7.83 (d, 1H), 7.63 (t, 1H), 7.52 (dt, 2H), 7.48-7.37 (m, 3H), 7.32 (s, 1H), 6.30 (s, 1H), 6.25-6.13 (m, 1H), 5.69 (d, 1H), 5.46 (d, OH), 5.31 (s, OH), 5.03-4.92 (m, OH), 4.32 (d, 1H), 4.21 (d, 1H), 4.13 (q, 1H), 3.96 (d, 1H), 3.81 (d, 1H), 3.60 (d, 4H), 3.48 (s, 1H), 3.37 (s, 2H), 2.77 (t, 1H), 2.54 (d, 2H), 2.44 (s, 1H), 2.39 (d, 1H), 2.35-2.28 (m, 1H), 2.24 (s, 2H), 2.15 (d, 1H), 2.06 (s, 1H), 1.93 (s, 2H), 1.69 (s, 2H), 1.64 (s, 4H), 1.50 (dd, 3H), 1.33-1.20 (m, 5H), 1.14 (s, 2H), 0.93-0.85 (m, 1H), 0.73 (s, 1H).

¹³C NMR (150 MHz, CDCl3): δ 130.22, 127.36, 133.70, 128.72, 131.85, 126.78, 129.00, 128.43, 75.61, 71.74, 53.19, 75.08, 74.37, 84.43, 72.10, 76.42, 76.42, 69.23, 45.60, 70.23, 69.77, 39.36, 40.73, 43.72, 29.44, 35.13, 30.76, 22.68, 33.27, 35.47, 21.39, 21.39, 35.48, 14.82, 35.57, 9.62, 23.39, 9.62, 18.63, 17.32, 33.28, 22.68, 31.61, 29.66, 26.80, 22.13, 14.12, 22.87, 23.69. MALDI HRMS for C₆₈H₈₁N₃O₂₀ m/z calcd (M+Na)⁺ 1282.54, found: 1282.534.

### Conjugation

The above clickable drug-linker (compound 8-5) was conjugated to a Her2 antibody according the general procedure described above in Example 4. Full conversion of the modification was observed by native intact IgG analysis (ie. DAR=2).

### Example 9

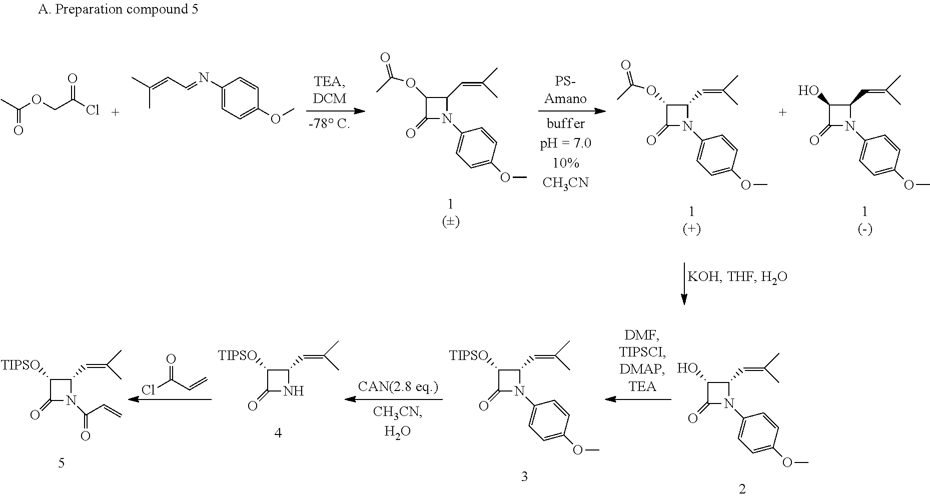

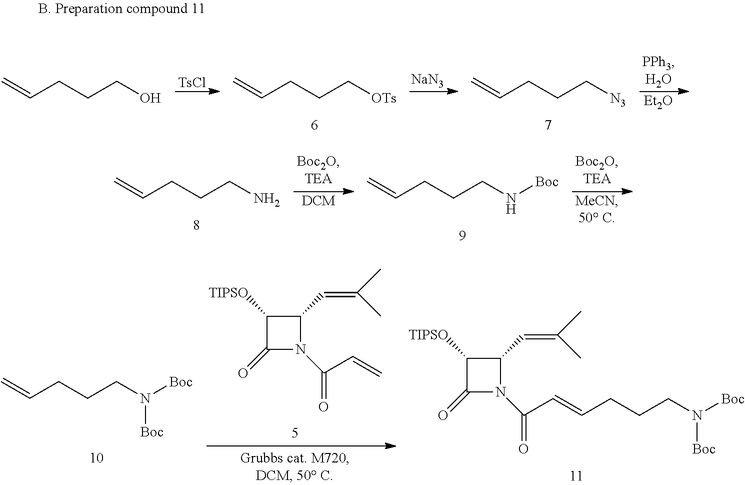

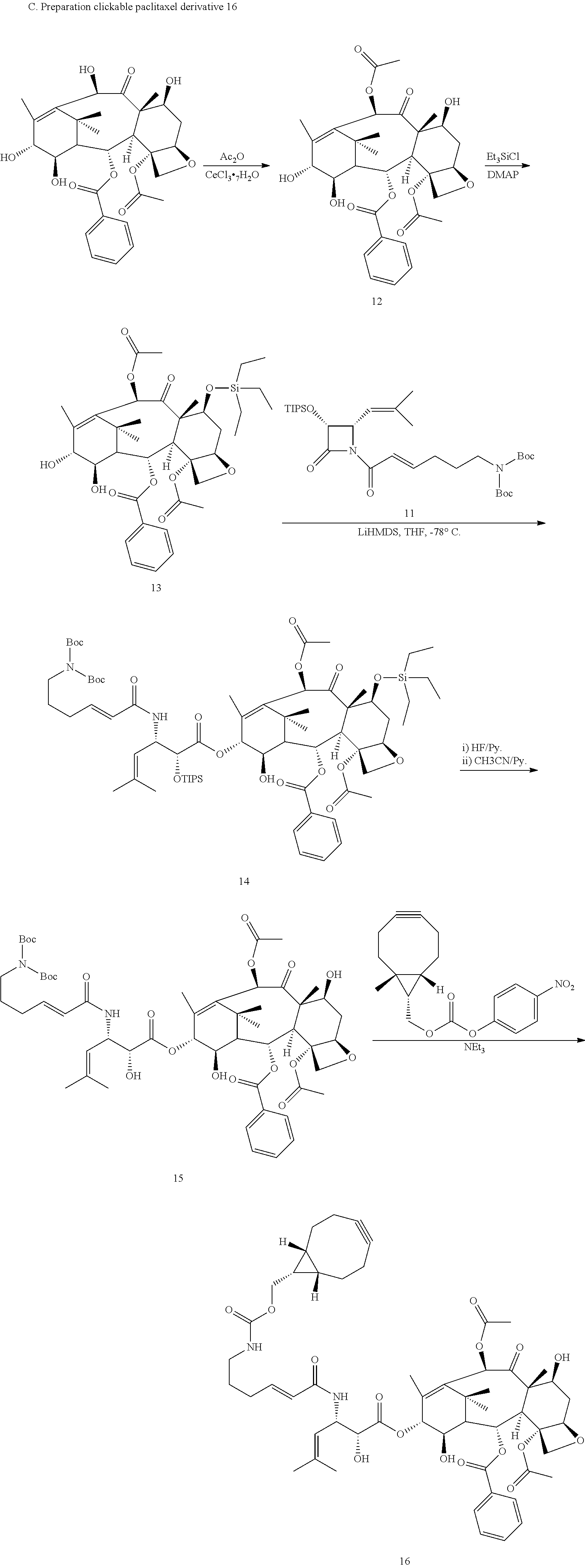

### 1-(4-methoxyphenyl)-2-(2-methylprop-1-en-1-yl)-4-oxoazetidin-3-yl acetate (9-1)

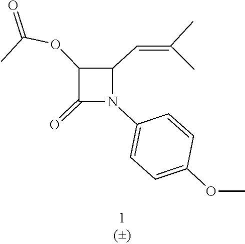 [SMILES: COc1ccc(N2C(=O)C(OC(C)=O)C2C=C(C)C)cc1]

To a solution of 4-methoxyaniline (6.14 g, 50 mmol) in dry CH₂Cl₂ (50 ml) was added 3-methylbut-2-enal (4.62 g, 55 mmol) and 4 Å molecular sieve (5 g) under N2. Then the mixture was stirred at room temperature for 4 h. After fully reacted, filter to remove molecular sieve, and the solvent was removed under vacuum. The residue was dissolved in dry CH₂Cl₂ (50 ml) with TEA (7.58 g 75 mmol) at −78° C. To the solvent was added 2-chloro-2-oxoethyl acetate (8.16 g, 60 mmol) over a 10 min period. Then the reaction was allowed to slowly warm to room temperature. After 12 h, the solvent was washed with saturated NH₄Cl solvent, the water phase was then extracted with CH₂Cl₂ (50 ml*3). The combined organic phase was washed with brine, dried over MgSO₄, concentrated, and purified by silica gel column using EA:Hex (20:1 to 4:1) to give pale yellow solid as yield 69%.

### (2S,3R)-1-(4-methoxyphenyl)-2-(2-methylprop-1-en-1-yl)-4-oxoazetidin-3-yl acetate (9-1(+))

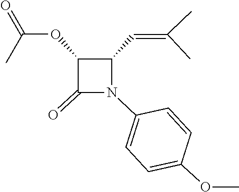 [SMILES: COc1ccc(N2C(=O)[C@H](OC(C)=O)[C@@H]2C=C(C)C)cc1]

To a solution of 1-(4-methoxyphenyl)-2-(2-methylprop-1-en-1-yl)-4-oxoazetidin-3-yl acetate (9-1) (14.50 g, 50 mmol) in 180 ml 0.2 M sodium phosphate buffer (pH=7.4) mixed with 20 ml acetonitrile was added 10 g of the ‘PS Amano’ lipase, and the mixture was vigorously stirred at 50° C. After 48 h, the ¹H NMR showed the conversion of the starting material was 50%. The reaction mixture was filtered through Celite and extracted with EA (250 ml*3). The combined organic phase were dried over Na₂SO₄ and concentrated. The crude product was purified by silica gel column using EA:Hex (20:1 to 4:1) to give (2S,3R)-1-(4-methoxyphenyl)-2-(2-methylprop-1-en-1-yl)-4-oxoazetidin-3-yl acetate (1(+)) as white solid with yield 48% (97% ee). The product was reacted with “PS Amano” (half as 5 g) again using same condition to make further purification with yield in two step as 43% (98.9% ee).

### (3R,4S)-3-hydroxy-1-(4-methoxyphenyl)-4-(2-methylprop-1-en-1-yl)azetidin-2-one (9-2)

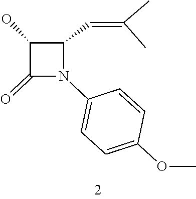

To a solution of potassium hydroxide (0.09 g, 1.6 mmol) in 1 ml water and 3 ml THF was added (2S,3R)-1-(4-methoxyphenyl)-2-(2-methylprop-1-en-1-yl)-4-oxoazetidin-3-yl acetate (9-1(+)) (0.289 g, 1 mmol) under 0° C. The mixture was then allowed to warm to room temperature and stirred for 3 h. After finished, the solvent was washed with brine, and extracted with EA (5 ml*2). The combined organic layer was dried with anhydrous Na₂SO₄, concentrated and purified by silica gel column using EA:Hex (4:1 to 1:1) to give (3R,4S)-3-hydroxy-1-(4-methoxyphenyl)-4-(2-methylprop-1-en-1-yl)azetidin-2-one (9-2) as white solid (yield 98%).

### (3R,4S)-1-(4-methoxyphenyl)-4-(2-methylprop-1-en-1-yl)-3-((triisopropylsilyl)oxy)azetidin-2-one (9-3)

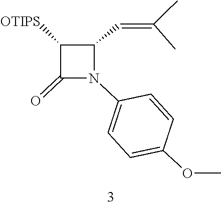

To a solution of (3R,4S)-3-hydroxy-1-(4-methoxyphenyl)-4-(2-methylprop-1-en-1-yl)azetidin-2-one (9-2) (2.47 g, 1 mmol) in DMF (10 ml) was added Et₃N (1.5 g, 1.5 mmol), 4-Dimethylaminopyridine (1.22 g, 1 mmol). Then TIPSCl (2.12 g, 1.1 mmol) was added over 10 min period. Stirred at room temperature for 3 h. After finished, EA (50 ml) was added, and the solvent was washed with water (10 ml*3) and brine. The organic layer was dried with anhydrous Na₂SO₄, concentrated and purified by silica gel column using EA:Hex (30:1 to 8:1) to give (3R,4S)-1-(4-methoxyphenyl)-4-(2-methylprop-1-en-1-yl)-3-((triisopropylsilyl)oxy)azetidin-2-one (9-3) as white solid (yield 97%).

### (3R,4S)-4-(2-methylprop-1-en-1-yl)-3-((triisopropylsilyl)oxy)azetidin-2-one (9-4)

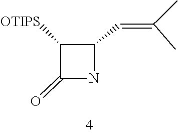

To a solution of (3R,4S)-1-(4-methoxyphenyl)-4-(2-methylprop-1-en-1-yl)-3-((triisopropylsilyl)oxy)azetidin-2-one (9-3) (2.19 g, 6 mmol) in acetonitrile (30 ml) was added dropwise ammonium cerium(IV) nitrate (10.15 g, 18.5 mmol) in H₂O over a 30 min period under 0° C. Then an additional H₂O (35 ml) was added over a 1 h period. Once finished, the mixture was diluted with 200 ml H₂O and extracted with EA (200 ml). The organic layer was treated with saturated NaHCO₃ (100 ml), saturated NaHSO₃ (100 ml), and again with saturated NaHCO₃ (100 ml). Washed with brine, and dried over anhydrous Na₂SO₄, concentrated under vacuum. The crude product was then purified by silica gel column using EA:Hex (10:1 to 3:1) to give (3R,4S)-4-(2-methylprop-1-en-1-yl)-3-((triisopropylsilyl)oxy)azetidin-2-one (9-4) as colorless solid in a yield of 63% yield.

### (3R,4S)-1-acryloyl-4-(2-methylprop-1-en-1-yl)-3-((triisopropylsilyl)oxy)azetidin-2-one (9-5)

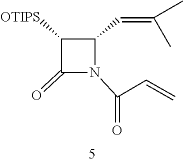

To a solution of (3R,4S)-4-(2-methylprop-1-en-1-yl)-3-((triisopropylsilyl)oxy)azetidin-2-one (9-4) (29.7 mg, 0.1 mmol) in dry CH₂Cl₂ (2 ml) was added Et₃N (0.07 ml, 0.5 mmol) and 4-Dimethylaminopyridine (12.2 mg, 0.1 mmol) under nitrogen atmosphere at 0° C. Then acryloyl chloride (27 mg, 0.3 mmol) was added. Stirred for 16 h. After finished, 10 ml EA was added and the solvent was washed with saturated NH₄Cl solvent, water, and then brine. The organic phase was dried over anhydrous Na₂SO₄, concentrated under vacuum. The crude product was then purified by silica gel column using EA:Hex (30:1 to 6:1) to give (3R,4S)-1-acryloyl-4-(2-methylprop-1-en-1-yl)-3-((triisopropylsilyl)oxy)azetidin-2-one (9-5) as colorless oil with 62% yield.

### pent-4-en-1-yl 4-methylbenzenesulfonate (6)

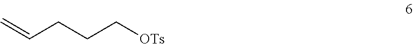 [SMILES: C=CCCCOS(=O)(=O)c1ccc(C)cc1]

To a solution of pent-4-en-1-ol (0.86 g, 10 mmol) in pyridine (5 ml) was added 4-Toluenesulfonyl chloride (4.19 g, 22 mmol) under 0° C. Stirred for 6 h. Once finished, 10 ml EA was added and the mixture was washed by 1 M HCl and 5% NaHCO₃ to remove pyridine. Then the organic layer was treated with brine, dried over anhydrous Na₂SO₄, concentrated under vacuum. The crude product was then purified by silica gel column using EA:Hex (20:1 to 4:1) to give pent-4-en-1-yl 4-methylbenzenesulfonate (9-6) as colorless oil with 88% yield.

### 5-azidopent-1-ene (9-7)

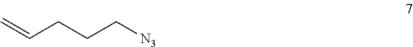

To a solution of pent-4-en-1-yl 4-methylbenzenesulfonate (9-6) (2.40 g, 10 mmol) in DMF (10 ml) was added sodium azide (1.05 g, 15 mmol) at room temperature. Stirred for 24 h. Once finished, the solution was quenched by H₂O (30 ml), then extracted with ether (10 ml*3). The combined organic layer was as treated with brine, dried over anhydrous Na₂SO₄, concentrated slowly to remove ether. The crude product was then used directly in next step.

### pent-4-en-1-amine (9-8)

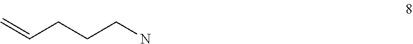

To a solution of 5-azidopent-1-ene (9-7) (1.11 g, 10 mmol) in ether (10 ml) was added PPh₃ (2.62 g, 10 mmol) under 0° C. Stirred for 1 h. Then H₂O (2 ml) was added, the solution was allowed to warm to room temperature and stirred for another 12 h. Once finished, the solution was quenched by ice water (10 ml), and the mixture was extracted with CH₂Cl₂ (10 ml*2). The combined organic layer was as treated with brine, dried over anhydrous Na₂SO₄, concentrated slowly to remove CH₂Cl₂. The crude product was then used directly in next step.

### tert-butyl pent-4-en-1-ylcarbamate (9-9)

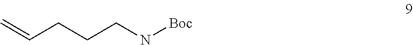

To a solution of pent-4-en-1-amine (9-8) (0.85 g, 10 mmol) in CH₂Cl₂ (10 ml) was added Et₃N (2.5 g, 25 mmol) and Boc-anhydride (3.27 g, 15 mmol) at room temperature. Stirred for 6 h. After finished, the solvent was washed with saturated NH₄Cl, water, and then brine. The organic phase was dried over anhydrous Na₂SO₄, concentrated under vacuum. The crude product was then purified by silica gel column using EA:Hex (16:1 to 4:1) to give tert-butyl pent-4-en-1-ylcarbamate (9-9) as colorless oil in 51% yield in total 3 steps.

### tert-butyl (tert-butoxycarbonyl)(pent-4-en-1-yl)carbamate (9-10)

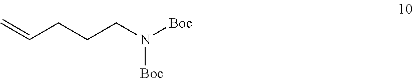 [SMILES: C=CCCCN(C(=O)OC(C)(C)C)C(=O)OC(C)(C)C]

To a solution of tert-butyl pent-4-en-1-ylcarbamate (9-9) (1.85 g, 10 mmol) in acetonitrile (15 ml) was added 4-Dimethylaminopyridine (0.24 g, 2 mmol). Then Boc-anhydride (2.4 g, 11 mmol) was added at 50° C. Stirred for 24 h. A further quantity of Boc-anhydride (1.2 g, 5.5 mmol) was added, and then reacted for an additional 24 h. After finished, the solvent was removed under vacuum and directly purified by silica gel column using EA:Hex (20:1 to 5:1) to give tert-butyl (tert-butoxycarbonyl)(pent-4-en-1-yl)carbamate (9-10) as colorless oil in 82% yield.

### tert-butyl (tert-butoxycarbonyl)((E)-6-((2S,3R)-2-(2-methylprop-1-en-1-yl)-4-oxo-3-((triisopropylsilyl)oxy)azetidin-1-yl)-6-oxohex-4-en-1-yl)carbamate (9-11)

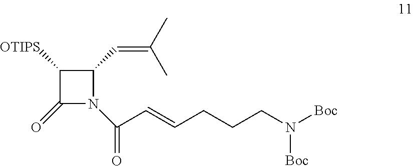

To a solution of (3R,4S)-1-acryloyl-4-(2-methylprop-1-en-1-yl)-3-((triisopropylsilyl)oxy)azetidin-2-one (9-5) (25 mg, 0.07 mmol) in CH₂Cl₂ (3 ml) was added tert-butyl (tert-butoxycarbonyl)(pent-4-en-1-yl)carbamate (9-10) (60.89 mg, 0.21 mmol) and Grubbs catalyst M720 (2 mg, 4%*0.07 mmol). The solvent was then allowed to warm to 50° C. to make CH₂Cl₂ refluxed. Stirred for 48 h. Once finished, the solvent was removed under vacuum and directly purified by silica gel column using EA:Hex (15:1 to 3:1) to give tert-butyl (tert-butoxycarbonyl)((E)-6-((2S,3R)-2-(2-methylprop-1-en-1-yl)-4-oxo-3-((triisopropylsilyl)oxy)azetidin-1-yl)-6-oxohex-4-en-1-yl)carbamate (9-11) as colorless oil in 91% yield.

### (2aR,4S,4aS,6R,9S,11S,12S,12aR,12bS)-12-(benzoyloxy)-4,9,11-trihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-12)

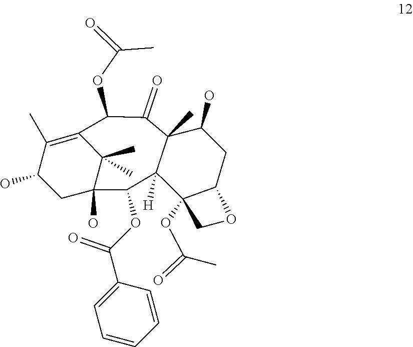

To a solution of 10-deacetylbaccatin (54.5 mg, 0.1 mmol) in THF (1 ml) was added cerium(III) chloride heptahydrate (1.86 mg, 0.005 mmol), then acetic anhydride (1 ml, 1 mmol) was added under 0° C. The reaction was allowed to slowly return to room temperature. Stirred for 4 h. After finished, the solution was quenched by ice water (10 ml), and the mixture was extracted with ethyl acetate (2 ml*2). The combined organic layer was as treated with brine, dried over anhydrous Na₂SO₄, concentrated under vacuum. The crude product was then purified by silica gel column using EA:Hex (30:1 to 6:1) to give (2aR,4S,4aS,6R,9S,11S,12S,12aR,12bS)-12-(benzoyloxy)-4,9,11-trihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-12) as white solid in 91% yield.

### (2aR,4S,4aS,6R,9S,11S,12S,12aR,12bS)-12-(benzoyloxy)-9,11-dihydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-13)

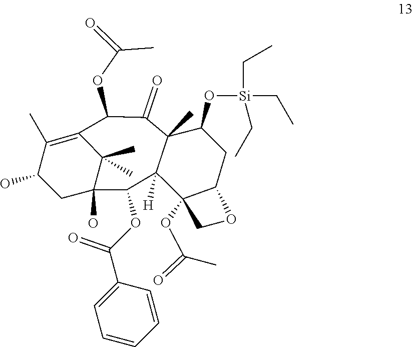

To a solution of (2aR,4S,4aS,6R,9S,11S,12S,12aR,12bS)-12-(benzoyloxy)-4,9,11-trihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-12) (0.293 g, 0.5 mmol) in dry CH₂Cl₂ (5 ml) was added 4-Dimethylaminopyridine (0.184 g, 1.5 mmol) under nitrogen atmosphere. Then chlorotriethylsilane (0.151 g, 1 mmol) was added dropwise. Stirred for 2 h at room temperature. Once finished, treated with water and 1 M HCl. Then the organic layer was washed with brine, dried over anhydrous Na₂SO₄, concentrated under vacuum. The crude product was then purified by silica gel column using EA:Hex (4:1 to 1:1) to give (2aR,4S,4aS,6R,9S,11S,12S,12aR,12bS)-12-(benzoyloxy)-9,11-dihydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-13) as white solid in 77% yield.

### (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-5-methyl-2-((triisopropylsilyl)oxy)hex-4-enoyl)oxy)-11-hydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-14)

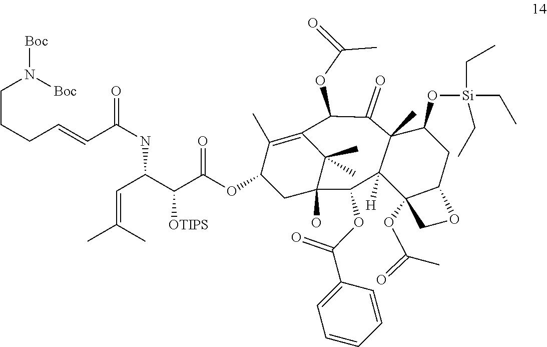

To a mixture of (2aR,4S,4aS,6R,9S,11S,12S,12aR,12bS)-12-(benzoyloxy)-9,11-dihydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-13) (50 mg, 0.07 mmol) and tert-butyl (tert-butoxycarbonyl)((E)-6-((2S,3R)-2-(2-methylprop-1-en-1-yl)-4-oxo-3-((triisopropylsilyl)oxy)azetidin-1-yl)-6-oxohex-4-en-1-yl)carbamate (9-11) (433 mg, 0.7 mmol) was added dry THF (1 ml). The solvent was then cooled to −78° C. 1.0 M LiHMDS in THF (0.7 ml, 7 mmol) was added dropwise to the system, stirred for 1 h. After finished, the solvent was directly quenched by saturated NH₄Cl (5 ml), and then washed with water, brine. The crude product was then purified by silica gel column using EA:Hex (4:1 to 1:1) to give (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-5-methyl-2-((triisopropylsilyl)oxy)hex-4-enoyl)oxy)-11-hydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-14) as white solid in 89% yield.

### (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-2-hydroxy-5-methylhex-4-enoyl)oxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-15)

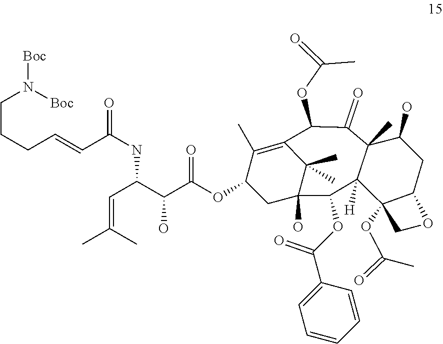

To a solution of (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-5-methyl-2-((triisopropylsilyl)oxy)hex-4-enoyl)oxy)-11-hydroxy-4a,8,13,13-tetramethyl-5-oxo-4-((triethylsilyl)oxy)-3,4,4a,5,6,9,10,11,12,12a-decahydro-1H-7,11-methanocyclodeca[3,4]benzo[1,2-b]oxete-6,12b(2aH)-diyl diacetate (9-14) (50 mg, 0.038 mmol) in 2 ml of a 1:1 (v/v, pyridine:acetonitrile) was added 0.5 ml HF/py. (7:3) under 0° C. Then the reaction was allowed to warm to room temperature and stirred for 24 h. After finished, the solvent was directly quenched by saturated NaHCO₃ (5 ml), and then extracted with ethyl acetate (10 ml*2). The combined organic phase was washed with saturated copper sulfate (5 ml) and brine, dried over anhydrous Na₂SO₄, concentrated under vacuum. The crude product was then purified by silica gel column using EA:Hex (4:1 to 1:1) to give (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-2-hydroxy-5-methylhex-4-enoyl)oxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-15) as white solid in 90% yield.

### (2aR,4S,4aS,6R,9S,11S,12S,12aR,12bS)-12-(benzoyloxy)-9-(((2R,3S)-3-((E)-6-(((((1R,8S,9s)-bicyclo[6.1.0]non-4-yn-9-yl)methoxy)carbonyl)amino)hex-2-enamido)-2-hydroxy-5-methylhex-4-enoyl)oxy)-4,11-dihydroxy-4a,8,13,13-tetramethyl-5-oxo-3,4,4a,5,6,9,10,11,12,12a-decahydro-1H-7,11-methanocyclodeca[3,4]benzo[1,2-b]oxete-6,12b(2aH)-diyl diacetate (9-16)

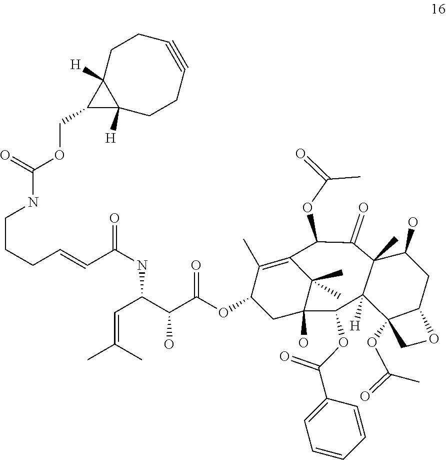

To a solution of (2aR,4S,4aS,6R,9S,11S,12S,12aR,12bS)-12-(benzoyloxy)-9-(((2R,3S)-3-((E)-6-(bis(tert-butoxycarbonyl)amino)hex-2-enamido)-2-hydroxy-5-methylhex-4-enoyl)oxy)-4,11-dihydroxy-4a,8,13,13-tetramethyl-5-oxo-3,4,4a,5,6,9,10,11,12,12a-decahydro-TH-7,11-methanocyclodeca[3,4]benzo[1,2-b]oxete-6,12b(2aH)-diyl diacetate (9-15) (5 mg, 0.005 mmol) in CH₂Cl₂ (0.2 ml) was added 0.2 ml TFA under nitrogen atmosphere. Then the solvent was stirred for 15 min. ESI showed that all compound 15 was di-deprotected Boc group. Then the CH₂Cl₂ and TFA was removed under vacuum. The residue was dissolved in 0.5 ml DMF, the Et₃N (0.1 ml) was added to make the pH=8˜ 9. ((1R,8S,9s)-bicyclo[6.1.0]non-4-yn-9-yl)methyl (4-nitrophenyl) carbonate (3 mg, 0.01 mmol) was added. Stirred for 24 h. Once finished, 2 ml ethyl acetate was added, and the mixture was washed by water (2 ml*3), brine. The organic phase was dried over anhydrous Na₂SO₄, concentrated under vacuum. The crude product was then purified by silica gel column using EA:Hex (3:1 to 1:1) to give (2aR,4S,4aS,6R,9S,11S,12S,12aR,12bS)-12-(benzoyloxy)-9-(((2R,3S)-3-((E)-6-(((((1R,8S,9s)-bicyclo[6.1.0]non-4-yn-9-yl)methoxy)carbonyl)amino)hex-2-enamido)-2-hydroxy-5-methylhex-4-enoyl)oxy)-4,11-dihydroxy-4a,8,13,13-tetramethyl-5-oxo-3,4,4a,5,6,9,10,11,12,12a-decahydro-TH-7,11-methanocyclodeca[3,4]benzo[1,2-b]oxete-6,12b(2aH)-diyl diacetate (9-16) as white solid.

### Conjugation

The above clickable drug-linker (compound 9-16) was conjugated to a Her2 antibody according the general procedure described above in Example 4. Full conversion of the modification was observed by native intact IgG analysis (ie. DAR=2).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

### Sequence Listing Part of the Description

SEQ ID NO. 1:
MIHTNLKKKFSCCVLVFLLFAVICVWKEKKKGSYYDSFKLQTKEFQVLKSLGKLAM
GSDSQSVSSSSTQDPHRGRQTLGSLRGLAKAKPEASFQVWNKDSSSKNLIPRLQKIWKNYLS
MNKYKVSYKGPGPGIKFSAEALRCHLRDHVNVSMVEVTDFPFNTSEWEGYLPKESIRTKAGP
WGRCAVVSSAGSLKSSQLGREIDDHDAVLRFNGAPTANFQQDVGTKTTIRLMNSQLVTTEK
RFLKDSLYNEGILIVWDPSVYHSDIPKWYQNPDYNFFNNYKTYRKLHPNQPFYILKPQMPWE
LWDILQEISPEEIQPNPPSSGMLGIIIMMTLCDQVDIYEFLPSKRKTDVCYYYQKFFDSACTMG
AYHPLLYEKNLVKHLNQGTDEDIYLLGKATLPGFRTIHC
SEQ ID NO. 2:
MRLREPLLSGSAAMPGASLQRACRLLVAVCALHLGVTLVYYLAGRDLSRLPQLVGV
STPLQGGSNSAAAIGQSSGELRTGGARPPPPLGASSQPRPGGDSSPVVDSGPGPASNLTSVPVP
HTTALSLPACPEESPLLVGPMLIEFNMPVDLELVAKQNPNVKMGGRYAPRDCVSPHKVAIIIP
FRNRQEHLKYWLYYLHPVLQRQQLDYGIYVINQAGDTIFNRAKLLNVGFQEALKDYDYTCF
VFSDVDLIPMNDHNAYRCFSQPRHISVAMDKFGFSLPYVQYFGGVSALSKQQFLTINGFPNN
YWGWGGEDDDIFNRLVFRGMSISRPNAVVGRCRMIRHSRDKKNEPNPQRFDRIAHTKETML
SDGLNSLTYQVLDVQRYPLYTQITVDIGTPS
SEQ ID NO. 3:
MDKHLLVKRTLGCVCAATLMGAALATHHDSLNTVKAEEKTVQVQKGLPSIDSLHY
LSENSKKEFKEELSKAGQESQKVKEILAKAQQADKQAQELAKMKIPEKIPMKPLHGPLYGGY
FRTWHDKTSDPTEKDKVNSMGELPKEVDLAFIFHDWTKDYSLFWKELATKHVPKLNKQGT
RVIRTIPWRFLAGGDNSGIAEDTSKYPNTPEGNKALAKAIVDEYVYKYNLDGLDVDVEHDSI
PKVDKKEDTAGVERSIQVFEEIGKLIGPKGVDKSRLFIMDSTYMADKNPLIERGAPYINLLLV
QVYGSQGEKGGWEPVSNRPEKTMEERWQGYSKYIRPEQYMIGFSFYEENAQEGNLWYDINS
RKDEDKANGINTDITGTRAERYARWQPKTGGVKGGIFSYAIDRDGVAHQPKKYAKQKEFKD
ATDNIFHSDYSVSKALKTVMLKDKSYDLIDEKDFPDKALREAVMAQVGTRKGDLERFNGTL
RLDNPAIQSLEGLNKFKKLAQLDLIGLSRITKLDRSVLPANMKPGKDTLETVLETYKKDNKEE
PATIPPVSLKVSGLTGLKELDLSGFDRETLAGLDAATLTSLEKVDISGNKLDLAPGTENRQIFD
TMLSTISNHVGSNEQTVKFDKQKPTGHYPDTYGKTSLRLPVANEKVDLQSQLLFGTVTNQGT
LINSEADYKAYQNHKIAGRSFVDSNYHYNNFKVSYENYTVKVTDSTLGTTTDKTLATDKEET
YKVDFFSPADKTKAVHTAKVIVGDEKTMMVNLAEGATVIGGSADPVNARKVFDGQLGSET
DNISLGWDSKQSIIFKLKEDGLIKHWRFFNDSARNPETTNKPIQEASLQIFNIKDYNLDNLLEN
PNKFDDEKYWITVDTYSAQGERATAFSNTLNNITSKYWRVVFDTKGDRYSSPVVPELQILGY
PLPNADTIMKTVTTAKELSQQKDKFSQKMLDELKIKEMALETSLNSKIFDVTAINANAGVLK
DCIEKRQLLKK
SEQ ID NO. 4:
HHHHHHWSHPQFEKGGGSGGGSGGSSAWSHPQFEKFQVLKSLGKLAMGSDS
QSVSSSSTQDPHRGRQTLGSLRGLAKAKPEASFQVWNKDSSSKNLIPRLQKIWKNYL
SMNKYKVSYKGPGPGIKFSAEALRCHLRDHVNVSMVEVTDFPFNTSEWEGYLPKESI
RTKAGPWGRCAVVSSAGSLKSSQLGREIDDHDAVLRFNGAPTANFQQDVGTKTTIR
LMNSQLVTTEKRFLKDSLYNEGILIVWDPSVYHSDIPKWYQNPDYNFFNNYKTYRK
LHPNQPFYILKPQMPWELWDILQEISPEEIQPNPPSSGMLGIIIMMTLCDQVDIYEFLPS
KRKTDVCYYYQKFFDSACTMGAYHPLLYEKNLVKHLNQGTDEDIYLLGKATLPGFR
TIHC
SEQ ID NO. 5:
HHHHHHHHHHRDLSRLPQLVGVSTPLQGGSNSAAAIGQSSGELRTGGARPPP
PLGASSQPRPGGDSSPVVDSGPGPASNLTSVPVPHTTALSLPACPEESPLLVGPMLIEF
NMPVDLELVAKQNPNVKMGGRYAPRDCVSPHKVAIIIPFRNRQEHLKYWLYYLHPV
LQRQQLDYGIYVINQAGDTIFNRAKLLNVGFQEALKDYDYTCFVFSDVDLIPMNDH
NAYRCFSQPRHISVAMDKFGFSLPYVQYFGGVSALSKQQFLTINGFPNNYWGWGGE
DDDIFNRLVFRGMSISRPNAVVGRCRMIRHSRDKKNEPNPQRFDRIAHTKETMLSDG
LNSLTYQVLDVQRYPLYTQITVDIGTPS
SEQ ID NO. 6:
EEKTVQVQKGLPSIDSLHYLSENSKKEFKEELSKAGQESQKVKEILAKAQQA
DKQAQELAKMKIPEKIPMKPLHGPLYGGYFRTWHDKTSDPTEKDKVNSMGELPKEV
DLAFIFHDWTKDYSLFWKELATKHVPKLNKQGTRVIRTIPWRFLAGGDNSGIAEDTS
KYPNTPEGNKALAKAIVDEYVYKYNLDGLDVDVEHDSIPKVDKKEDTAGVERSIQV
FEEIGKLIGPKGVDKSRLFIMDSTYMADKNPLIERGAPYINLLLVQVYGSQGEKGGW
EPVSNRPEKTMEERWQGYSKYIRPEQYMIGFSFYEENAQEGNLWYDINSRKDEDKA
NGINTDITGTRAERYARWQPKTGGVKGGIFSYAIDRDGVAHQPKKYAKQKEFKDAT
DNIFHSDYSVSKALKTVMLKDKSYDLIDEKDFPDKALREAVMAQVGTRKGDLERFN
GTLRLDNPAIQSLEGLNKFKKLAQLDLIGLSRITKLDRSVLPANMKPGKDTLETVLET
YKKDNKEEPATIPPVSLKVSGLTGLKELDLSGFDRETLAGLDAATLTSLEKVDISGNK
LDLAPGTENRQIFDTMLSTISNHVGSNEQTVKFDKQKPTGHYPDTYGKTSLRLPVAN
EKVDLQSQLLFGTVTNQGTLINSEADYKAYQNHKIAGRSFVDSNYHYNNFKVSYEN
YTVKVTDSTLGTTTDKTLATDKEETYKVDFFSPADKTKAVHTAKVIVGDEKTMMV
NLAEGATVIGGSADPVNARKVFDGQLGSETDNISLGWDSKQSIIFKLKEDGLIKHWR
FFNDSARNPETTNKPIQEASLQIFNIKDYNLDNLLENPNKFDDEKYWITVDTYSAQGE
RATAFSNTLNNITSKYWRVVFDTKGDRYSSPVVPELQILGYPLPNADTIMKTVTTAK
ELSQQKDKFSQKMLDELKIKEMALETSLNSKIFDVTAINANAGVLKDCIEKRQLLKK
HHHHHHHHHH
SEQ ID NO. 7:
FQVLKSLGKLAMGSDSQSVSSSSTQDPHRGRQTLGSLRGLAKAKPEASFQVW
NKDSSSKNLIPRLQKIWKNYLSMNKYKVSYKGPGPGIKFSAEALRCHLRDHVNVSM
VEVTDFPFNTSEWEGYLPKESIRTKAGPWGRCAVVSSAGSLKSSQLGREIDDHDAVL
RFNGAPTANFQQDVGTKTTIRLMNSQLVTTEKRFLKDSLYNEGILIVWDPSVYHSDIP
KWYQNPDYNFFNNYKTYRKLHPNQPFYILKPQMPWELWDILQEISPEEIQPNPPSSG
MLGIIIMMTLCDQVDIYEFLPSKRKTDVCYYYQKFFDSACTMGAYHPLLYEKNLVK
HLNQGTDEDIYLLGKATLPGFRTIHC
SEQ ID NO. 8:
RDLSRLPQLVGVSTPLQGGSNSAAAIGQSSGELRTGGARPPPPLGASSQPRPG
GDSSPVVDSGPGPASNLTSVPVPHTTALSLPACPEESPLLVGPMLIEFNMPVDLELVA
KQNPNVKMGGRYAPRDCVSPHKVAIIIPFRNRQEHLKYWLYYLHPVLQRQQLDYGI
YVINQAGDTIFNRAKLLNVGFQEALKDYDYTCFVFSDVDLIPMNDHNAYRCFSQPR
HISVAMDKFGFSLPYVQYFGGVSALSKQQFLTINGFPNNYWGWGGEDDDIFNRLVF
RGMSISRPNAVVGRCRMIRHSRDKKNEPNPQRFDRIAHTKETMLSDGLNSLTYQVLD
VQRYPLYTQITVDIGTPS
SEQ ID NO. 9:
EEKTVQVQKGLPSIDSLHYLSENSKKEFKEELSKAGQESQKVKEILAKAQQA
DKQAQELAKMKIPEKIPMKPLHGPLYGGYFRTWHDKTSDPTEKDKVNSMGELPKEV
DLAFIFHDWTKDYSLFWKELATKHVPKLNKQGTRVIRTIPWRFLAGGDNSGIAEDTS
KYPNTPEGNKALAKAIVDEYVYKYNLDGLDVDVEHDSIPKVDKKEDTAGVERSIQV
FEEIGKLIGPKGVDKSRLFIMDSTYMADKNPLIERGAPYINLLLVQVYGSQGEKGGW
EPVSNRPEKTMEERWQGYSKYIRPEQYMIGFSFYEENAQEGNLWYDINSRKDEDKA
NGINTDITGTRAERYARWQPKTGGVKGGIFSYAIDRDGVAHQPKKYAKQKEFKDAT
DNIFHSDYSVSKALKTVMLKDKSYDLIDEKDFPDKALREAVMAQVGTRKGDLERFN
GTLRLDNPAIQSLEGLNKFKKLAQLDLIGLSRITKLDRSVLPANMKPGKDTLETVLET
YKKDNKEEPATIPPVSLKVSGLTGLKELDLSGFDRETLAGLDAATLTSLEKVDISGNK
LDLAPGTENRQIFDTMLSTISNHVGSNEQTVKFDKQKPTGHYPDTYGKTSLRLPVAN
EKVDLQSQLLFGTVTNQGTLINSEADYKAYQNHKIAGRSFVDSNYHYNNFKVSYEN
YTVKVTDSTLGTTTDKTLATDKEETYKVDFFSPADKTKAVHTAKVIVGDEKTMMV
NLAEGATVIGGSADPVNARKVFDGQLGSETDNISLGWDSKQSIIFKLKEDGLIKHWR
FFNDSARNPETTNKPIQEASLQIFNIKDYNLDNLLENPNKFDDEKYWITVDTYSAQGE
RATAFSNTLNNITSKYWRVVFDTKGDRYSSPVVPELQILGYPLPNADTIMKTVTTAK
ELSQQKDKFSQKMLDELKIKEMALETSLNSKIFDVTAINANAGVLKDCIEKRQLLKK

What is claimed is:

1. A glycoconjugate having the formula:
[[Payload]ₓ-sialoside-Gal-GlcNAc]_{y}-CBA,
wherein CBA represents a cell binding agent;
GlcNAc represents N-acetylglucosamine, optionally substituted with fucose;
Gal represents galactose;
Sialoside represents a modified sialic acid;
Payload, which may be the same or different in each instance, represents a drug or diagnostic marker;
wherein the payload does not include a pyrrolobenzodiazepine;
x is an integer of 1-8; and
y is an integer of 1-8, preferably 1 or 2.

2. The glycoconjugate according to any preceding claim, having the formula: 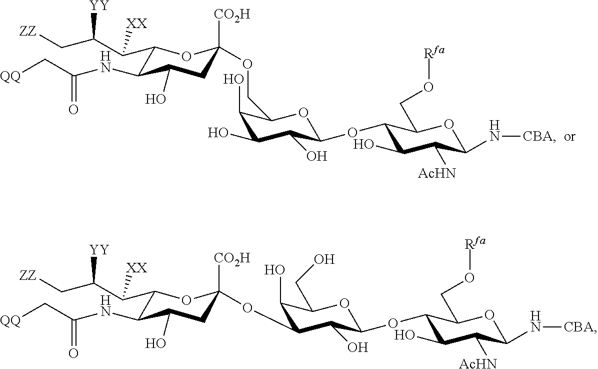
wherein
R^{fa} is a hydrogen or fucose moiety;
QQ is hydrogen, or at least one conjugated payload;
ZZ is hydroxyl, or at least one conjugated payload;
YY is hydroxyl, or at least one conjugated payload;
XX is hydroxyl, or at least one conjugated payload; and
wherein at least one of QQ, ZZ, YY, and XX is at least one conjugated payload.

3. The glycoconjugate according to any preceding claim, wherein R^{fa} is a fucose moiety.

4. The glycoconjugate according to any preceding claim, wherein R^{fa} is a fucose moiety having the structure:

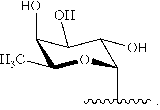

5. The glycoconjugate according to any preceding claim, wherein CBA is a protein.

6. The glycoconjugate according to any preceding claim, wherein CBA is an antibody.

7. The glycoconjugate according to any preceding claim, wherein CBA is a monoclonal antibody.

8. The glycoconjugate according to any preceding claim, wherein CBA is a protein, and the glycoconjugate has the structure:

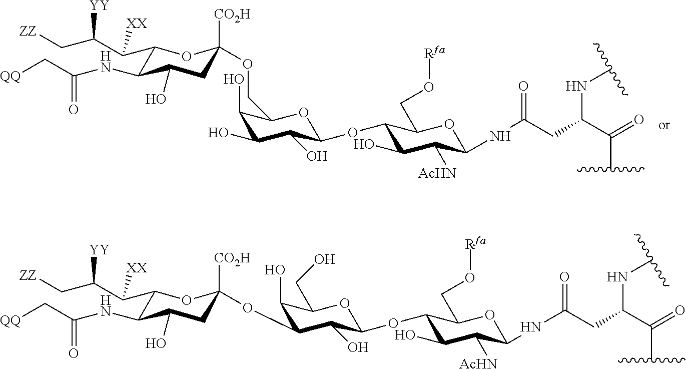

9. The glycoconjugate according to any preceding claim, wherein CBA is an antibody, and the GlcNAc is conjugated to the antibody via an asparagine side chain.

10. The glycoconjugate according to any preceding claim, wherein CBA is an antibody, and the GlcNAc is conjugated to at least one of the Asn297 residues in the Fc domain.

11. The glycoconjugate according to any preceding claim, wherein CBA is an antibody, and the GlcNAc is conjugated to both of the Asn297 residues in the Fc domain.

12. The glycoconjugate according to any preceding claim, wherein the payload comprises at least one cytotoxin, immunomodulator, antiviral agent, antibacterial agent, peptide or oligonucleotides.

13. The glycoconjugate according to any preceding claim, wherein the payload comprises a least one drug having a water solubility, at 23° C., of less than 20 mg/ml, less than 10 mg/ml, less than 5 mg/ml, less than 2.5 mg/ml, less than 1 mg/ml, less than 0.5 mg/ml, less than 0.1 mg/ml, less than 0.05 mg/ml, or less than 0.01 mg/ml.

14. The glycoconjugate according to any preceding claim, wherein the payload comprises a DNA damaging agent, a tubulin polymerization inhibitor, a topoisomerase inhibitor, a RNA splicing inhibitor, a RNE polymerase inhibitor, or a combination thereof.

15. The glycoconjugate according to any preceding claim, wherein the payload comprises a colchicine, a vinca alkaloid, an anthracycline, a camptothecin, doxorubicin, daunorubicin, a taxane, a dolastatin, an ansamitocin, a pyridinobenzodiazepine, an eribulin, a crytophycin, an ene-diyne antibiotic, a tubulysin, an exatecan, an irinotecan, a inhibitory peptide, an amanitin, a deBouganin, a duocarmycin, a maytansine, an auristatin, or a combination thereof.

16. The glycoconjugate according to any preceding claim, wherein at least one of QQ, XX, YY, and ZZ has the formula: 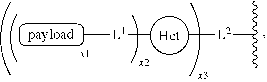
wherein Het represents a heterocyclic system, which may the same or different in each case;
L¹ is selected from null or sublinker, which may the same or different in each case;
L² is selected from null or sublinker, which may the same or different in each case;
x1 is selected from 1, 2, 3, 4, 5, 6, 7, or 8;
x2 is selected from 1, 2, 3, 4, 5, 6, 7, or 8; and
x3 is selected from 1, 2, 3, 4, 5, 6, 7, or 8.

17. The glycoconjugate according to any preceding claim, wherein:
a) XX, YY, and ZZ are each OH;
b) XX and YY are both OH, and QQ is H;
c) XX and YY are both OH.

18. The glycoconjugate according to any preceding claim, wherein QQ has the formula:

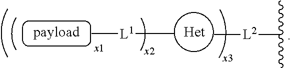

19. The glycoconjugate according to any preceding claim, wherein ZZ has the formula:

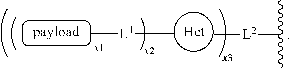

20. The glycoconjugate according to any preceding claim, wherein Het has the formula: 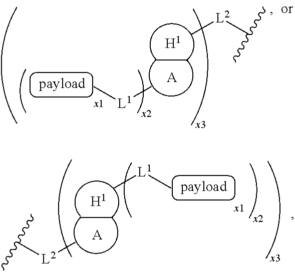
wherein H¹ represents a heterocyclic ring and A represents a carbocyclic or heterocyclic ring.

21. The glycoconjugate according to any preceding claim, wherein A is an 8-atom carbocyclic or heterocyclic ring.

22. The glycoconjugate according to any preceding claim, wherein H¹ is a heterocyclic ring formed from cycloaddition reaction between (a) either a 1,3 dipole or 1,2,4,5 tetrazine and (b) either a strained cycloalkyne or strained cycloalkene.

23. The glycoconjugate according to any preceding claim, wherein H¹ comprises a triazole, a 1,2 pyridazine, an oxazole, an isooxazole, an oxadiazole, and saturated and partially unsaturated analogs thereof.

24. The glycoconjugate according to any preceding claim, wherein A has the formula: 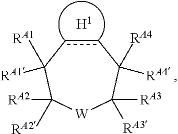
wherein R^{A1}, R^{A1′}, R^{A2}, R^{A2′}, R^{A3}, R^{A3′}, R^{A4}, and R^{A4′} are independently selected from null, H, F, Cl, Br, I, C₁₋₄alkyl, C₁₋₄alkoxy, aryl; and wherein any one of R^{A1}, R^{A1′}, R^{A2}, R^{A2′}, R^{A3}, R^{A3′}, R^{A4}, and R^{A4′} can be L¹ or L², wherein any two or more of R^{A1}, R^{A1′}, R^{A2}, R^{A2′}, R^{A3}, R^{A3′}, R^{A4}, and R^{A4′} can together form a ring;
W can be a group having the formula: 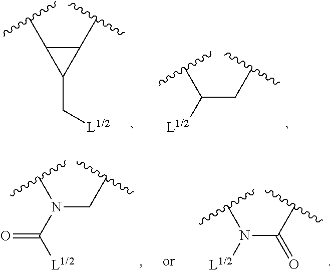
wherein L^{1/2} represents either H, L¹ or L²,
with the proviso that when one of W, R^{A1}, R^{A}, R^{A2}, R^{A2′}, R^{A3}, R^{A3′}, R^{A4}, and R^{A4′} includes L¹, none of W, R^{A1}, R^{A1′}, R^{A2}, R^{A2′}, R^{A3}, R^{A3′}, R^{A4}, and R^{A4′} includes L²; and L² is bonded to H¹; and
when one of W, R^{A1}, R^{A1′}, R^{A2}, R^{A2′}, R^{A3}, R^{A3′}, R^{A4}, and R^{A4′} includes L² none of W, R^{A1}, R^{A1′}, R^{A2}, R^{A2′}, R^{A3}, R^{A3′}, R^{A4}, and R^{A4′} includes L¹, and L¹ is bonded to H¹.

25. The glycoconjugate according to any preceding claim, wherein A ring has the formula:

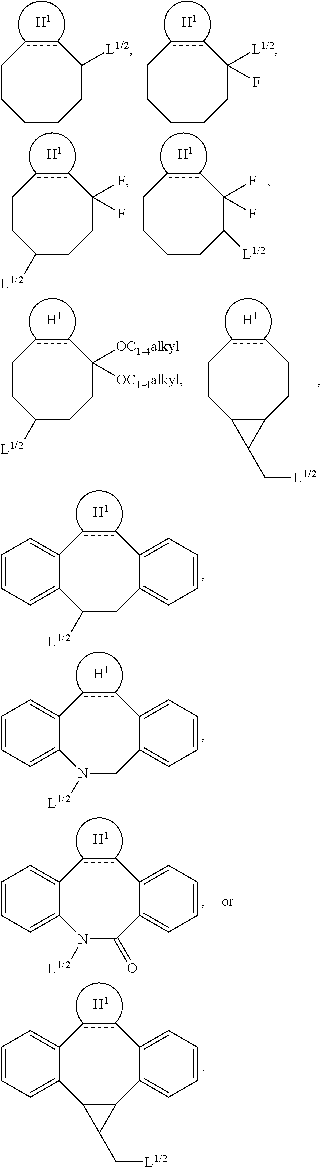

26. The glycoconjugate according to any preceding claim, wherein L² has the formula:
-L2¹-L2²-L2³-L2⁴-L2⁵-L2⁶-,
wherein:
L2¹ is bonded to the heterocyclic system and is selected from null, C₁₋₈alkylene, arylene, heteroaryl, heterocyclyl, poly(ethylene), poly(acetal); poly(glycerol), S, O, NR²¹, OC(═O), OC(═O)NR²¹, NR²¹C(═O), NR²¹C(═O)O, NR²¹C(═O), NR²¹C(═O)NR²¹, OC(═O)O, wherein R²¹ is in each case selected from H and C₁₋₄alkyl;
L2² is selected from null, C₁₋₈alkylene, arylene, heteroaryl, heterocyclyl, poly(ethylene), poly(acetal); poly(glycerol), S, O, NR²², OC(═O), OC(═O)NR²², NR²²C(═O), NR²²C(═O), NR²²C(═O)O, NR²²C(═O)NR²², OC(═O)O, wherein R²² is in each case selected from H and C₁₋₄alkyl;
L2³ is selected from null, C₁₋₈alkylene, arylene, heteroaryl, heterocyclyl, S, poly(ethylene), poly(acetal); poly(glycerol), O, NR²³, OC(═O), OC(═O)NR²³, NR²³C(═O), NR²³C(═O), NR²³C(═O)O, NR²³C(═O)NR²³, OC(═O)O, wherein R²³ is in each case selected from H and C₁₋₄alkyl;
L2⁴ is selected from null, C₁₋₈alkylene, arylene, heteroaryl, heterocyclyl, poly(ethylene), poly(acetal); poly(glycerol), S, O, NR²⁴, OC(═O), OC(═O)NR²⁴, NR²⁴C(═O), NR²⁴C(═O), NR²⁴C(═O)O, NR²⁴C(═O)NR²⁴, OC(═O)O, wherein R²⁴ is in each case selected from H and C₁₋₄alkyl;
L2⁵ is selected from null, C₁₋₈alkylene, arylene, heteroaryl, heterocyclyl, poly(ethylene), poly(acetal); poly(glycerol), S, O, NR²⁵, OC(═O), OC(═O)NR²¹, NR²⁵C(═O), NR²⁵C(═O), NR²⁵C(═O)O, NR²⁵C(═O)NR²¹, OC(═O)O, wherein R²¹ is in each case selected from H and C₁₋₄alkyl;
L2⁶ is bonded to the sialoside and is selected from null, C₁₋₈alkylene, arylene, heteroaryl, heterocyclyl, poly(ethylene), poly(acetal); poly(glycerol), S, O, NR²⁶, OC(═O), OC(═O)NR²⁶, NR²⁶C(═O), NR²⁶C(═O), NR²⁶C(═O)O, NR²⁶C(═O)NR²⁶, OC(═O)O, wherein R²⁶ is in each case selected from H and C₁₋₄alkyl.

27. The glycoconjugate according to any preceding claim, wherein
L2¹ is null, OC(O)NH, C₁₋₈alkylene, preferably C₁₋₃alkylene or arylene, for instance 1,4-phenylene;
L2² is null or C₁₋₈alkylene, preferably C₁₋₃alkylene;
L2³ is null, C(═O)NH, NHC(═O), NHC(═O)O, or OC(═O)NH;
L2⁴ is null or poly(ethylene),
L2⁵ is null or C₁₋₈alkylene, preferably C₁₋₃alkylene, and
L2⁶ is null or heterocyclyl or heteroaryl, for instance a triazole, a 1,2 pyridazine, an oxazole, an isooxazole, an oxadiazole, and saturated and partially unsaturated analogs thereof.

28. The glycoconjugate according to any preceding claim, wherein L¹ is null, or a group having the formula:
-L1¹-L1²-L1³-(L1⁴-L1⁵-L1⁶)ₓ-,
wherein:
x is selected from 1, 2, 3, 4, 5, 6, 7, or 8;
L1¹ is bonded to the heterocyclic system and is selected from null, C₁₋₈alkylene, arylene, heteroaryl, heterocyclyl, poly(ethylene), poly(acetal); poly(glycerol), S, O, NR¹¹, OC(═O), OC(═O)NR¹¹, NR¹¹C(═O), NR¹¹C(═O), NR¹¹C(═O)NR¹¹, OC(═O)O, wherein R¹¹ is in each case selected from H and C₁₋₄alkyl;
L1² is selected from null, C₁₋₈alkylene, arylene, heteroaryl, heterocyclyl, poly(ethylene), poly(acetal); poly(glycerol), S, O, NR¹², OC(═O), OC(═O)NR¹², NR¹²C(═O), NR¹²C(═O), NR¹²C(═O)NR¹², OC(═O)O, wherein R¹² is in each case selected from H and C₁₋₄alkyl;
L1³ is selected from null, C₁₋₈alkylene, arylene, heteroaryl, heterocyclyl, poly(ethylene), poly(acetal); poly(glycerol), S, O, NR¹³, OC(═O), OC(═O)NR¹³, NR¹³C(═O), NR¹³C(═O), NR¹³C(═O)NR¹³, OC(═O)O, wherein R¹³ is in each case selected from H and C₁₋₄alkyl;
L1⁴ is selected from null, C₁₋₈alkylene, arylene, heteroaryl, heterocyclyl, poly(ethylene), poly(acetal); poly(glycerol), S, O, NR¹⁴, OC(═O), OC(═O)NR¹⁴, NR¹⁴C(═O), NR¹⁴C(═O), NR¹⁴C(═O)NR¹⁴, OC(═O)O, wherein R¹⁴ is in each case selected from H and C₁₋₄alkyl;
L1⁵ is selected from null, C₁₋₈alkylene, arylene, heteroaryl, heterocyclyl, poly(ethylene), poly(acetal); poly(glycerol), S, O, NR¹⁵, OC(═O), OC(═O)NR¹⁵, NR¹⁵C(═O), NR¹⁵C(═O), NR¹⁵C(═O)NR¹⁵, OC(═O)O, wherein R¹⁵ is in each case selected from H and C₁₋₄alkyl;
L1⁶ is bonded to the payload and is selected from null, C₁₋₈alkylene, arylene, heteroaryl, heterocyclyl, poly(ethylene), poly(acetal); poly(glycerol), S, O, NR¹⁶, OC(═O), OC(═O)NR¹⁶, NR¹⁶C(═O), NR¹⁶C(═O), NR¹⁶C(═O)NR¹⁶, OC(═O)O, wherein R¹⁶ is in each case selected from H and C₁₋₄alkyl.

29. The glycoconjugate according to any preceding claim, wherein L¹ has the formula: 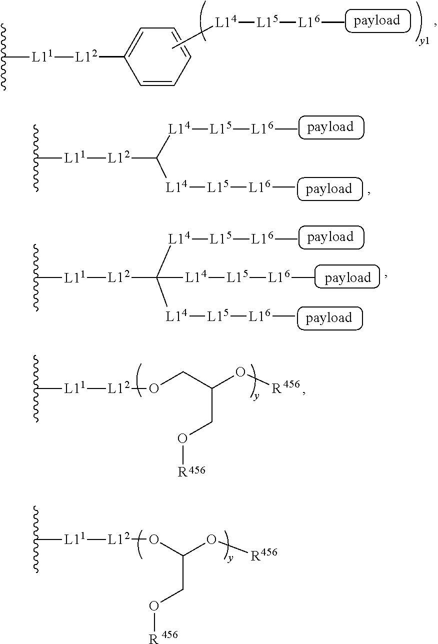
wherein y1 is selected from 1, 2, 3, 4, and 5; wherein y is from 1-1,000; and R⁴⁵⁶ is selected from hydrogen or a moiety of Formula (456):

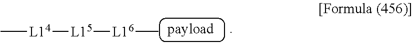

30. The glycoconjugate according to any preceding claim, wherein L¹ has the 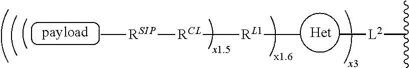
formula 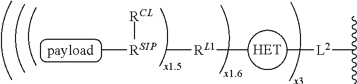
wherein R^{SIP} is one or more self-immolative spacers, R^{CL} is a cleavable group, and R^{L1}, when present, is an additional linker x1.5 is an integer selected from 1, 2, 3, 4, 5, 6, 7, and 8; and x1.6 is an integer selected from 1, 2, 3, 4, 5, 6, 7, and 8.

31. The glycoconjugate according to any preceding claim, wherein R^{SIP} has the formula: 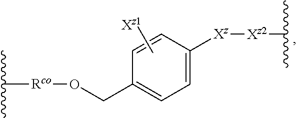
wherein X^{z} is O, NH, or NC₁₋₄alkyl;
R^{co} is bonded to a heteroatom in the payload, and is selected from C═O, SO₂, P(═O)OH, or a group having the formula: 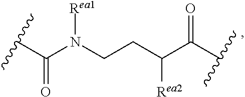
wherein R^{ea1} and R^{ea2} are independently selected from H and C₁₋₄alkyl; and
either:
a) X^{z1} is hydrogen and X^{z2} is R^{CL}, wherein R^{CL} is bonded to R^{L1}; or
b) X^{z1} is R^{L1} and X^{z2} is R^{CL}. 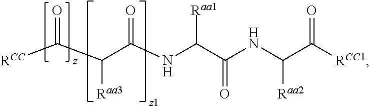
wherein z is 1 or 0, z1 is 1 or 0, R^{aa1}, R^{aa2}, and R^{aa3} are independently selected from H, C₁₋₆alkyl optionally substituted with phenyl, COOH, NH₂, COHNH₂, NHC(O)NH₂; and
either:
a) R^{CC} is H, peptidyl, C₁₋₆alkyl, cycloalkyl, aryl, heteroaryl, or heterocyclyl, and R^{CC1} is R^{SIP}, wherein said R^{SIP} is further bonded both to R^{L1} and the payload; or
b) R^{CC} is R^{L1} and R^{CC1} is R^{SIP}, wherein said R^{sP} is bonded to the payload.

32. The glycoconjugate according to any preceding claim, wherein R^{CL} has the formula:

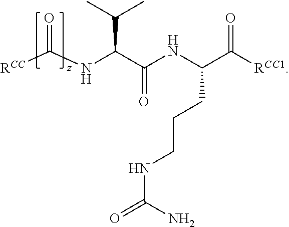

33. The glycoconjugate according to any preceding claim, wherein R^{CL} has the formula: 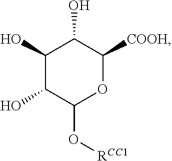
wherein R^{CC1} is R^{SIP}, wherein said R^{SIP} is further bonded both to R^{L1} and the payload.

34. The glycoconjugate according to any preceding claim, wherein R^{CL} has the formula: 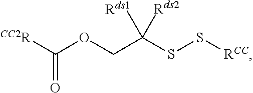
wherein R^{CC2} is R^{SIP}, or a heteroatom in the payload, and R^{CC} is L¹.

35. The glycoconjugate according to any preceding claim, wherein R^{CL} has the formula:

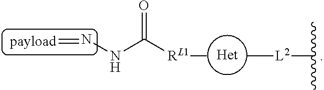

36. The glycoconjugate according to any one of claims 2 to 35, wherein at least one of QQ, XX, YY, and ZZ is at least one conjugated payload having the formula: 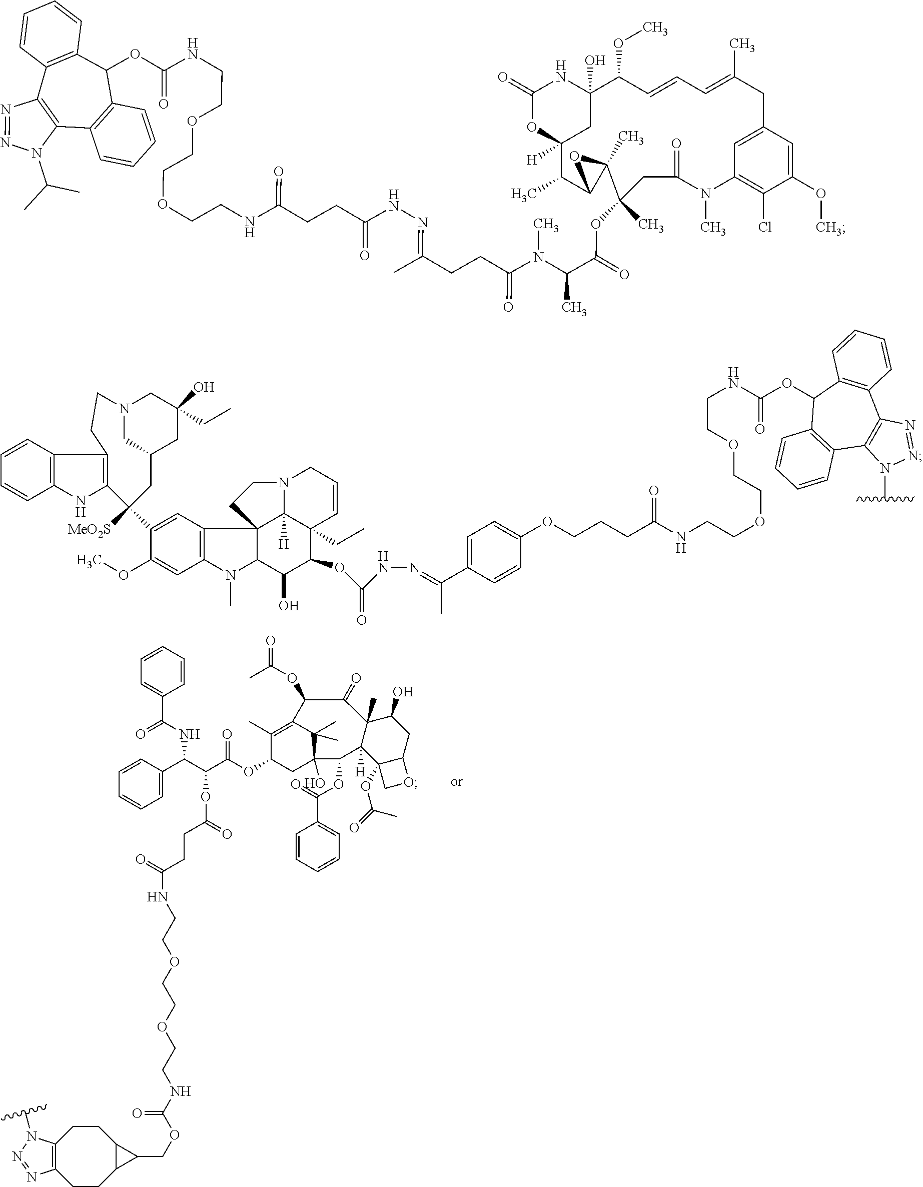 [chemical structure]


SEQUENCE LISTING (9 sequences)

SEQ ID NO: 1 - protein, 406 aa, Artificial Sequence
  synthetic construct
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 398 aa, Artificial Sequence
  synthetic construct
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 3 - protein, 995 aa, Artificial Sequence
  synthetic construct
MDKHLLVKRTLGCVCAATLMGAALATHHDSLNTVKAEEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 4 - protein, 397 aa, Artificial Sequence
  synthetic construct
HHHHHHWSHPQFEKGGGSGGGSGGSSAWSHPQFEKFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 5 - protein, 364 aa, Artificial Sequence
  synthetic construct
HHHHHHHHHHRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 6 - protein, 969 aa, Artificial Sequence
  synthetic construct
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKKHHHHHHHHHH

SEQ ID NO: 7 - protein, 362 aa, Artificial Sequence
  synthetic construct
FQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 8 - protein, 354 aa, Artificial Sequence
  synthetic construct
RDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 9 - protein, 959 aa, Artificial Sequence
  synthetic construct
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK